(19) United States
(12) Patent Application Publication
(10) Pub. No.: US 2023/0287480 A1
(43) Pub. Date: Sep. 14, 2023

(54) ANALYTE DETECTION SYSTEMS AND METHODS OF USE

(71) Applicant: Nautilus Subsidiary, Inc., Seattle, WA (US)

(72) Inventors: David STERN, Mountain View, CA (US); Glenn CHAN, Fremont, CA (US); Ezra VAN GELDER, Palo Alto, CA (US)

(21) Appl. No.: 18/180,733

(22) Filed: Mar. 8, 2023

Related U.S. Application Data
(60) Provisional application No. 63/269,161, filed on Mar. 10, 2022.
(60) Provisional application No. 63/377,604, filed on Sep. 29, 2022.

(51) Int. Cl. C12Q 1/6837; C12Q 1/6825; C12Q 1/6876

(52) U.S. Cl. CPC C12Q 1/6837; C12Q 1/6825; C12Q 1/6876; C12Q 2600/158

(57) ABSTRACT

Systems for optically detecting single-analytes, such as cells, nucleic acids, and polypeptides, are described. The described optical detection systems are suitable for multiplexed detection of single-analytes, including single-analytes provided in an array-based format. Methods for identifying single-analyte properties and interactions utilizing optical detection systems are provided.

### CROSS-REFERENCE TO RELATED APPLICATIONS

This application claims priority to U.S. Provisional Application No. 63/269,161, filed on Mar. 10, 2022, and U.S. Provisional Application No. 63/377,604, filed on Sep. 29, 2022, each of which is incorporated herein by reference in its entirety.

### BACKGROUND

Detection of analytes, such as cells, nucleic acids, and polypeptides, at single molecule resolution provides increased information on the properties and interactions of analytes that is not readily acquired from detection of such molecules in bulk. While bulk detection methods can provide an ensemble average measurement of a molecular property, only single-analyte detection can identify and/or quantify the specific properties or interactions of molecules within an ensemble of molecules. Identification of single-analyte properties or interactions may be especially significant in certain diagnostic or clinical systems where system dysfunction can initiate or propagate from a minute subfraction of analytes within the system. For example, protein aggregation processes may arise due to the presence of a small number of damaged or misfolded proteins. Bulk analyte assay methods can fail to observe the presence of such a small number of analytes within the ensemble observations due to their limited contribution to the overall measurement. In contrast, single-analyte measurements can observe properties or interactions for each analyte in an ensemble of analytes, thereby producing relevant information that may be lost in the bulk measurement.

A single-analyte system can be configured to detect single-analytes with sufficient resolution that the properties or interactions of each observed analyte are distinguishable from those of every other observed analyte within the system. Aspects of resolution for a single-analyte in a single-analyte system can include the single-analyte producing a detectable signal above a background signal for the system, and the single-analyte being sufficiently separated from adjacent single-analytes to produce a distinct signal. In single-analyte system with optically-based detection, fluorescent or luminescent moieties are often used to provide information on the presence, location, properties, and/or interactions of single-analytes.

### SUMMARY

In an aspect, provided herein is a method of detecting one or more probe binding interactions on a single-analyte array, the method comprising: a) obtaining the single-analyte array comprising a first address that comprises a first probe complex and a second address that comprises a second probe complex, wherein the first probe complex comprises a first affinity reagent bound to a first single-analyte, wherein the first affinity reagent is configured to produce a first detectable signal, wherein the second probe complex comprises a second affinity reagent bound to a second single-analyte, wherein the second affinity reagent is configured to produce a second detectable signal, wherein the first detectable signal is optically distinguishable from the second detectable signal, and wherein the first address is spatially resolvable from the second address at single-analyte resolution; b) contacting the first address with a first excitation source, thereby producing the first detectable signal, wherein the first excitation source does not produce a detectable signal at the second address while contacting the first address; c) contacting the second address with a second excitation source, thereby producing the second detectable signal, wherein the second excitation source does not produce a detectable signal at the first address while contacting the first address; and d) detecting the first signal and the second signal on a sensor, wherein the sensor comprises a first channel that is configured to detect the first signal and a second channel that is configured to detect the second signal.

In another aspect, provided herein is a method of detecting one or more probe binding interactions on a single-analyte array, the method comprising: a) obtaining the single-analyte array comprising a first address and a second address, wherein the first address comprises a first signal source that emits a first signal and the second address comprises a second signal source that emits a second signal, wherein the first address is spatially resolvable from the second address; and b) detecting the first signal and the second signal on an optical system comprising a sensor, wherein the sensor comprises a first channel that detects the first signal and a second channel that detects the second signal, wherein the first channel is spatially separated and coplanar with the second channel.

In another aspect, provided herein is a method of detecting one or more probe binding interactions on a single-analyte array, the method comprising: a) translating the relative positions of the single-analyte array and an optical system in time-delay integration mode, wherein the single-analyte array comprises a first address and a second address, wherein the first address comprises a first optical signal source that emits a first optical signal and the second address comprises a second optical signal source that emits a second optical signal, wherein the first address is physically separated from the second address by a distance that is resolvable at single-analyte resolution; and b) detecting the first signal and the second signal on a sensor, wherein the first optical signal is detected on a first channel of the sensor and the second optical signal is detected on a second channel of the sensor, and wherein focus of the first optical signal and the second optical signal on the sensor are not separately adjusted over the duration of a time-delay integration scan of the single-analyte array.

In another aspect, provided herein is a multiplex optical system, comprising: a) two or more light sources, wherein a first light source is configured to transmit light of a first wavelength, and wherein a second light source is configured to transmit light of a second wavelength, wherein the first wavelength differs from the second wavelength; b) a stage that is configured to accommodate a fluidic cartridge; c) an objective lens; and d) an optical sensor, wherein the optical sensor comprises a first channel and a second channel, wherein the first channel and the second channel are spatially separated on the sensor, and wherein the first channel is configured to detect the light of the first wavelength, and wherein the second channel is configured to detect the light of the second wavelength.

In another aspect, provided herein is a method, comprising: a) providing a solid support comprising an address, wherein the address is resolvable at single-analyte resolution, wherein the address comprises an analyte, wherein the analyte is coupled to the address by a linking moiety, wherein the linking moiety comprises a first optically detectable label, wherein a probe is bound to the analyte, wherein the probe comprises a second optically detectable label, wherein the first optically detectable label produces a first optical signal of a first wavelength, wherein the second optically detectable label produces a second optical signal of a second wavelength, and wherein the first wavelength differs from the second wavelength, b) detecting on a first channel of a sensor a presence of the first detectable signal at the address, and detecting on a second channel of the sensor a presence of the second detectable signal, c) removing the probe from the analyte, and d) after removing the probe from the analyte, detecting on the first channel of the sensor the presence of the first detectable signal, and detecting on the second channel an absence of the second detectable signal.

In some embodiments, detecting on the sensor occurs in time delay and integration (TDI) mode.

In some embodiments, the first channel comprises a first array of light-sensing elements, and the second channel comprises second array of light-sensing elements. In some embodiments, the first channel and the second channel are coplanar and spatially separated on a solid support.

In some embodiments, the linking moiety comprises a nucleic acid. In some embodiments, the nucleic acid comprises a nucleic acid nanoparticle.

In some embodiments, the probe further comprises an affinity agent. In some embodiments, the probe further comprises a plurality of affinity agents. In some embodiments, the plurality of affinity agents is coupled to the second detectable label by a retaining moiety. In some embodiments, the retaining moiety comprises a nucleic acid nanoparticle.

In some embodiments, the probe is non-covalently bound to the analyte. In some embodiments, removing the probe comprises dissociating the probe from the analyte.

In some embodiments, the probe is covalently bound to the analyte. In some embodiments, removing the probe comprises chemically or enzymatically separating the probe from the analyte. In some embodiments, chemically separating the probe from the analyte comprises performing an Edman-type degradation reaction.

In some embodiments, the method further comprises: e) contacting a second probe to the array containing the analyte, wherein the second probe comprises the second optically detectable label. In some embodiments, the method further comprises: f) detecting on the second channel of the sensor a presence or an absence of the second detectable signal at the address. In some embodiments, a temperature change of at least 1 degree Celsius occurs between step b) and step f). In some embodiments, the method further comprises, before step b), adjusting a focus of an optical detection system. In some embodiments, the focus of the optical detection system is not adjusted between step b) and step f).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B illustrate schematics of optical detection systems that are configured for multiplex detection of single-analytes, in accordance with some embodiments.

FIGS. 2A, 2B, 2C, 2D, 2E, and 2F depict multiplexed scanning of a single-analyte array by an optical detection system, in accordance with some embodiments.

FIGS. 3A, 3B, 3C, and 3D show various views of a multi-channel sensor, in accordance with some embodiments.

FIGS. 4A and 4B display detailed schematics of an illumination pathway and emission pathway, respectively, for a multiplexed optical detection system, in accordance with some embodiments.

FIGS. 5A and 5B illustrate optical detection systems that incorporate multiple single-channel sensors or one multi-channel sensor, respectively, in accordance with some embodiments.

FIG. 6 depicts an optical detection system that contains one or more heat sources, in accordance with some embodiments.

FIG. 7 shows a multiplexed optical detection system containing a multi-channel sensor, in accordance with some embodiments.

FIG. 8 displays various embodiments of single-analyte signal sources, in accordance with some embodiments.

FIG. 9 illustrates detection of a probe complex comprising a single-analyte, in accordance with some embodiments.

FIGS. 10A, 10B, 10C, and 10D depict various time steps of a multi-cycle time delay-integration scan of a single-analyte array using an optical detection system, in accordance with some embodiments.

### DETAILED DESCRIPTION

Several approaches exist to increase the throughput and efficiency of optical detection in single-analyte systems. Achieving single-analyte resolution in an optical system necessitates collecting a sufficient amount of light from a region containing a single-analyte to distinguish the single-analyte from the background while also possessing sufficient optical distinction to distinguish a location of a first single-analyte and a separate location of a second single-analyte. Accordingly, as the quantity and/or density of single-analytes to be detected by a single-analyte optical detection system is increased, design choices must be made to maintain a sufficient level of single-analyte resolution. For example, it may be desirable to achieve a high degree of fluorescent labeling of polypeptides on an array of single polypeptides, but as the array density is increased (i.e., a decrease in average spacing between adjacent single polypeptides), a high degree of labeling may promote optical cross-talk between adjacent polypeptides, thereby decreasing the optical distinction between the adjacent polypeptides. In such a system, maintaining single-analyte resolution as analyte array density is increased can be achieved by chemical approaches (e.g., decreased degree of labeling), engineering methods (e.g., altered numerical aperture, altered scan time or rate, etc.), or combinations of both.

Single-analyte optical detection systems that utilize fluorescence to generate optical signal are often further limited by the progressive nature of photodegradation or photodamage to the system. Photodegradation or photodamage to single-analytes may arise by several mechanisms, including photobleaching of fluorescent moieties associated with a single-analyte, and photolytic disruption of the structure of the single-analyte itself. For example, in single-molecule peptide fluorosequencing assays, photodamage to the peptide may lead to missed sequence reads or truncation of the peptide, with the probability of photodamage increasing for each successive cycle of sequence reads. Likewise, in affinity agent-based proteomic methods (e.g., polypeptide identification, polypeptide sequencing), in which binding interactions of fluorescently-labeled affinity agents with polypeptides are measured, successively-increasing photodamage may promote false positive or false negative binding interaction detections as polypeptide structure (e.g., primary, secondary, tertiary, or quaternary structure) becomes altered. Accordingly, it may be advantageous to provide optical detection systems that seek to provide single-analyte resolution of detection while minimizing a probability of photodegradation for examined single-analytes.

One technique for optical detection of single-analytes involves the use of time-delay integration (TDI) imaging. TDI imaging typically involves a relative translation between a fluorescent system (e.g., an array of single-analytes) and a detection sensor, thereby providing light from a fluorescing region to a larger number of pixels on the detection sensor at a typically lower level of light intensity for any given pixel. The translation rate of a TDI imaging system can be optimized to increase the throughput of detected single-analytes while decreasing the length of irradiation by a fluorescence excitation source, thereby decreasing the probability of photodegradation.

A technique for optical detection of single-analytes involves multiplexing of detection. Multiplexing of optical detection can pertain to the simultaneous or near-simultaneous detection of two or more differentiable optical signals. For example, a single-molecule nucleic acid sequencing system may utilize four different fluorescent labels with each fluorescent label fixed to correspond to one of the four DNA nucleotides. Multiplexing may be advantageous for increasing assay throughput and decreasing photodegradation by combining assay steps or cycles and reducing the total incidences of irradiation.

Multiplexing via the detection of multiple, distinct optical signals can require an optical system that can detect and distinguish each unique optical signal. For example, the use of four types of fluorophores, in which each fluorophore has a unique, characteristic emission wavelength, can require an optical sensing system that can differentiate light by wavelength. Often, the emission light pathway of multiplexed optical detection systems must contain additional optical components that parse emitted light by wavelength (e.g., beam splitters, dichroic mirrors, etc.) and direct each parsed fraction of light to a dedicated detector for that particular wavelength. Accordingly, a multiplexed optical detection system can contain multiple light pathways, with each light pathway necessitating at least occasional focusing independently of the focusing of other light pathways. A need for independent focal adjustment in a multiplexed optical system may be increased when the optical system has a non-isothermal operating environment, whether due to internal thermal changes caused by system heat sources (e.g., microprocessors, pumps, fans, etc.) or external thermal changes (e.g., diurnal temperature changes in a laboratory environment, etc.). Temperature-related effects (e.g., thermal expansion or contraction, etc.) on optical component materials and mechanical structures that accommodate them can produce non-uniform deviations in focal alignment for a multiplexed optical detection system with multiple light pathways. Accordingly, it is advantageous to simplify the layout of a multiplexed optical detection system to decrease the impact of non-isothermal operation.

Provided herein are systems and methods for optically detecting single-analytes and/or single-analyte interactions. The provided systems and methods may be especially useful for detection of fluorescence or luminescence by microscopy (e.g., confocal fluorescent microscopy). Moreover, the provided systems and methods may enhance an ability to identify and/or quantitate particular single-analyte interactions (e.g., binding interactions) at single-analyte resolution. In some embodiments, a provided optical detection system incorporates a single sensor containing multiple, spatially-separated detection channels, thereby permitting multiplexed optical detection through a single emission pathway. Further, in some embodiments, a provided optical system incorporates a spatially-separated, multi-wavelength illumination pathway that is configured to induce fluorescence in a pattern that is aligned with the multiple detection channels of a single sensor. An optical detection system, as set forth herein, may be particularly advantageous for the optical detection of single-analytes and/or single-analyte interactions, including high-throughput analysis of single-analytes in an array-based format.

### Definitions

As used herein, the term “single-analyte” refers to a chemical entity that is individually manipulated or distinguished from other chemical entities. The analyte can be, for example, a polypeptide (i.e. single-polypeptide), ligand-binding polypeptide (i.e. ligand-binding single-polypeptide), receptor (i.e. single-receptor), ligand (i.e. single-ligand), probe (i.e. single-probe) or other analyte set forth herein. A single-analyte, in some embodiments, possesses a distinguishing property such as volume, surface area, diameter, electrical charge, electrical field, magnetic field, electronic structure, electromagnetic absorbance, electromagnetic transmittance, electromagnetic emission, radioactivity, atomic structure, molecular structure, crystalline structure, or a combination thereof. The distinguishing property of a single-analyte, in some embodiments, is a property of the single-analyte that is detectable by a detection method that possesses sufficient spatial resolution to distinguish the individual single-analyte from any adjacent single-analytes. An analyte, in some embodiments, comprises a single molecule, a single complex of molecules, a single particle, or a single chemical entity comprising multiple conjugated molecules or particles. A single-analyte, in some embodiments, is distinguished based on spatial or temporal separation from other analytes, for example, in a system or method set forth herein. Moreover, reference herein to a ‘single-analyte’ in the context of a composition, system or method does not necessarily exclude application of the composition, system or method to multiple single-analytes that are manipulated or distinguished individually, unless indicated contextually or explicitly to the contrary.

As used herein, the term “single-analyte resolution,” when used in reference to a single-analyte array, refers to a detection of a single-analyte under the conditions that: 1) the single-analyte is detected by a signal with a magnitude that exceeds the magnitude of background signals for the detection system, and 2) the single-analyte is detected by a signal at a location that is spatially separated from the location of a signal corresponding to a different single-analyte (i.e., a spatial minimum of signal magnitude exists between a first single-analyte and a second single-analyte for the two single-analytes to be spatially resolved). In some cases, a signal corresponding to a first single-analyte, in some embodiments, is considered spatially resolved from a signal corresponding to a second single-analyte if a signal minimum occurs between the locations of the two single-analytes with a magnitude that is substantially less than an average or peak signal maximum of one or both signal maxima corresponding to the first and second single analytes. For example, a signal minimum between two signal maxima corresponding respectively to a first single analyte and a second single analyte, in some embodiments, has a magnitude that is no more than about 95%, 90%, 80%, 70%, 60%, 50%, 40%, 30%, 20%, 10%, 5%, 1%, or less than 1% of an average or peak signal maximum of the two signal maxima. In some cases, signals corresponding to two or more analytes, in some embodiments, is considered spatially resolved if a spatial resolution criterion is achieved, such as the Rayleigh Criterion. A signal magnitude (peak or average) corresponding to a single-analyte, in some embodiments, has a signal-to-noise ratio relative to an average background signal of at least about 1.1:1, 1.5:1, 2:1, 2.5:1, 3:1, 4:1, 5:1, 10:1, 20:1, 50:1, 100:1 or more than 100:1.

As used herein, the term “channel,” when used in reference to an optical sensor, refers to a portion of a sensor that is configured to detect a signal having particular characteristic(s) or lacking particular characteristic(s). A channel, in some embodiments, is configured to detect photons with a characteristic wavelength, for example, at the exclusion of photon at other wavelengths. A channel, in some embodiments, is configured to detect photons within a particular range of wavelengths, for example, at the exclusion of photon outside of the particular range of wavelengths. A channel, in some embodiments, detects photons from within a region of the electromagnetic spectrum (e.g., far infrared, near infrared, visible, near-ultraviolet, or far ultraviolet) or subregions thereof (e.g., red wavelengths, orange wavelengths, yellow wavelengths, etc.), for example, at the exclusion of photons from outside the region of subregion. Alternatively, a channel, in some embodiments, detects photons from outside those regions or subregions of the electromagnetic spectrum. A channel, in some embodiments, comprises an array of light-sensing elements (e.g., CCD, CMOS), such as a pixel array. Each light-sensing element of an array of light-sensing elements of a channel, in some embodiments, is configured to detect a signal with the same specific characteristic. An array of light-sensing elements of a channel, in some embodiments, comprises a mixture of light-sensing elements with varying detection characteristics that combine to provide a range of detection characteristics to a channel. For example, a channel, in some embodiments, comprises a mixture of red-sensing pixels (absorbing light with wavelengths between 620 nanometers (nm) and 750 nm) and orange-sensing pixels (absorbing light with wavelengths between 590 nm and 620 nm) to form a channel that detects light between 590 nm and 750 nm. A channel, in some embodiments, comprises an array of light-sensing elements that is spatially separated from an array of light-sensing elements belonging to a separate channel. An array of light-sensing elements, in some embodiments, comprises a mixture of different types of pixels, in which all pixels with the same detection properties comprise a channel. For example, a pixel array, in some embodiments, comprises a patterned array of 3 types of light-sensing elements (e.g., red, yellow, blue, red, yellow, blue, etc.), in which a red-sensing channel comprises each of the red-sensing pixels, a blue-sensing channel comprises each of the blue-sensing pixels, and a yellow-sensing channel comprises each of the yellow-sensing pixels.

As used herein, the term “signal source” refers to a physical or chemical entity that is configured to produce an optically-detectable signal, such as a fluorescent or luminescent photon emission. A signal source, in some embodiments, refers to a light-producing moiety (e.g., a fluorophore, a luminophore, a fluorescent protein, a fluorescent nanoparticle, etc.) that is configured to produce an optically-detectable signal. Optionally, a light-producing moiety, in some embodiments, is covalently or non-covalently coupled to a single-analyte. Optionally, a light-producing moiety, in some embodiments, is covalently or non-covalently coupled to a member of an affinity binding pair (e.g., an antibody, antibody fragment, or aptamer with a binding specificity for a single-analyte) or a member of receptor-ligand binding pair (e.g., streptavidin-biotin, SpyCatcher-SpyTag, SnoopCatcher-SnoopTag, SdyCatcher-SdyTag, etc.). A signal source, in some embodiments, comprises a probe complex, in which the probe complex comprises a single-analyte and an affinity agent that is bound to the single-analyte, in which the affinity agent comprises a light-producing moiety.

As used herein, the term “excitant,” when used in reference to a signal source, refers to a chemical or physical stimulus that causes or induces the emission of a signal from the signal source. An excitant comprising a chemical or physical stimulus, in some embodiments, includes a discrete chemical or physical stimulus or a stimulating field. Exemplary discrete excitants, in some embodiments, include photons (e.g., a photon with an excitation wavelength that is configured to cause fluorescent of a fluorophore), chemical reactants (e.g., a chemical reactant that causes formation of a fluorescent moiety), and chemical substrates (e.g., a substrate for horseradish peroxidase that becomes fluorescent upon enzymatic conversion). Exemplary field excitants, in some embodiments, include beams of fields of photons and thermal fields (e.g., by conductive, convective, or radiative heat transfer).

As used herein, the terms “same” or “identical,” when used in reference to a chemical or physical entity or a property thereof, refers to two of the chemical or physical entities or respective properties thereof being indistinguishable. For example, two polypeptides, in some embodiments, are considered the same if they both possess identical amino acid sequences. In another example, two fluorophores, in some embodiments, are considered the same if they both possess identical chemical structures. As used herein, the term “differing” or “different,” when used in reference to a chemical or physical entity or a property thereof, refers to two of the chemical or physical entities or respective properties thereof being distinguishable. For example, two polypeptides of the same protein, in some embodiments, are different if they have differing proteoforms (e.g., differing sets of post-translational modifications). In another example, two fluorophores, in some embodiments, are different if each fluorophore has an excitation or emission wavelength that detectably differs from the other fluorophore.

As used herein, the term “comprising” is intended herein to be open-ended, including not only the recited elements, but further encompassing any additional elements.

As used herein, the term “each,” when used in reference to a collection of items, is intended to identify an individual item in the collection but does not necessarily refer to every item in the collection. Exceptions can occur if explicit disclosure or context clearly dictates otherwise.

### Optical Detection Systems

Provided herein are optical detection systems that are configured for multiplexed detection of single-analytes and/or single-analyte interactions. The optical detection systems are readily adaptable to various types of microscopy (e.g., fluorescence microscopy, confocal microscopy, etc.), various illumination methods (e.g., brightfield illumination, darkfield illumination, etc.), and various scanning methods (e.g., point-scanning, rastering, time-delay integration (TDI), etc.). In a particularly favorable configuration, an optical detection system, as set forth herein, utilizes a single, multi-channel sensor to detect multiple, spatially-separated signal sources. Light from the multiple signal sources is provided to the sensor by passing through a single, non-branching emission pathway. The skilled person will recognize numerous configurations and/or variations of the systems as set forth herein.

In some configurations, provided herein is a multiplexed optical detection system, comprising: a) two or more light sources, in which a first light source is configured to transmit light of a first wavelength, and in which a second light source is configured to transmit light of a second wavelength, in which the first wavelength differs from the second wavelength, b) a stage that is configured to accommodate a fluidic cartridge, c) an objective lens; and d) an optical sensor, in which the optical sensor comprises a first channel and a second channel, in which the first channel and the second channel are spatially separated on the sensor, and in which the first channel is configured to detect the light of the first wavelength, and in which the second channel is configured to detect the light of the second wavelength.

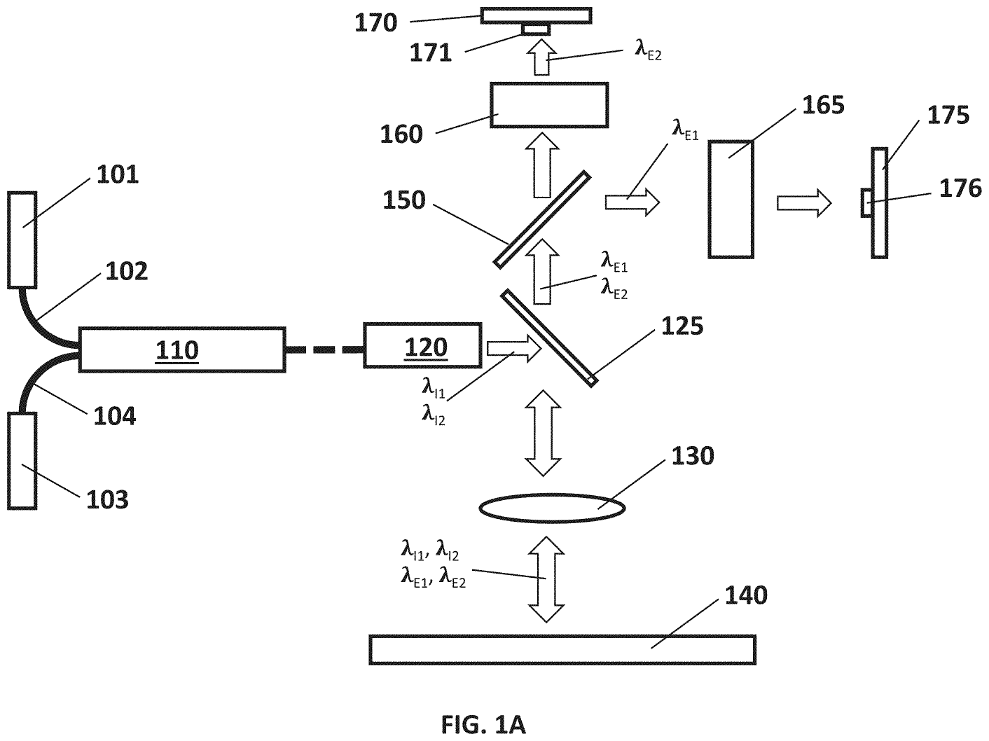

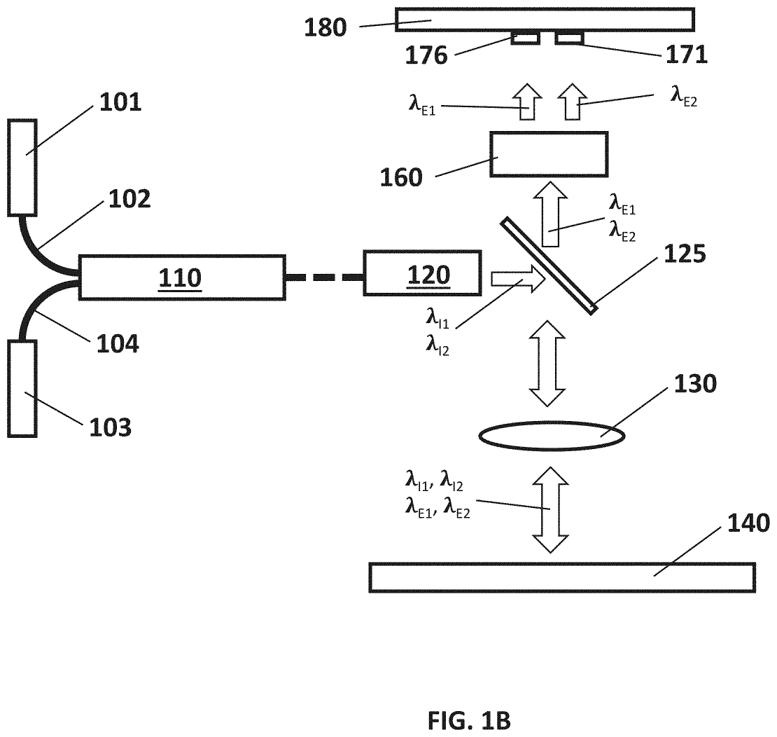

FIGS. 1A-1B illustrate schematic views of multiplexed optical detection systems. FIG. 1A depicts an optical detection system that is configured to detect two signal sources, in which the system contains a first emission pathway for light from a first signal source, and a second emission pathway for light from a second signal source. The system depicted in FIG. 1A contains an illumination pathway with an epi-illumination configuration. A first light source 101 and a second light source 103 (e.g., lamp, laser, light bulb, filament, light-emitting diode, etc.) are optically connected to beam-shaping optics 110 (e.g., filters, polarizing lenses, collimating lenses, beam splitters, etc.) by optional waveguides (e.g., fiberoptic cables, etc.) 102 and 104, respectively. In a first optional configuration, a first illumination beam containing light of wavelength λ_{I1} from the first light source 101 and a spatially-separated second illumination beam containing light of wavelength λ_{I2} from the second light source 103 are transmitted from the beam-combining optics 110, for example by a prismatic beam-splitter. In a second optional configuration, a combined light beam containing light from the first light source 101 and the second light source 103 is formed in a beam-combining optical element 110, and optionally passed through additional beam-shaping optical elements 120. Light from the first light source 101 and the second light source 103 is directed to an illumination target 140 (e.g., a solid support) by contacting an optional mirror 125 (e.g., a dichroic mirror, etc.) and passing through an objective lens 130. After light from the first light source 101 and the second light source 103 contact the illumination target 140, light from a first signal source and light from a second signal source pass through the objective lens 130 and optionally pass through the mirror 125 before entering the branched portion of the emission pathway. Light of wavelength λ_{E2} from the second signal source passes through a beam-splitting element 150 (e.g., a dichroic mirror, a beam splitter, etc.) and optional beam-shaping optics 160 before contacting a second sensor 170 at a second channel 171 that is configured to detect light from the second signal source. Light of wavelength λ_{E1} from the first signal source is redirected by the beam-splitting element 150 and passes through optional beam-shaping optics 165 before contacting a first sensor 175 at a first channel 176 that is configured to detect light from the first signal source.

FIG. 1B depicts an optical detection system that is configured to detect two signal sources, in which the system contains a single emission pathway for light from a first signal source and light from a second signal source. The system depicted in FIG. 1B contains an illumination pathway with an epi-illumination configuration. A first light source of wavelength λ_{I1} 101 and a second light source of wavelength λ_{I2} 103 (e.g., lamp, laser, light bulb, filament, light-emitting diode, etc.) direct light to beam-combining optics 110 (e.g., filters, polarizing lenses, collimating lenses, beam splitters, etc.) by optional waveguides (e.g., fiberoptic cables, etc.) 102 and 104, respectively. The beam-combining optics 110 and optional beam-shaping optics 120 transmit a first illumination beam containing light from the first light source 101 and from a spatially-separated second illumination beam containing light from the second light source 103. The first illumination beam and second illumination beam are directed to an illumination target 140 (e.g., a solid support) by contacting an optional mirror 125 (e.g., a dichroic mirror, etc.) and passing through an objective lens 130. After light from the first light source 101 and the second light source 103 contact the illumination target 140, light from a first signal source and light from a second signal source pass through the objective lens 130 and optionally pass through the mirror 120 before entering optional beam-shaping optics 160. The light of wavelength λ_{E1} from the first signal source contacts a single, multi-channel sensor 180 at a first channel 176 that is disposed on the sensor 180, and light of wavelength λ_{E2} from the second signal source contacts the single, multi-channel sensor 180 at a second channel 171 that is disposed on the sensor 180. The configurations depicted in FIGS. 1A-1B can readily be adapted for trans-illumination of the illumination target 140.

An optical detection system, in some embodiments, is configured with: 1) an illumination pathway that transmits light from one or more light sources to an illumination target; and 2) an emission or transmission pathway that transmits light from one or more signal sources to a sensor that is configured to detect the light from the one or more signal sources. An optical detection system that contains a single, multi-channel sensor, in some embodiments, is configured with: 1) an illumination pathway that transmits light from one or more light sources to an illumination target, in which the transmitted light contacts the illumination target at two or more areas of the illumination target, in which each of the two or more areas comprises at least one signal source; and 2) an emission or transmission pathway that transmits light from the at least two spatially-separated signal sources to a sensor that is configured to detect the light from a first signal source at a first channel on the sensor and light from a second signal source at a second channel on the sensor.

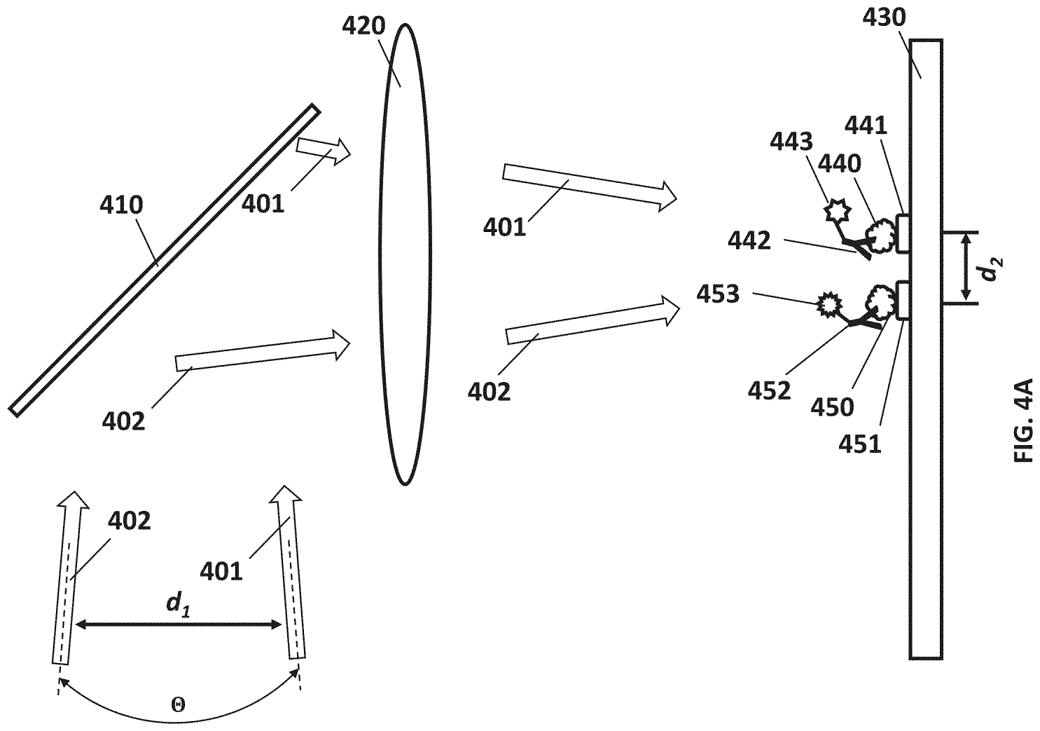

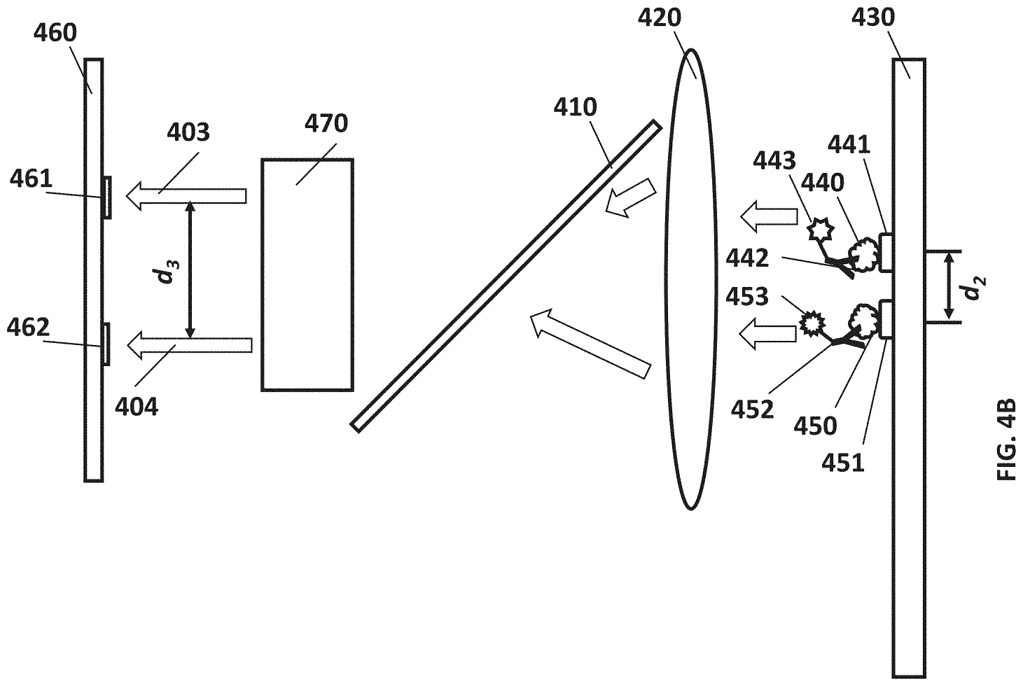

FIGS. 4A-4B illustrate in closer detail the illumination pathway and the emission or transmission pathway of an optical detection system, in accordance with some embodiments. FIG. 4A shows a detailed diagram of components of an illumination pathway that is configured to direct light to two or more signal sources. A first light beam 401 and a spatially-separated second light beam 402, with an angular tilt Θ, are directed toward a dichroic mirror 410 that is configured to redirect the first light beam 401 and the second light beam 402 toward an objective lens 420. The objective lens 420, in some embodiments, is configured to focus light from the first light beam 401 and the second light beam 402 toward an illumination target comprising a solid support 430. The illumination target, in some embodiments, comprises two or more signal sources. FIG. 4A depicts a configuration of an illumination target comprising two spatially separated signal sources. Each signal source comprises a single-analyte (e.g., a polypeptide, a nucleic acid, a cell, etc.), 440 and 450 respectively, that is linked to the solid support 430 by a linking moiety (e.g., a structured nucleic acid particle, an oligonucleotide, a receptor-ligand binding pair, etc.), 451 and 452 respectively. The first single-analyte 440 forms a signal source by binding a first probe 442 (e.g., an antibody, an aptamer, a peptamer, an oligonucleotide, etc.), and the second single-analyte 450 forms a signal source by binding a second probe 452. The first probe 442 comprises a first detectable label 443 that is configured to transmit a signal in the presence of light from the first light beam 401, and the second probe 452 comprises a second detectable label 453 that is configured to transmit a signal in the presence of light from the second light beam 402. The first light beam 401 is separated from the second light beam 402 by a separation distance d₁. The first signal source is separated from the second signal source by a distance d₂, wherein d₁, in some embodiments, is related to d₂ by the relationship d₂=f*Tan(Θ), where f is the focal length of the objective lens. FIG. 4B depicts components of an emission pathway for the illumination target configuration described for FIG. 4A. A first emission light beam 403 is emitted from the detectable label 443 of the first signal source, and a second emission light beam 404 is emitted from the detectable label 453 of the second signal source. The first emission light beam 403 and the second emission light beam 404 pass through the objective lens 420 and the dichroic mirror 410, and optionally additional beam-shaping optics (e.g. a tube lens), then contact a sensor 460. The sensor 460 contains a first channel 461 that is configured to detect light from the first emission light beam 403, and the second channel 462 that is configured to detect light from the second emission light beam 404.

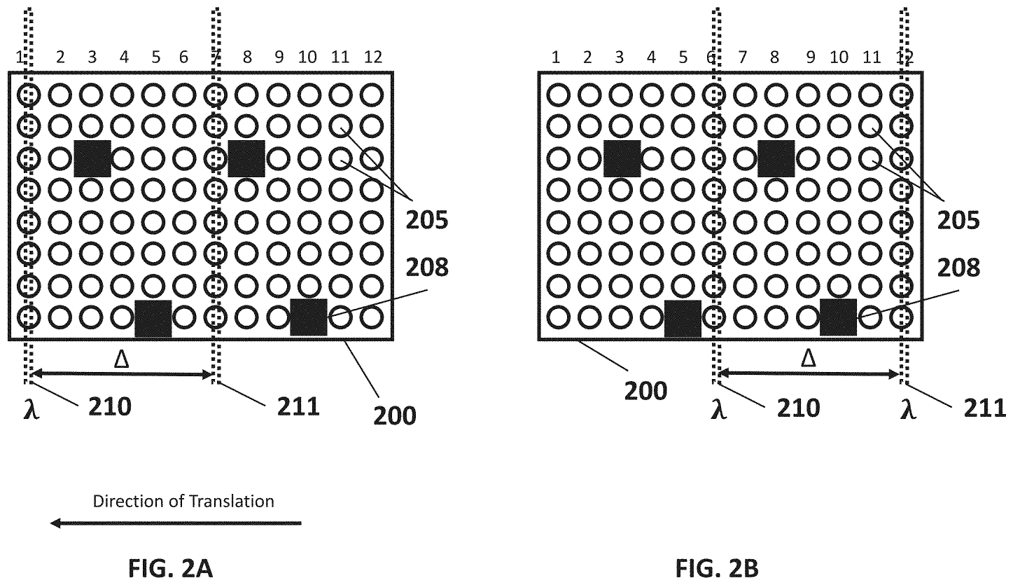

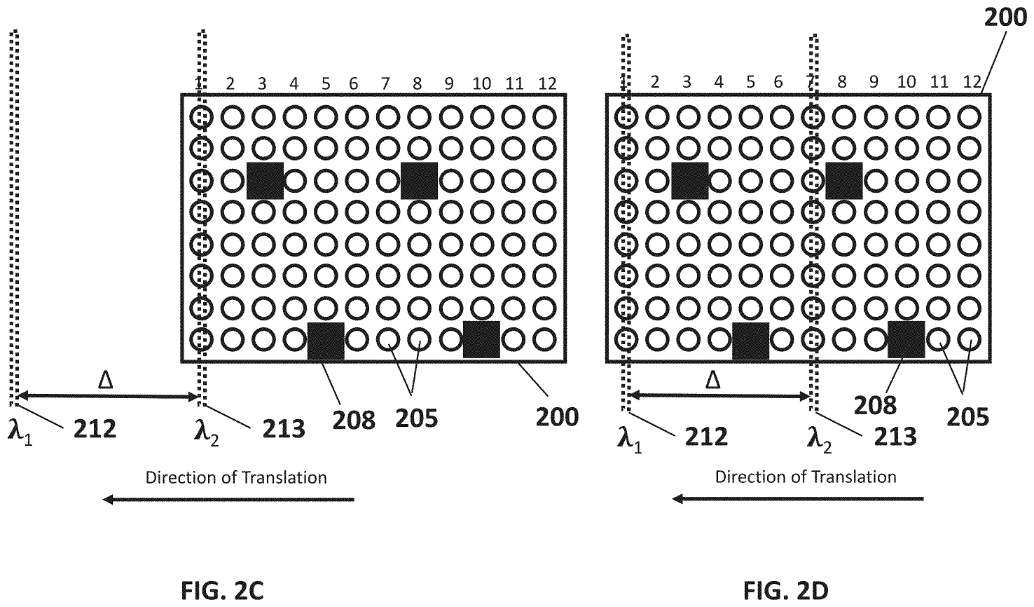

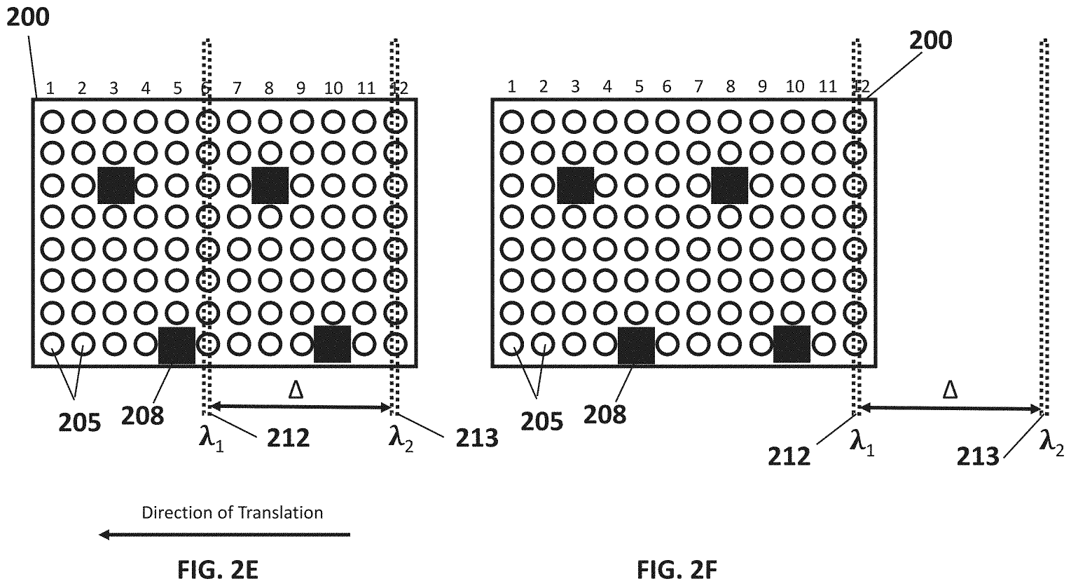

An illumination target, in some embodiments, is simultaneously contacted at two or more spatially-separated regions by light from a single light source or from a plurality of light sources. For example, in a particular configuration, an array containing a plurality of single-analytes, in some embodiments, is contacted in a linescan mode with two lines of light containing light derived from the same light source. FIG. 2A-2B illustrate illumination of a portion of an illumination target (e.g., a single-analyte array) by two spatially-separated lines of light, with each line of light containing light of wavelength λ. The two lines of light are separated by a fixed distance A. FIG. 2A depicts a system containing an illumination target comprising a solid support 200 that contains a patterned rectangular grid of spatial addresses 205 and a plurality of fiducial markings 208 (e.g., for image registration, image alignment, etc.). FIG. 2A depicts the system at a time when a first line of light 210 is illuminating a first subset of the plurality of the rectangular grid of spatial addresses 205 in column 1 and a second line of light 211 is illuminating a second subset of the plurality of the rectangular grid of spatial addresses 205 in column 7 of the patterned array. At the time depicted in FIG. 2A, the solid support 200 is being translated toward the left. FIG. 2B depicts the system at a time when the first line of light 210 is illuminating a third subset of the plurality of the rectangular grid of spatial addresses 205 in column 6 and the second line of light 211 is illuminating a fourth subset of the plurality of the rectangular grid of spatial addresses 205 in column 12 of the patterned array due to the leftward translation of the solid support 200. During the elapsed time between the configuration depicted in FIG. 2A and the configuration depicted in FIG. 2B, each spatial address of the rectangular grid of spatial addresses 205 has been illuminated by at least one of the first line of light 210 or the second line of light 211. The illumination configuration depicted in FIGS. 2A-2B, in some embodiments, is advantageous for decreasing the total scan time of an illumination target by simultaneously illuminating multiple areas of the illumination target.

FIGS. 2C-2F illustrate illumination of a portion of an illumination target (e.g., a single-analyte array) by two spatially-separated lines of light, with a first line of light 212 containing light of wavelength λ₁ and a second line of light 213 containing light of wavelength λ₂. The two lines of light are separated by a fixed distance A. FIG. 2C depicts a system containing an illumination target comprising a solid support 200 that contains a patterned rectangular grid of spatial addresses 205 and a plurality of fiducial markings 208. FIG. 2C depicts the system at a time when the second line of light 213 is illuminating a first subset of the plurality of the rectangular grid of spatial addresses 205 in column 1 and the first line of light 212 is not illuminating the illumination target. At the time depicted in FIG. 2C, the solid support 200 is being translated toward the left. FIG. 2D depicts the system at a time when the first line of light 212 is illuminating the first subset of the plurality of the rectangular grid of spatial addresses 205 in column 1 and the second line of light 213 is illuminating a second subset of the plurality of the rectangular grid of spatial addresses 205 in column 7 of the patterned array due to the leftward translation of the solid support 200. FIG. 2E depicts the system at a time when the first line of light 212 is illuminating the second subset of the plurality of the rectangular grid of spatial addresses 205 in column 6 and the second line of light 213 is illuminating a third subset of the plurality of the rectangular grid of spatial addresses 205 in column 12 of the patterned array due to the leftward translation of the solid support 200. FIG. 2F depicts the system at a time when the first line of light 212 is illuminating the third subset of the plurality of the rectangular grid of spatial addresses 205 in column 12 and the second line of light 213 is no longer illuminating the illumination target. The illumination configuration depicted in FIGS. 2C-2F, in some embodiments, is advantageous for performing multiplexed detection over an entire illumination target by illuminating the entire illumination target with each wavelength of light. A skilled person in the art will readily recognize that for other modes of imaging (e.g., time-delay integration imaging), targets, in some embodiments, are illuminated two-dimensionally. For example, a single-analyte array of any of FIGS. 2A-2E, in some embodiments, is illuminated such that two or more rows and two or more columns of targets are simultaneously illuminated such that an aspect ratio of the illuminated area matches an aspect ratio of a pixel array sensor. In some cases, an illumination area of a single-analyte array, in some embodiments, is configured to be no more than a corresponding aspect of a pixel-based sensor. For example, if a pixel-based sensor contains 20×20 pixels, and each pixel is configured to detect a 50 nm×50 nm region of an array, the corresponding illumination areas would be less than or equal to 1 micron×1 micron. An illumination area of a single-analyte array, in some embodiments, is configured to be less than or equal to an aspect of a pixel sensor to reduce irradiation of non-imaged analytes, thereby reducing photodegradation associated with illumination.

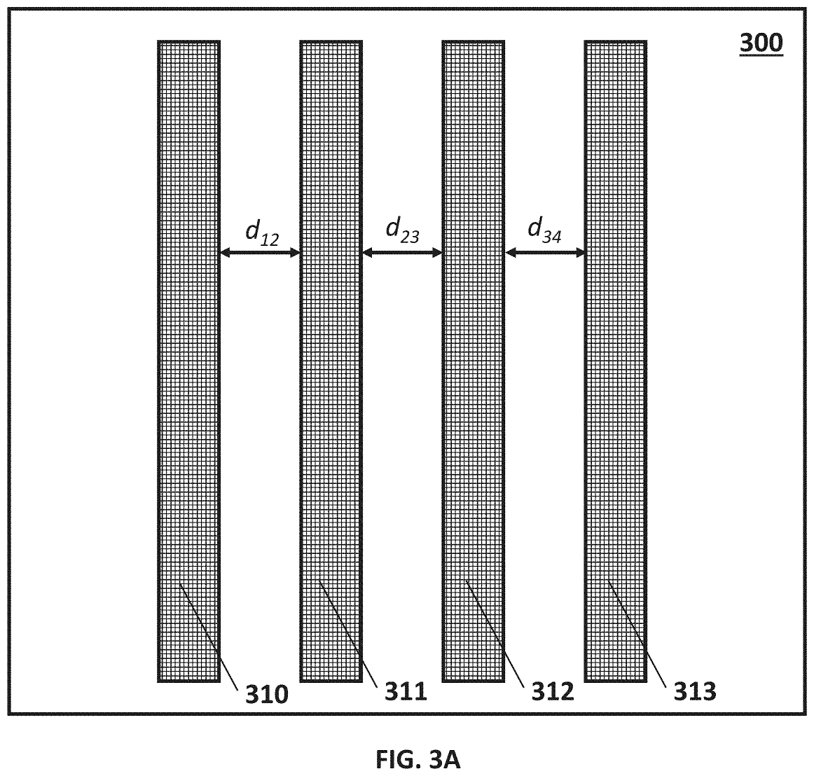

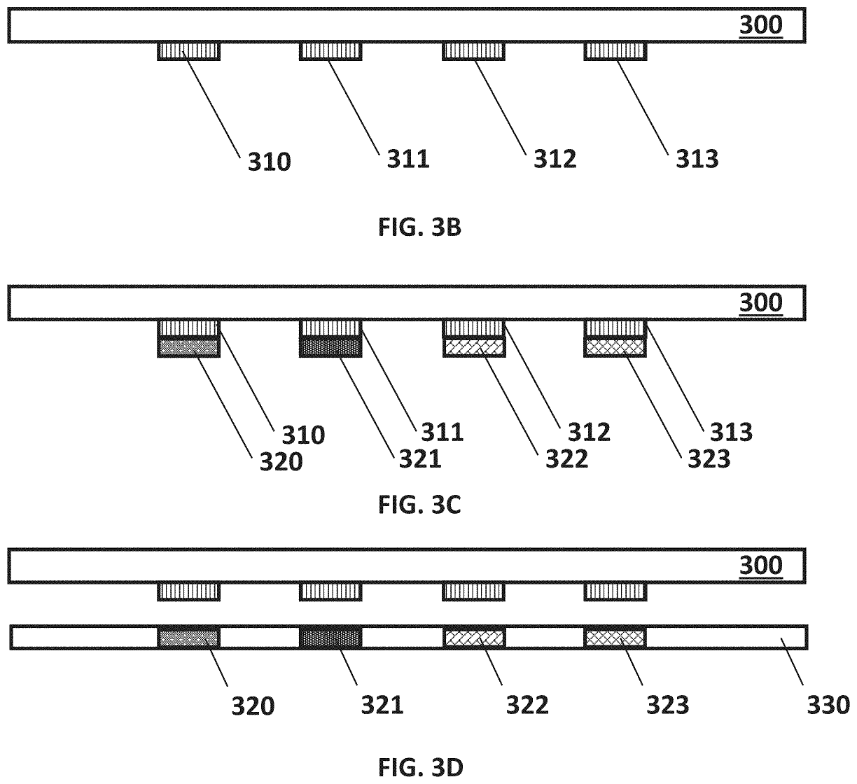

An optical detection system, in some embodiments, comprises a sensor comprising two or more channels. A channel, in some embodiments, comprises an array of light-sensing elements (e.g., a pixel array). In some configurations, a light-sensing element of an array of light-sensing elements comprises a charge-coupled device (CCD) or a complementary metal-oxide semiconductor device (CMOS). FIGS. 3A-3D illustrate various configurations of a multi-channel sensor. FIG. 3A shows a top-down view of a solid support 300 with four channels. Each of the four channels, 310, 311, 312, and 313, respectively, contains an array of light-sensing elements (e.g., CCD, CMOS, FET, etc.), wherein the channels are separated by gaps of distance d₁₂, d₂₃, and d₃₄ respectively. Each channel, in some embodiments, is independently embedded in the solid support 300 or disposed on or adjacent to the solid support 300. FIG. 3B depicts a cross-sectional view of the sensor depicted in FIG. 3A, depicting the four channels (310, 311, 312, 313) disposed on a substantially planar surface of the solid support. FIG. 3C depicts a sensor as shown in FIG. 3B that further comprises optical materials (320, 321, 322, 323 respectively) that are disposed on or adjacent to each channel, in which the optical materials are configured to receive light before passing it to an array of light-sensing elements. FIG. 3D depicts a sensor as shown in FIG. 3B that further comprises optical materials (320, 321, 322, 323 respectively) that are separated from each channel of the sensor. The optical materials, in some embodiments, are disposed on or adjacent to a solid support 330, or incorporated or embedded within the solid support 330. Optical materials (320, 321, 322, 323), in some embodiments, include light filtering materials, light polarizing materials, refractive materials, reflective materials, or a combination thereof. In some configurations, each channel is associated with an optical material that is configured to receive light before the channel. In some configurations, a channel is not associated with an optical material that is configured to receive light before the channel. In some cases, an optical material is associated with a channel to provide a channel a spectral range of detection. For example, a channel comprising a CCD pixel array, in some embodiments, further comprises a deposited filtering material that restricts the channel to detection in a wavelength range from about 600 nanometers (nm) to about 650 nm. In a particular configuration, a filter comprises a CCD-in-CMOS hyperspectral sensor comprising a plurality of detection channels. For example, a sensor containing multiple detection channels can include the IMEC Argus 7 hyperspectral imaging sensor (“CCD-In-CMOS TDI & Multispectral TDI Imaging,” retrieved from https://www.imec-int.com/sites/default/files/imported/CCD-IN-CMOS%2520TDI%2520AND%2520MULTISPECTRAL%2520TDI%2520IMAGING_0.pdf, which is hereby incorporated by reference in its entirety).

An optical detection systems, in some embodiments, comprises a mechanical structure. A mechanical structure, in some embodiments, has one or more functions, including at least one of: 1) securing and/or stabilizing an optical component within the optical detection system; 2) facilitating optical alignment between a first optical component and a second optical component; 3) coupling an optical component to a motion control device (e.g., a translation stage); 4) controlling motion associated with short-period mechanical changes (e.g., vibrational motion) and/or long-period mechanical changes (e.g., thermal motion); and 5) combinations thereof. An optical detection system, in some embodiments, comprises a single mechanical structure. An optical detection system, in some embodiments, comprises a plurality of mechanical structures. An optical detection system, in some embodiments, comprises a plurality of mechanical structures, in which each mechanical structure is configured to couple to at least one other mechanical structure. An optical detection system, in some embodiments, comprises a modular architecture, in which the modular architecture comprises two or more mechanical structures that are configured to couple to at least one other mechanical structure.

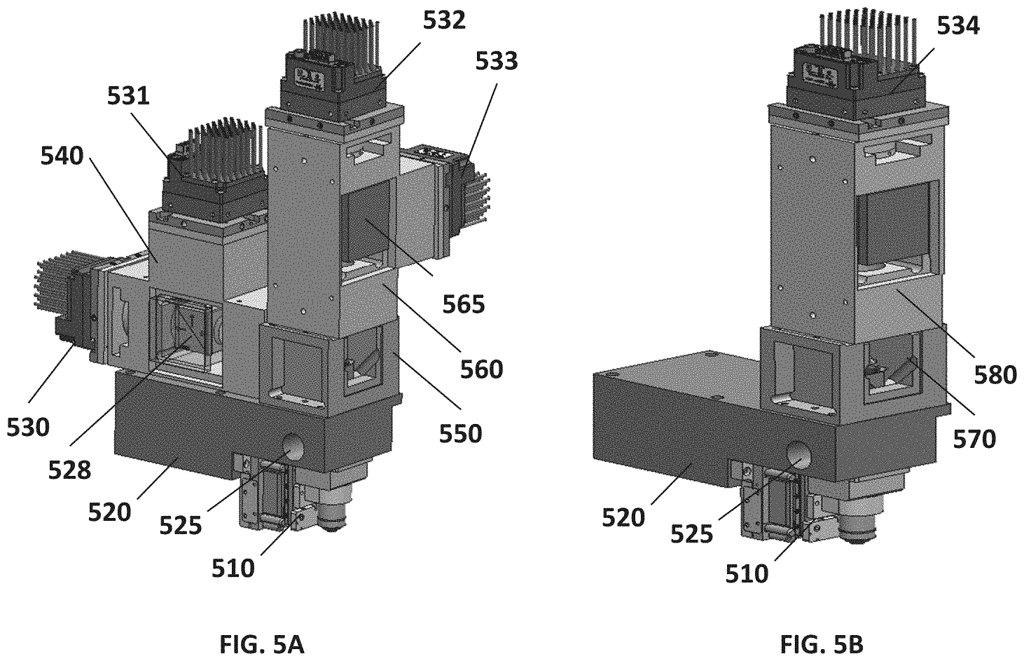

FIGS. 5A-5B illustrate differing configurations of optical detection systems containing modular architecture, in accordance with some embodiments. FIG. 5A depicts a configuration of a portion of an optical detection system containing four single-channel sensors. The optical detection system comprises an objective lens module 510 that is coupled to an illumination pathway module 520 that contains a port 525 that is configured to receive light from one or more light sources. The optical detection system is configured to pass light that is collected by the objective lens 510 through the illumination pathway module 520 to a beam-splitting module 528 that directs a first portion of the light collected by the objective lens 510 to a first single-channel sensor 530 and a second single-channel sensor 531, and directs a second portion of the light collected by the objective lens 510 to a third single-channel sensor 532 and a fourth single-channel sensor 533. The first single-channel sensor 530 and the second single-channel sensor 531 are coupled to the portion of the optical detection system by a first beam-shaping module 540 that incorporates a beam-splitting device 530 that divides received light and passes the light to the first single-channel sensor 530 or the second single-channel sensor 531. The third single-channel sensor 532 and the fourth single-channel sensor 533 are coupled to the portion of the optical detection system by a second beam-shaping module 560 that incorporates a beam-splitting device 565 that divides received light and passes the light to the third single-channel sensor 532 or the fourth single-channel sensor 533. The first beam-shaping module 540 and/or the second beam-shaping module 560, in some embodiments, further comprise additional optical components (not shown in FIG. 5A) that condition received light before passing the light to a sensor, such as collimating lenses, filtering lenses, apertures, etc.

FIG. 5B depicts a configuration of a portion of an optical detection system containing a single multi-channel sensor. The optical detection system comprises an objective lens module 510 that is coupled to an illumination pathway module 520 that contains a port 525 that is configured to receive light from one or more light sources. The optical detection system is configured to pass light that is collected by the objective lens 510 through the illumination pathway module 520 to a first beam-shaping module 570 and an optional second beam-shaping module 580 before passing the collected light to the multi-channel sensor 534. The first beam-shaping module 570 and/or the second beam-shaping module 580, in some embodiments, further comprise additional optical components (not shown in FIG. 5B) that condition received light before passing the light to the multi-channel sensor 534, such as collimating lenses, filtering lenses, apertures, etc.

An optical detection system, in some embodiments, comprises modules in addition to those described in FIGS. 5A-5B. Additional modules, in some embodiments, include an illumination beam generation module (e.g., a laser, a lamp, a light-emitting diode, etc.), an illumination beam-combining module, an illumination beam-shaping module, and an autofocus module. In some embodiments, an illumination beam generation module comprises one or more light sources that are configured to produce light for illumination of an illumination target. An illumination beam-combining module, in some embodiments, is configured to spatially-confine multiple illumination beams (e.g., two or more light beams of differing wavelengths) when transmitting the illumination beams to another module. An illumination beam-shaping module, in some embodiments, is configured to alter one or more properties of a light beam, such as beam coherence, beam focus, collimation, polarization, dispersion, etc. An autofocus module, in some embodiments, is configured to adjust the focal point of light relative to a sensor. An autofocus module, in some embodiments, comprises a translation device that is configured to adjust a position of an optical component (e.g., an objective lens, a focusing lens, an aperture, etc.) relative to a sensor. In some configurations, an autofocus comprises a z-axis translation device.

A modular system, in some embodiments, utilizes one or more fastening elements to couple a first module to a second module. A fastening element, in some embodiments, includes a permanent fastening or a non-permanent fastening element. A permanent fastening element, in some embodiments, comprises a fastening element that only permits separation or de-coupling of a first module from a second module by mechanical destruction of the permanent fastening element. Exemplary permanent fastening elements, in some embodiments, include adhesives, nails, rivets, ties, or combinations thereof. A non-permanent fastening element, in some embodiments, comprises a fastening element that permits reversible coupling and de-coupling of a first module from a second module without mechanical destruction of the fastening element. Exemplary non-permanent fastening elements, in some embodiments, include bolts, screws, anchors, slotted peg systems, ratcheting fasteners, and combinations thereof. In some configurations, a first module comprises a first component of a fastening element (e.g., a peg), and a second module comprises a second component of a fastening element (e.g., a slot), in which the first module is configured to be coupled to the second module by coupling the first component of the fastening element to the second component of the fastening element. In particular configurations, a first module comprises a first component of a fastening element (e.g., a peg), and a second module comprises a second component of a fastening element (e.g., a slot), in which coupling the first component of the fastening element to the second component of the fastening element aligns a component of the first module (e.g., an optical component, a mechanical component, a structural component, an electrical component, a fluidic component) to a component of the second module (e.g., an optical component, a mechanical component, a structural component, an electrical component, a fluidic component).

In some configurations, an optical detection system, as set forth herein, comprises a portion of a larger operational system, such as an analytical instrument. An operational system, in some embodiments, comprises a plurality of subsystems, including an optical detection system and one or more non-optical subsystems. An operational system, in some embodiments, comprises an optical detection system and a non-optical subsystem, in which an operation schedule of the optical detection system is independent of an operation schedule of the non-optical subsystem. For example, an operational system, in some embodiments, comprises an optical detection system, as set forth herein, and a fluidics system, in which the fluidic operation schedule (e.g., fluid transfer, rinsing, mixing, etc.) occurs independently of an optical imaging schedule of the optical detection system (see for example U.S. Provisional Patent Application No. 63/292,676, which is incorporated by reference in its entirety). An operational system, in some embodiments, comprises an optical detection system and a non-optical subsystem, in which an operation schedule of the optical detection system is dependent upon or coupled with an operation schedule of the non-optical subsystem. For example, an operational system, in some embodiments, comprises an optical detection system, as set forth herein, and a motion control system for a single-analyte array, in which a motion control operation schedule (e.g., translating the array during TDI imaging, etc.) is synchronized with an optical imaging schedule of the optical detection system.

An operational system, in some embodiments, comprises an optical detection system, as set forth herein, and one or more non-optical subsystems. A subsystem of the operational system, including the optical detection system or a non-optical subsystem, in some embodiments, comprises a thermally-altering component. A thermally-altering component, in some embodiments, comprises a component that adds or increases thermal energy within the operational system. For example, an operational system, in some embodiments, includes microprocessors, pumps, motion controllers, etc. whose operations produce heat within the operational system. A thermally-altering component, in some embodiments, comprises a component that removes or reduces thermal energy within the operational system. For example, an operational system, in some embodiments, includes a fan, heat exchanger, or air-conditioning device. An operational device, in some embodiments, further comprises an external heat source or heat sink. For example, an operational device, such as a laboratory instrument, in some embodiments, is heated and/or cooled by changes in a temperature environment external to the instrument, radiative heat transfer from external light sources, or heat generation from an adjacent piece of equipment.

An optical detection system, in some embodiments, is disposed within an internal space, cavity, or void of an operational system. An internal space, cavity, or void of an operational system, in some embodiments, is fully or partially enclosed by enclosure material, such as paneling, housing, baffling, etc. An internal space, cavity, or void, in some embodiments, is sealed or unsealed. An internal space, cavity, or void, in some embodiments, is configured to exclude ambient light from contacting an array or other object that is to be detected by the system. An internal space, cavity, or void of an operational system, in some embodiments, comprises an atmosphere. An atmosphere of an internal space, cavity, or void, in some embodiments, is exchanged with an external atmosphere. For example, actuation of a port, door or window of an operational system (e.g., by manual actuation or automated actuation), in some embodiments, causes a partial or complete exchange of an internal atmosphere of the operational system with an external atmosphere. An external atmosphere, in some embodiments, is provided to an operational system by a fluid transfer device. For example, an operational system, in some embodiments, comprises a fan that transfers an external atmosphere into an internal cavity, space, or void of the operational system. In another example, an operational system, in some embodiments, is fluidically connected to a controlled external atmosphere source (e.g., compressed gas such as a chemically inert gas or noble gas) by a mass or volumetric flow device (e.g., a rotameter, mass flow controller, etc.). In some cases, exchange of atmosphere in an internal cavity, space, or void of an operational system, in some embodiments, causes a transient or steady-state temperature differential in the internal cavity, space, or void. A transient or steady-state temperature differential in the internal cavity, space, or void of an operational system, in some embodiments, includes a spatial and/or temporal temperature differential. For example, the opening of a port, door, or window of an operational system, in some embodiments, facilitates an exchange of a warmer internal atmosphere for a cooler external atmosphere, thereby transiently decreasing the temperature in a cavity, space or void of the operational system.

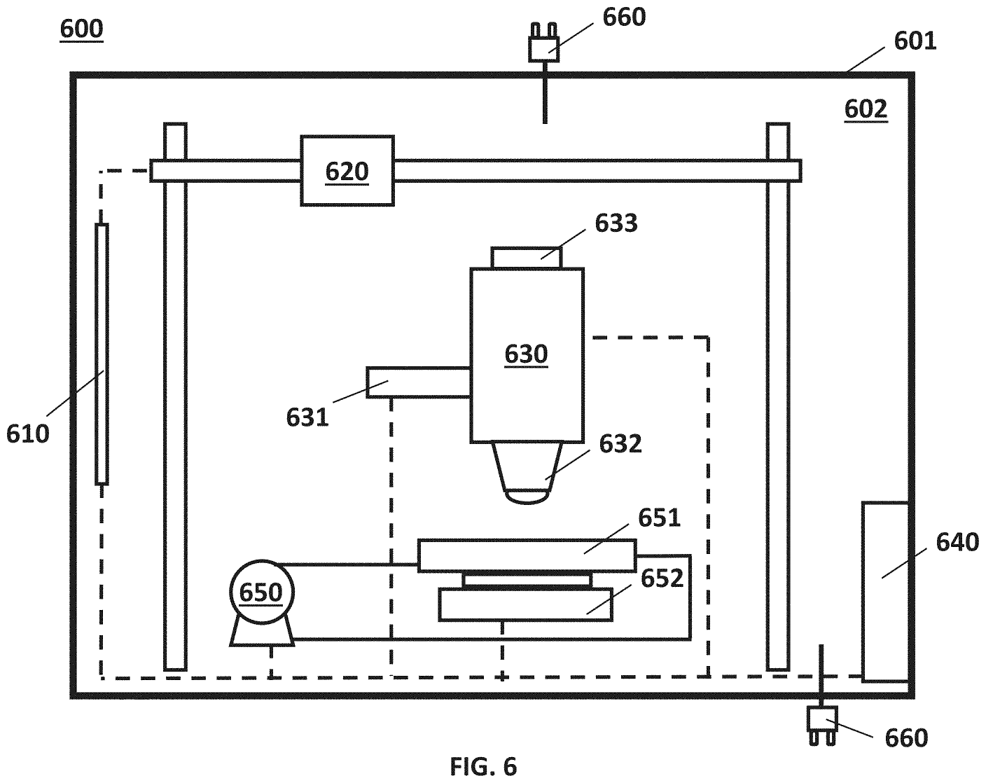

An operational system comprising an optical detection system, in some embodiments, comprises one or more heat-generating devices. A heat-generating device, in some embodiments, conducts operations that generate heat in a transient or steady-state fashion. A heat-generating device, in some embodiments, causes a transient or steady-state temperature differential in an internal cavity, space, or void of an operational system. A transient or steady-state temperature differential in the internal cavity, space, or void of an operational system, in some embodiments, includes a spatial and/or temporal temperature differential. FIG. 6 illustrates an operational system 600 comprising a plurality of thermally-altering components that are enclosed in a space 602 that is surrounded by a housing 601. The plurality of thermally-altering components, in some embodiments, comprises a processor or microprocessor 610 that generates heat while implementing processor-based operations (e.g., running control operations, performing calculations, etc.). The processor or microprocessor 610, in some embodiments, is in communication with (as indicated by dashed lines), and/or in control of, one or more additional thermally-altering components, including a robotic apparatus 620 (e.g., an automated injector, a sample-handling system, etc.), an optical detection system 630 comprising a light source 631, an objective lens 632, and a single-channel or multi-channel sensor 633, a thermal control device 640 (e.g., a fan, a heat exchanger, etc.), and a fluidics system comprising a pump 650, a fluidic cartridge 651, and a motion controller 652. Operational system temperatures or temperature profiles, in some embodiments, is measured by one or more thermocouples 660. In some cases, a component of an optical detection system (e.g., a sensor, an optical device, etc.), in some embodiments, comprises a thermoelectric cooling system that is configured to maintain the component at a temperature below a threshold temperature for operation.

An optical detection system, as set forth herein, in some embodiments, comprises one or more components that undergo a change in a mechanical property (e.g., linear expansion coefficient, volumetric expansion coefficient, Young's modulus, etc.) and/or optical property (e.g., index of refraction, etc.) due to a change in temperature or temperature profile of the optical detection system. In some configurations, a change in temperature or temperature profile of the optical detection system, in some embodiments, is caused by heat generated or removed by one or more thermally-altering components of an operational system comprising the optical detection system. In particular configurations, an optical detection system, in some embodiments, comprises a first component and a second component, in which a change in temperature or temperature profile of the operational system and/or optical detection system alters a mechanical and/or optical property of the first component and the second component, and in which the relative or absolute magnitude of change of the mechanical property for the first component is larger than the relative or absolute magnitude of change of the mechanical property for the second component. For example, an optical detection system, in some embodiments, comprises a first metal structure containing a first metal alloy and a second metal structure containing a second metal alloy, in which a temperature change-induced linear expansion of the first metal structure is larger than a temperature change-induced linear expansion of the second metal structure. In other particular configurations, an optical detection system, in some embodiments, comprises a first component and a second component, in which a non-uniform change in temperature or temperature profile of the operational system and/or optical detection system alters a mechanical and/or optical property of the first component and the second component, and in which the relative or absolute magnitude of change of the mechanical property for the first component is larger than the relative or absolute magnitude of change of the mechanical property for the second component. For example, an optical detection system, in some embodiments, comprises a first metal structure and a second metal structure in which each metal structure comprises the same metal alloy, and in which a temperature change-induced linear expansion of the first metal structure Is larger than a temperature change-induced linear expansion of the second metal structure due to the difference in thermal environment between the first metal structure and the second metal structure.

A change in temperature or temperature profile of an operational system or an optical detection system therein, in some embodiments, causes a change in a detection characteristic (e.g., a focal depth). In some cases, a focusing device, in some embodiments, adjusts an optical detection system characteristic (e.g., z-axis position, working distance, etc.) due to a change in temperature or temperature profile of an operational system or an optical detection system therein. In a particular configuration comprising two or more sensors, in which each sensor detects a unique beam of light, a focusing device, in some embodiments, separately adjusts an optical detection system characteristic for each of the two or more sensors to compensate for differences in a detection characteristic caused by a temperature or temperature profile change of the operational system and/or optical detection system. For example, a multiplexed, four-sensor optical detection system, in which each sensor is configured to detect a unique wavelength of light, in some embodiments, utilizes separate or non-simultaneous autofocusing of light for each sensor due to thermally-induced changes in optimal focal depth for each of the four sensors. Surprisingly, in another particular configuration comprising a single, multi-channel sensor, in which each channel of the single, multi-channel sensor detects a unique beam of light, a focusing device, in some embodiments, simultaneously adjusts an optical detection system characteristic for each unique beam of light to compensate for differences in a detection characteristic caused by a temperature or temperature profile change of the operational system and/or optical detection system. For example, a multiplexed optical detection system comprising a single, four-channel sensor, in which each channel is configured to detect a unique wavelength of light, in some embodiments, comprises an autofocusing device that is configured to simultaneously adjust a focus of light that is directed to each of the four channels to compensate for thermally-induced changes in optimal focal depth for each of the four channels.

An optical detection system, as set forth herein, in some embodiments, experiences a temperature change, for example during a method, as set forth herein, or a step thereof, of at least about 0.1 degree Celsius (° C.), 0.5° C., 1° C., 2° C., 3° C., 4° C., 5° C., 6° C., 7° C., 8° C., 9° C., 10° C., 15° C., 20° C., 25° C., 30° C., or more than 30° C. Alternatively or additionally, an optical detection system, as set forth herein, in some embodiments, experiences a temperature change, for example during a method, as set forth herein, or a step thereof of no more than about 30° C., 25° C., 20° C., 15° C., 10° C., 9° C., 8° C., 7° C., 6° C., 5° C., 4° C., 3° C., 2° C., 1° C., 0.5° C., 0.1° C., or less than 0.1° C. A temperature change, in some embodiments, includes an increase in temperature, a decrease in temperature, or a combination thereof (e.g., a cycling of temperatures could result in a permanent offset of focus in a multi-sensor optical detection system).

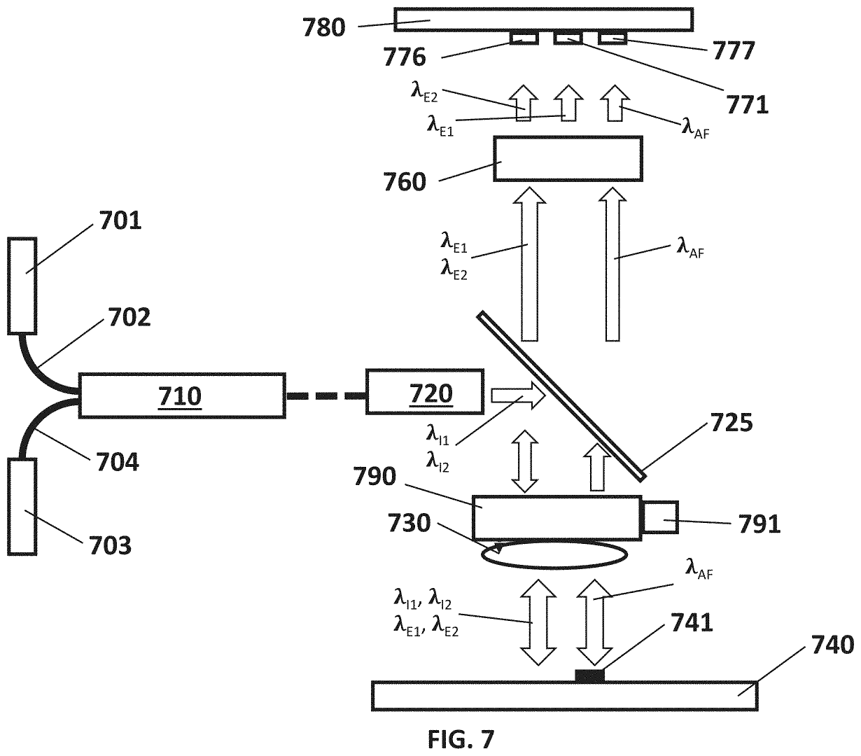

An optical detection system, in some embodiments, comprises a focusing device (e.g., an autofocus device). In some configurations, a focusing device, in some embodiments, comprises a device that is configured to adjust a focal depth, focal distance, or working distance of an optical component (e.g., an objective lens) relative to an illumination target such as an array of single-analytes to be detected. In some configurations, a focusing device comprises a motion control device (e.g., a z-axis translator) that is configured to adjust a distance between a component of an optical detection system (e.g., an objective lens, a sensor) and an illumination target. In some configurations, a focusing device further comprises a light source. A light source of a focusing device, in some embodiments, is configured to provide light of a suitable wavelength or range of wavelengths for focusing an optical detection system. For example, a focusing device, in some embodiments, comprises a source of white light. In some configurations, a light source for a focusing device, in some embodiments, is provided along an illumination pathway that partially or completely differs from an illumination pathway utilized for illumination of an illumination target. FIG. 7 illustrates a schematic of an optical detection system comprising a focusing device 790. A first light source of wavelength λ_{I1} 701 and a second light source of wavelength λ_{I2} 703 (e.g., lamp, laser, light bulb, filament, light-emitting diode, etc.) are optically connected to beam-combining optics 710 (e.g., filters, polarizing lenses, collimating lenses, beam splitters, etc.) by optional waveguides (e.g., fiberoptic cables, etc.) 702 and 704, respectively. The beam-combining optics 710 and optional beam-shaping optics 720 produce a first illumination beam containing light from the first light source 701 and a spatially-separated second illumination beam containing light from the second light source 703. The first illumination beam and second illumination beam are directed to an illumination target 740 (e.g., a solid support) by contacting an optional mirror 725 (e.g., a dichroic mirror, etc.) and passing through an objective lens 730. After light from the first light source 701 and the second light source 703 contact the illumination target 740, light from a first signal source and light from a second signal source pass through the objective lens 730 and optionally pass through the mirror 725 before entering optional beam-shaping optics 760. The light of wavelength λ_{E1} from the first signal source contacts a single, multi-channel sensor 780 at a first channel 771 that is disposed on the sensor 780, and light of wavelength λ_{E2} from the second signal source contacts the single, multi-channel sensor 780 at a second channel 776 that is disposed on the sensor 780. The light from the first signal source and light from the second signal source also pass through a focusing device 790 that comprises a focusing light source 791 (e.g., a light bulb, a laser, a photodiode, etc.). Light of wavelength λ_{AF} from the focusing light source 791 is directed through the focusing device 790 to the illumination target 740. Light from the light source 791 is modified (e.g., by reflection, by fluorescence, by absorption, etc.) by a fiducial element 741 of the illumination target 740 toward the sensor 780 along the same emission or transmission pathway as the light from the first signal source and light from the second signal source. The returned focusing light of wavelength λ_{AF} contacts the sensor 780 at a third channel 777 that is disposed on the sensor 780. Passing light from a light source 791 through a focusing device 790 along a different pathway than light for illuminating an illumination target (e.g., an analyte, a signal source coupled to an analyte, etc.), in some embodiments, simplifies the optical configuration of an optical detection system. For example, a light source for operation of a focusing device, in some embodiments, is configured to not pass through a dichroic mirror or beam splitter. Such a configuration, in some embodiments, is advantageous when utilizing brightfield illumination of a fiducial element for autofocusing, in which the brightfield utilizes a range of wavelengths (e.g., white light) that, in some embodiments, are partially reflected or refracted by a dichroic mirror or beam splitter.

An optical detection system, as set forth herein, in some embodiments, comprises a light source. A light source, in some embodiments, is configured to provide electromagnetic radiation to an illumination target. In some configurations, light from a light source is detected on a sensor of an optical detections system. For example, a sensor, in some embodiments, detects light from a light source reflected by an illumination target to the sensor, or attenuated light caused by absorption of light from the light source by the illumination target. In other configurations, light from a light source, in some embodiments, is utilized to stimulate a signal from a signal source (e.g., fluorescence, luminescence, etc.). A light source, in some embodiments, comprises any conceivable device that is configured to produce a beam of electromagnetic radiation, such as a laser, a lamp, a bulb, a filament, a light-emitting diode (LED), or a combination thereof.

A light source, as set forth herein, in some embodiments, is configured to produce light of with a characteristic wavelength or frequency, or a characteristic range of wavelengths or frequencies. A light source, in some embodiments, is configured to transmit light from the ultraviolet, visible, or infrared region of the electromagnetic spectrum. A light source, in some embodiments, is configured to produce light with a wavelength that can induce a fluorescent or luminescent emission of light from a signal source (e.g., a fluorophore or luminophore). In some configurations, a light source is configured to produce light over a range of wavelengths, in which the range of wavelengths includes a wavelength that can induce a fluorescent or luminescent emission of light from a signal source. In some configurations, a light source is configured to produce light of substantially a single wavelength, in which the single wavelength is a wavelength that can produce a signal from a signal source. A beam of light from a light source, in some embodiments, is considered substantially of a single wavelength if at least about 90%, 91%, 92%, 93%, 94%, 95%, 96%, 97%, 98%, 99%, 99.5%, 99.9%, 99.99%, 99.999%, or more than 99.999% of the light from the light source is of the single wavelength. For example, a light beam, in some embodiments, is considered to be substantially of a wavelength of 550 nanometers (nm) if at least 90% of photons in the light beam have a wavelength of 550 nm.

Light from a light source, in some embodiments, contains light of a characteristic wavelength, such as about 100 nanometers (nm), 150 nm, 200 nm, 250 nm, 300 nm, 350 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 750 nm, 800 nm, 900 nm, 1 micron (μm), 2 μm, 3 μm, 4 μm, 5 μm, 10 μm, or more than 10 μm. Light from a light source, in some embodiments, contains light with a wavelength of at least about 100 nanometers (nm), 150 nm, 200 nm, 250 nm, 300 nm, 350 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 750 nm, 800 nm, 900 nm, 1 micron (μm), 2 μm, 3 μm, 4 μm, 5 μm, 10 μm, or more than 10 μm. Alternatively or additionally, light from a light source, in some embodiments, contains light with a wavelength of no more than about 10 μm, 5 μm, 4 μm, 3 μm, 2 μm, 1 μm, 900 nm, 800 nm, 700 nm, 690 nm, 680 nm, 670 nm, 660 nm, 650 nm, 640 nm, 630 nm, 620 nm, 610 nm, 600 nm, 590 nm, 580 nm, 570 nm, 580 nm, 570 nm, 560 nm, 550 nm, 540 nm, 530 nm, 520 nm, 510 nm, 500 nm, 490 nm, 480 nm, 470 nm, 460 nm, 450 nm, 440 nm, 430 nm, 420 nm, 410 nm, 400 nm, 350 nm, 300 nm, 250 nm, 200 nm, 150 nm, 100 nm, or less than 100 nm.

An optical detection system, as set forth herein, in some embodiments, comprises one or more light pipes. A light pipe, in some embodiments, is configured to transmit light from a first component of an optical detection system to one or more other components of the optical detection system. For example, a light pipe, in some embodiments, is configured to transmit light from a light source to a beam-shaping optical component (e.g., a collimating lens). A light pipe, in some embodiments, is advantageously configured for minimal loss or attenuation of the light beam being transmitted. In some configurations, a light pipe, in some embodiments, comprises a waveguide (e.g., a fiber optic device). In particular configurations, a light pipe, in some embodiments, comprises two or more separated waveguides. For example, a light pipe, in some embodiments, comprises a first fiber optic core and a second fiber optic core, in which the first fiber optic core is configured to transmit light from a first light source and the second fiber optic core is configured to transmit light from a second light source.

In a particular configuration of an optical detection system, a light pipe is configured to transmit light from one or more light sources to a beam-shaping optical device. A beam-shaping optical device, in some embodiments, comprises a beam splitter that is configured to spatially separate a beam of light comprising a first wavelength of light from a beam of light comprising a second wavelength of light. A beam-shaping optical device, in some embodiments, comprises a beam splitter that is configured to spatially separate a beam of light from a first light source from a beam of light from a second light source. Optionally, a beam-shaping optical device, in some embodiments, comprises additional optical components, such as collimating lenses, filtering lenses, polarizing lenses, focusing lenses, expanding lenses, and combinations thereof. In particular configurations, a beam-shaping optical device is configured to produce a first light beam and a second light beam, in which the first light beam is spatially separated from the second light beam. A beam-shaping optical device, in some embodiments, is configured to produce a first light beam and a second light beam, in which the first light beam is spatially separated from the second light beam by a distance that is at least equal to a distance between a first channel and a second channel of a multi-channel sensor. A beam-shaping optical device, in some embodiments, is configured to produce a first light beam and a second light beam, in which the first light beam is spatially separated from the second light beam by a distance that is no more than a distance between a first channel and a second channel of a multi-channel sensor.

An optical detection system, as set forth herein, in some embodiments, comprises a motion control system. A motion control system, in some embodiments, is configured to adjust a position or orientation of a first optical component relative to a second optical component. In a particular configuration, a motion control system comprises a translation system that is configured to adjust a position of a fluidic cartridge relative to a sensor (e.g., a single-channel sensor, a multi-channel sensor, etc.). A motion control system, in some embodiments, is configured to produce motion in any conceivable direction, including translated motion (e.g., along an x-, y-, and/or z-axis), rotational motion (e.g., angular pitch adjustment), and combinations thereof. An optical detection system, in some embodiments, comprises a motion control system that is coupled to a stage that is configured to accommodate a fluidic cartridge. An optical detection system, in some embodiments, comprises a motion control system that is coupled to a sensor. A motion control system, in some embodiments, comprises an x-y motion controller, an x-y-z motion controller, a rotational motion controller, or a combination thereof. In some configurations, a motion control system is configured to produce a motion of the fluidic cartridge that is substantially orthogonal to the first channel or second channel of the sensor.

An optical detection system, as set forth herein, in some embodiments, comprises an objective lens. An objective lens, in some embodiments, is configured to collect light from one or more signal sources and transmit the light to a sensor that is configured to detect the light from the one or more signal sources. An objective lens, in some embodiments, is configured to transmit light from a light source to an illumination target (e.g., an epi-fluorescence microscope configuration, etc.). An objective lens, in some embodiments, comprises one or more lenses in a serial format (e.g., doublet lenses, triplet lenses, etc.). An objective lens be configured to reduce a chromatic and/or spherical aberration (e.g., an achromatic lens, an apochromatic lens, a superachromatic lens, etc.). An objective lens, in some embodiments, is configured to operate when immersed in an imaging fluid (e.g., an air immersion lens, a water immersion lens, an oil immersion lens, etc.). An optical detection system comprising an objective lens, in some embodiments, has a characterized magnification, such as about 5×, 10×, 15×, 20×, 25×, 30×, 35×, 40×, 45×, 50×, 55×, 60×, 65×, 70×, 75×, 80×, 85×, 90×, 95×, 100×, or more than 100×. An optical detection system comprising an lens, in some embodiments, has a characterized magnification, such as at least about 5×, 10×, 15×, 20×, 25×, 30×, 35×, 40×, 45×, 50×, 55×, 60×, 65×, 70×, 75×, 80×, 85×, 90×, 95×, 100×, or more than 100×. Alternatively or additionally, an optical detection system comprising an objective lens, in some embodiments, has a characterized magnification, such as no more than about 100×, 95×, 90×, 85×, 80×, 75×, 70×, 65×, 60×, 55×, 50×, 45×, 40×, 35×, 30×, 25×, 20×, 15×, 10×, 5×, or less than 5×.

An optical detection system, as set forth herein, in some embodiments, comprises a characterized or estimated numerical aperture (e.g., object-space numerical aperture, image-space numerical aperture). An optical detection system, in some embodiments, has a characterized or estimated numerical aperture of at least about 0.001, 0.005, 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, or more than 1.5. Alternatively or additionally, an optical detection system, in some embodiments, comprises a characterized or estimated numerical aperture of no more than about 1.5, 1.45, 1.4, 1.35, 1.3, 1.25, 1.2, 1.15, 1.1, 1.05, 1.0, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.01, 0.005, 0.001, or less than 0.001.

A sensor (e.g., a multi-channel sensor), in some embodiments, is configured for use in a TDI imaging mode. Accordingly, a sensor, in some embodiments, is operated during an imaging process with a pixel shift rate (i.e., a rate at which accumulated charge is transferred from a first pixel to an adjacent second pixel) that is synchronous with a translation rate of a single-analyte array. A pixel shift rate of a sensor, in some embodiments, is related to an array translation rate based upon the intended spatial resolution of a pixel of the sensor. For example, a sensor pixel that is intended to capture a spatial area of 50 nm by 50 nm, in some embodiments, is configured for a pixel shift rate of 20 kilohertz (kHz) for an array translation rate of 1 millimeter per second (mm/s). A sensor, in some embodiments, is configured to operate at a pixel shift rate of at least 1 kHz, 10 kHz, 50 kHz, 100 kHz, 200 kHz, 300 kHz, 400 kHz, 500 kHz, 1000 kHz, or more than 1000 kHz. Alternatively or additionally, a sensor, in some embodiments, is configured to operate at a pixel shift rate of no more than about 1000 kHz, 500 kHz, 400 kHz, 300 kHz, 200 kHz, 100 kHz, 50 kHz, 10 kHz, 1 kHz, or less than 1 kHz. A method, as set forth herein, in some embodiments, comprises operating a sensor at a pixel shift rate set forth herein. In some cases, a sensor is operated during an imaging process with a pixel shift rate that is asynchronous with a translation rate of a single-analyte array. A sensor, in some embodiments, is configured to operate at a pixel shift rate that is at least about 0.001%, 0.01%, 0.1%, 1%, 5%, 10%, 25%, 50%, 75%, 99%, or more than 99% greater or less than an equivalent array translation rate. Alternatively or additionally, a sensor, in some embodiments, is configured to operate at a pixel shift rate that is no more than about 99%, 75%, 50%, 25%, 10%, 5%, 1%, 0.1%, 0.01%, 0.001%, or less than 0.001% greater or less than an equivalent array translation rate.

A single-analyte array, in some embodiments, is translated (e.g., by a translation stage) relative to a sensor at a fixed or variable translation rate. A translation rate of a single-analyte array, in some embodiments, is an average, maximum, or minimum translation rate. A single-analyte array, in some embodiments, is translated at a rate of at least about 0.01 mm/s, 0.1 mm/s, 1 mm/s, 5 mm/s, 10 mm/s, 50 mm/s, 1 centimeter/second (cm/s), 5 cm/s, 10 cm/s, or more than 10 cm/s. Alternatively or additionally, a single-analyte array, in some embodiments, is translated at a rate of no more than about 10 cm/s, 5 cm/s, 1 cm/s, 50 mm/s, 10 mm/s, 5 mm/s, 1 mm/s, 0.1 mm/s, 0.01 mm/s, or less than 0.01 mm/s. A method, as set forth herein, in some embodiments, comprises translating a single-analyte array at an array translation rate, as set forth herein. In some cases, a stage translation rate is determined based upon a maximum, minimum, or optimum pixel shift rate of a sensor.

### Detected Compositions

An optical detection system, as set forth herein, in some embodiments, is utilized to detect a composition on an illumination target. A detected composition, in some embodiments, comprises a single-analyte. A detected composition, in some embodiments, comprises a plurality of single-analytes. A detected composition, in some embodiments, comprises a single-analyte, in which the single-analyte comprises an optically detectable label. A detected composition, in some embodiments, comprises a single-analyte, in which the single-analyte does not comprise an optically detectable label. A detected composition, in some embodiments, comprises a single-analyte complex, in which the complex comprises a single-analyte and a detectable moiety, and in which the detectable moiety is configured to form an interaction (e.g., a binding interaction, a covalent interaction, etc.) with the single-analyte. A detected composition, in some embodiments, comprises a single-analyte complex, in which the complex comprises a single-analyte and a detectable moiety, and in which the detectable moiety is bound by an interaction with the single-analyte. A detected composition, in some embodiments, comprises a single-analyte complex that is stable for the duration of an imaging process by an optical detection system. For example, a single-analyte complex comprising a single-analyte bound to a detectable probe by a streptavidin-biotin linkage would be stable for a time scale that exceeds the time scale of an imaging process (e.g., 1 second, 1 minute, 1 hour, 1 day, etc.). A detected composition, in some embodiments, comprises a single-analyte complex that is not stable for the duration of an imaging process by an optical detection system. For example, a single-analyte complex comprising a single-analyte bound to an affinity agent (e.g., an antibody, an aptamer, etc.), in some embodiments, dissociates within a time scale of an imaging process. It shall be understood that an unstable single-analyte complex, in some embodiments, is still detectable by an optical detection system; rather the likelihood of detection, in some embodiments, is governed by a stochastic process, an equilibrium process, a non-equilibrium process, etc.

An illumination target, as set forth herein, in some embodiments, comprises an array. An array, in some embodiments, comprise a solid support comprising a surface, in which the surface comprises a plurality of single-analyte binding sites. A single-analyte binding site of an array, in some embodiments, is configured to couple a single-analyte. A single-analyte binding site of an array, in some embodiments, is configured to couple no more than one single-analyte. A single-analyte binding site of an array, in some embodiments, is configured to couple more than one single-analyte. An array, in some embodiments, comprises a plurality of single-analyte binding sites, in which each single-analyte binding site of the plurality of single-analyte binding sites is configured to couple a single-analyte. An array, in some embodiments, comprises a plurality of single-analyte binding sites, in which each single-analyte binding site of the plurality of single-analyte binding sites is configured to couple no more than one single-analyte. An array, in some embodiments, comprises a plurality of single-analyte binding sites, in which each single-analyte binding site of the plurality of single-analyte binding sites is configured to couple a plurality of single-analytes. An array, in some embodiments, further comprises one or more interstitial regions. An interstitial region, in some embodiments, comprises a region of a solid support or a surface thereof that is configured to not couple a single-analyte. An interstitial region, in some embodiments, comprises a region of a solid support or a surface thereof that is configured to reduce or minimize non-specific binding of a moiety (e.g., an affinity agent, etc.). An interstitial region, in some embodiments, spatially separates a first single-analyte binding site from a second single-analyte binding site. An interstitial region, in some embodiments, is sufficiently sized to make a first single-analyte at a first single-analyte binding site spatially distinguishable from a second single-analyte at a second single-analyte binding site by an optical detection system, as set forth herein.

An array, in some embodiments, comprises a patterned or ordered array. A patterned or ordered array, in some embodiments, comprises a regular geometry as defined by the location of addresses, for example, addresses that function as single-analyte binding sites, such as a rectangular grid, a circular grid, a triangular grid, or a hexagonal grid. A patterned or ordered array, in some embodiments, comprises a repeated pattern of addresses, for example, addresses that function as single-analyte binding sites. The repeated pattern, in some embodiments, includes at least 2, 3, 4, 5, 6, 7, 8, 9, 10, 25, 50, 100 or more addresses. Alternatively or additionally, the repeated pattern, in some embodiments, includes at most 100, 50, 25, 10, 9, 8, 7, 6, 5, 4, 3, or 2 addresses. One or more addresses, in some embodiments, is formed by a nanofabrication process, such as photolithography, Dip-Pen nanolithography, nanoimprint lithography, nanosphere lithography, nanoball lithography, deep-ultraviolet lithography, nanopillar arrays, nanowire lithography, scanning probe lithography, thermochemical lithography, thermal scanning probe lithography, local oxidation nanolithography, molecular self-assembly, stencil lithography, or electron-beam lithography. A patterned or ordered array, in some embodiments, comprises a raised or indented feature relative to a planar surface of a solid support. In a particular configuration, an array comprises a plurality of raised features (e.g., pillars, posts, etc.), in which each raised feature comprises a substantially planar surface that is configured to couple a single-analyte, and in which each substantially planar surface is coplanar with each other substantially planar surface. In another particular configuration, an array comprises a plurality of indented features (e.g., microwells, nanowells, picowells, etc.), in which each indented feature is configured to couple a single-analyte, and in which each indented feature of the plurality of indented features has substantially the same shape or morphology (e.g., depth, width, diameter, profile, etc.). Additional aspects of array patterning and formation are described in U.S. Pat. No. 11,203,612, U.S. Provisional Patent Applications Nos. 63/256,761 and 63/275,298, and Patent Cooperation Treaty Publication WO 2021087402A1, each of which is incorporated by reference in its entirety.

An address such as a single-analyte binding site, in some embodiments, is a component of a plurality of addresses or single-analyte binding sites. A plurality of addresses or single-analyte binding sites, in some embodiments, has an average spacing or pitch between adjacent addresses or binding sites. An average spacing or pitch, in some embodiments, is measured as an average distance between centerpoints of two adjacent addresses or single-analyte binding sites. A single-analyte array, in some embodiments, comprises a plurality of addresses or single-analyte binding sites with an average spacing or pitch of at least about 10 nanometers (nm), 15 nm, 20 nm, 25 nm, 50 nm, 75 nm, 100 nm, 150 nm, 200 nm, 250 nm, 300 nm, 350 nm, 400 nm, 450 nm, 500 nm, 600 nm, 700 nm, 800 nm, 900 nm, 1 micron (μm), 5 μm, 10 μm, 20 μm, 30 μm, 50 μm, 100 μm, or more than 100 μm. Alternatively or additionally, a single-analyte array, in some embodiments, comprises a plurality of single-analyte binding sites or addresses with an average spacing or pitch of no more than about 100 μm, 50 μm, 30 μm, 20 μm, 10 μm, 5 μm, 1 μm, 900 nm, 800 nm, 700 nm, 600 nm, 500 nm, 450 nm, 400 nm, 350 nm, 300 nm, 250 nm, 200 nm, 150 nm, 100 nm, 75 nm, 50 nm, 25 nm, 20 nm, 15 nm, 10 nm, or less than 10 nm.

A single-analyte array, in some embodiments, comprises a plurality of addresses or single-analyte binding sites. A single-analyte array, in some embodiments, comprises at least about 1×10², 1×10³, 1×10⁴, 1×10⁵, 1×10⁶, 1×10⁷, 1×10⁸, 1×10⁹, 1×10¹⁰, 1×10¹¹, 1×10¹², or more than 1×10¹² single-analyte binding sites or addresses. Alternatively or additionally, a single-analyte array, in some embodiments, comprises no more than about 1×10¹², 1×10¹¹, 1×10¹⁰, 1×10⁹, 1×10⁸, 1×10⁷, 1×10⁶, 1×10⁵, 1×10⁴, 1×10³, 1×10², or less than 1×10² single-analyte binding sites or addresses.

A single-analyte binding site or other address of a single-analyte array, in some embodiments, is configured to couple a single-analyte. A single-analyte binding site of a single-analyte array, in some embodiments, is configured to couple a single-analyte by a non-covalent interaction (e.g., electrostatic interaction, magnetic interaction, receptor-ligand binding, nucleic acid hybridization, hydrogen bonding, van der Waals interactions, etc.). A single-analyte binding site or address of a single-analyte array, in some embodiments, is configured to couple a single-analyte by a covalent interaction (e.g., a Click-type reaction, ligation, etc.).

An address or single-analyte binding site of a single-analyte array, in some embodiments, comprises a linking moiety that is configured to couple a single-analyte to the single-analyte binding site. A linking moiety, in some embodiments, comprises a chemical entity that is configured to control the orientation of a single-analyte at a single-analyte binding site, thereby facilitating detection of the single-analyte. A linking moiety, in some embodiments, comprises a chemical entity that is configured to couple a single-analyte to a single-analyte binding site and is further configured to prevent binding of other chemical entities to the single-analyte binding site. In some configurations, a linking moiety, in some embodiments, comprises a nucleic acid linking moiety. In particular configurations, a linking moiety, in some embodiments, comprises a nucleic acid nanoparticle or a structured nucleic acid particle (e.g., nucleic acid origami, nucleic acid nanoball, etc.). A linking moiety, in some embodiments, comprises a coupling moiety that is configured to form a covalent interaction or a non-covalent interaction with a single-analyte binding site. A linking moiety, in some embodiments, comprises a display moiety that is configured to form a covalent interaction or a non-covalent interaction with a single-analyte. A linking moiety, in some embodiments, is coupled to a single-analyte binding site by a covalent interaction or a non-covalent interaction. A linking moiety, in some embodiments, is coupled to a single-analyte by a covalent interaction or a non-covalent interaction. A single analyte, in some embodiments, is coupled to a linking moiety covalently or non-covalently. Additional aspects of linking moieties are described in U.S. Pat. Nos. 11,203,612 and 11,505,796, each of which is herein incorporated by reference.

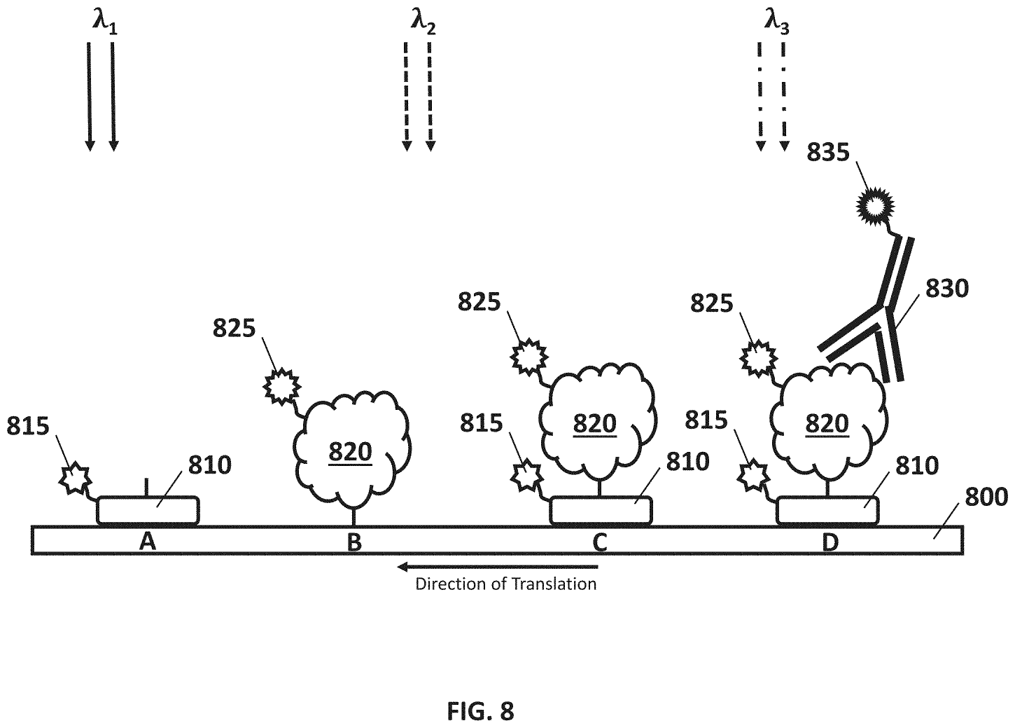

FIG. 8 illustrates a single-analyte array with multiple detectable compositions. Each detectable composition, in some embodiments, is uniquely distinguishable by an optical detection system that is configured for multiplexed detection. FIG. 8 depicts a solid support 800 comprising single-analyte binding sites A-D. Single-analyte binding site A comprises a linking moiety 810 that is coupled to the solid support 800. The linking moiety 810 comprises a first detectable label 815 that emits light in the presence of light of wavelength λ₁. Single-analyte binding site B comprises a single-analyte 820 that is coupled to the solid support 800. The single-analyte 820 comprises a second detectable label 825 that emits light in the presence of light of wavelength λ₂. Single-analyte binding site C comprises a linking moiety 810 that is coupled to a single-analyte 820. The linking moiety 810 is coupled to a first detectable label 815 and the single-analyte is coupled to a second detectable label 825. Single-analyte binding site D comprises a single-analyte complex, in which the single-analyte complex comprises a linking moiety 810, a single-analyte 820 coupled to the linking moiety 810, and an affinity agent 830 coupled to the single-analyte 820. The linking moiety 810 comprises a first detectable label 815, the single-analyte 820 comprises a second detectable label 825, and the affinity agent 830 comprises a third detectable label 835 that emits light in the presence of light of wavelength λ₃.

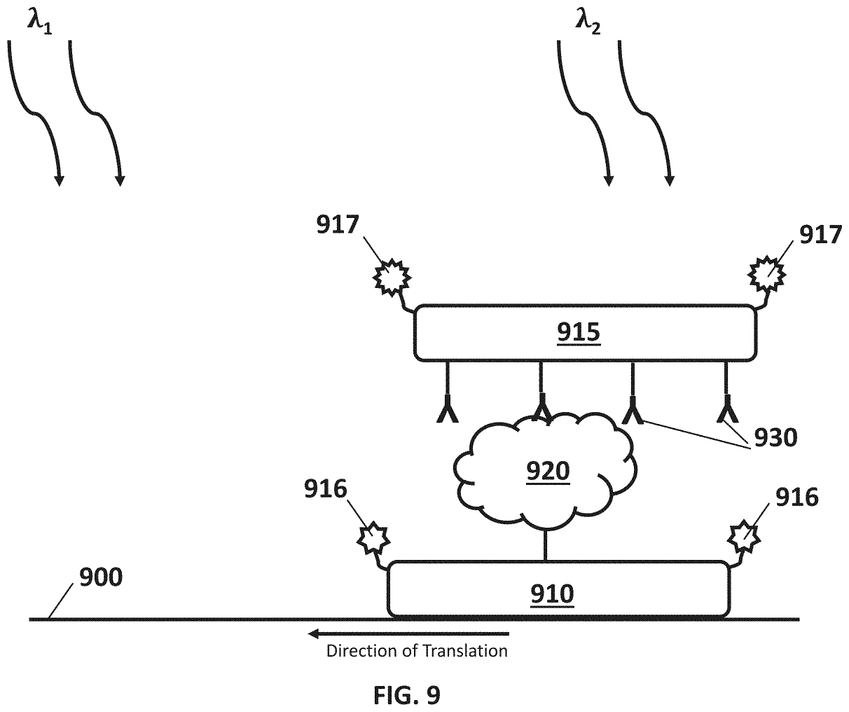

FIG. 9 illustrates a particular configuration of a detectable single-analyte composition that, in some embodiments, is detected by an optical detection system, as set forth herein. A single-analyte 920 is coupled to a single-analyte binding site of a solid support 900 by a linking moiety 910 (e.g., a nucleic acid origami). The linking moiety 910 comprises a first plurality of detectable labels 916 that emit light in the presence of light of wavelength λ₁. The single-analyte 920 forms a binding interaction with a detectable probe that comprises a retaining moiety 915 (e.g., a nucleic acid nanoparticle, a nucleic acid origami, a nanoparticle), a plurality of affinity agents 930 (e.g., antibodies, antibody fragments, aptamers, peptamers, etc.), and a second plurality of detectable labels 917 that emit light in the presence of light of wavelength λ₂. Additional aspects of detectable probes and retaining moieties 915 are set forth in U.S. Patent Publication No. 20220162684A1, which is herein incorporated by reference in its entirety.

An optical detection system, as set forth herein, in some embodiments, is configured to detect a single-analyte composition such as the one depicted in FIG. 9. In some cases, a single analyte, in some embodiments, is considered detected by a detectable probe if the single analyte or a linking moiety attached thereto can be identified as co-localized at an address at which the detectable probe is detected. Accordingly, an optical detection system, in some embodiments, is configured to have similar detection efficiencies for light of a first wavelength λ₁ and light of a second wavelength λ₂. For example, an optical detection system, in some embodiments, comprises a first optical pathway that is optimized for light of a first wavelength λ₁ and a second optical pathway that is optimized for light of a second wavelength λ₂. In another example, an optical detection system, in some embodiments, comprises a multi-channel sensor that is configured to detect light of a first wavelength λ₁ and light of a second wavelength λ₂ with similar quantum efficiencies. An optical detection system, in some embodiments, is considered to detect light of a first wavelength λ₁ and light of a second wavelength λ₂ similarly if the detection quantum efficiencies differ by no more than about 50%, 40%, 30%, 20%, 10%, 5%, or less than 5%. In some configurations, an optical detection system, in some embodiments, is configured to detect co-localized detectable labels simultaneously. For example, a multi-sensor optical detection system such as the system depicted in FIG. 5A, in some embodiments, is configured to simultaneously detect light of a first wavelength λ₁ corresponding to a first detectable label, and light of a second wavelength λ₂ corresponding to a second detectable label. In other configurations, an optical detection system, in some embodiments, is configured to detect co-localized detectable labels sequentially. For example, FIG. 9 depicts spatial separation of light of a first wavelength λ₁ and light of a second wavelength λ₂, thereby promoting detection of the second detectable label at a first time and detection of the first detectable label at a later second time (based upon the direction of translation of the single-analyte composition shown).

A detectable composition, in some embodiments, comprises a single-analyte, such as a cell, a polypeptide, a nucleic acid, a metabolite, a nanoparticle, or a combination thereof. A detectable composition, in some embodiments, comprises a single-analyte that is derived from a sample. A sample, in some embodiments, is derived from an organism or an organism-derived substance or material. A sample, in some embodiments, comprises any type of organism, including animals, non-human animals, humans, plants, fungi, bacteria, protozoa, archaea, viruses, or combinations thereof. The organism, in some embodiments, is a domesticated, modified, or engineered organism, such as poultry, livestock, genetically-modified crops, non-modified crops, transgenic animals, transgenic plants, or production strains of microorganisms (e.g., E. coli, S. cerevisiae). A sample, in some embodiments, comprises a substance derived from an organism, such as an extracellular secretion or debris from a deceased cell. A sample, in some embodiments, is collected from a 2D cell culture line, a 3D cell culture line, a plant tissue sample, an animal tissue sample, a non-human animal tissue sample, a fungal tissue sample, a cultured tissue sample, a human patient-derived tissue sample, a veterinary patient-derived tissue sample, a skin or tissue swab, a tissue biopsy sample, a bodily fluid sample (e.g., blood plasma, blood serum, whole blood, urine, cerebrospinal fluid, saliva, semen, vaginal secretions, tears, mucus), a fecal sample, a cellular lysate, a fixed tissue sample (e.g., FFPE), a single-cell organism, a tissue-derived single cell, a secreted sample, an environmental sample, a microbial sample, a microbiome sample, a biofilm sample, or a non-biological sample.

A detectable composition, or a plurality of detectable compositions, in some embodiments, is bound to a solid support to form a single-analyte array. In some configurations, a solid support of a single-analyte array further comprises an organic layer or coating. In particular configurations, an organic layer or coating comprises a passivating moiety. A passivating moiety, in some embodiments, is configured to reduce a likelihood of non-specific binding of a molecule to a solid support. For example, a passivating moiety, in some embodiments, comprises a hydrophobic moiety, a hydrophilic moiety, a polar moiety, or a non-polar moiety. In particular configurations, a passivating moiety comprises a linear polyethylene glycol (PEG) moiety, a branched PEG moiety, a linear alkane moiety, a branched alkane moiety, a linear fluorinated hydrocarbon, a branched fluorinated hydrocarbon, or a combination thereof. In particular configurations, an organic layer or coating comprises a coupling moiety. A coupling moiety, in some embodiments, is configured to couple a single-analyte to the solid support. In particular configurations, a coupling moiety couples a single-analyte to a solid support by a covalent interaction. For example, a coupling moiety, in some embodiments, comprises a Click-type reactive moiety (e.g., dibenzocyclooctylene, azide, transcyclooctene, methyl tetrazine, etc.). In other particular configurations, a coupling moiety is configured to couple a single-analyte to a solid support by a non-covalent interaction. For example, a coupling moiety, in some embodiments, comprises an oligonucleotide, an electrically-charged moiety, a magnetic moiety, or a member of a receptor-ligand binding pair (e.g., streptavidin-biotin, SpyCatcher-SpyTag, SnoopCatcher-SnoopTag, SdyCatcher-SdyTag, etc.).

A single-analyte array comprising a detectable single-analyte composition or a plurality of single-analyte compositions, in some embodiments, is disposed within a fluidic cartridge or flow cell. A fluidic cartridge, in some embodiments, comprises an internal volume, wherein a single-analyte array is disposed within the internal volume. A fluidic cartridge or flow cell, in some embodiments, comprises one or more components that are joined to form the fluidic cartridge or flow cell. For example, a fluidic cartridge, in some embodiments, is formed by joining a first component comprising fluidic channels and an open fluidic compartment with a second component comprising a nanostructured surface, in which the nanostructured surface is disposed within the fluidic compartment when the fluidic cartridge is joined. A fluidic cartridge or flow cell, in some embodiments, comprises one or more materials, such as metals, metal oxides, polymers, semiconductors, glasses, composites, and combinations thereof. A fluidic cartridge or flow cell, in some embodiments, incorporates an optical coating material, such as a reflective coating, an anti-reflective coating, a filter coating, a polarizing coating, a dielectric coating, or a combination thereof. A material chosen for a component of a fluidic cartridge or flow cell, in some embodiments, has an index of refraction of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, or more than 4.0. A material chosen for a component of a fluidic cartridge or flow cell, in some embodiments, has an index of refraction of at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, or more than 4.0. Alternatively or additionally, a material chosen for a component of a fluidic cartridge or flow cell, in some embodiments, has an index of refraction of no more than about 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, or less than 0.1.

A detectable single-analyte composition, in some embodiments, comprises a signal source. A signal source, in some embodiments, comprises one or more detectable labels configured to produce a detectable signal. The detectable labels, in some embodiments, are the same as each other or, in some embodiments, differ from each other. In particular configurations, a detectable signal, in some embodiments, comprises an optically detectable signal, such as a photon of light. A signal source, in some embodiments, comprises a detectable label such as a fluorophore, luminophore, or radiolabel. A signal source, in some embodiments, comprises a detectable label that is detectable by a method such as fluorescence measurement, luminescence measurement, fluorescence lifetime measurement, luminescence lifetime measurement, or scintillation counting. In some configurations, a single-analyte array comprises a first signal source and a second signal source, in which the first signal source comprises a first detectable label (e.g., a fluorophore, a luminophore, etc.) and the second signal source comprises a second detectable label (e.g., a differing fluorophore, a differing luminophore, etc.). In some configurations, a single-analyte array comprises a first fluorescent signal source and a second fluorescent signal source, in which the first fluorescent signal source comprises a first fluorophore with a first peak absorption wavelength and the second fluorescent signal source comprises a second fluorophore with a second peak absorption wavelength. In some configurations, a single-analyte array comprises a first fluorescent signal source and a second fluorescent signal source, in which the first fluorescent signal source comprises a first fluorophore with a first peak emission wavelength and the second fluorescent signal source comprises a second fluorophore with a second peak emission wavelength. A peak emission wavelength, in some embodiments, is characterized as a wavelength of light released from a fluorophore due to an absorption of a photon of light by the fluorophore. In particular configurations, a first fluorophore comprises a same peak absorption wavelength as a second fluorophore. In particular configurations, a first fluorophore comprises a same peak emission wavelength as a second fluorophore. In particular configurations, a first fluorophore does not comprise a same peak absorption wavelength as a second fluorophore. In particular configurations, a first fluorophore does not comprise a same peak emission wavelength as a second fluorophore. In some configurations, a first fluorophore and a second fluorophore comprise a same chemical structure. In other configurations, a first fluorophore and a second fluorophore comprise a differing chemical structure. In some configurations, a first fluorophore and a second fluorophore are structural variants of a family of fluorophores (e.g., two differing Atto dyes, two differing fluorescein dyes, two differing rhodamine dyes, etc.). In some configurations, a first fluorophore and a second fluorophore are not structural variants of a family of fluorophores.

A detectable signal source, in some embodiments, comprises an agent that is configured to be coupled to a single-analyte (e.g., an affinity agent, a reactive agent, etc.). In some configurations, a detectable signal source comprises an affinity agent, in which the affinity agent is configured to be non-covalently coupled to a single-analyte. In particular configurations, a detectable signal source comprises an affinity agent, in which the affinity agent is configured to be non-covalently coupled to a single-analyte, and in which the affinity agent is configured to be separated from a single-analyte. For example, an affinity agent, in some embodiments, is configured to be coupled to a single-analyte in a first fluidic medium, and de-coupled from the single-analyte in a second fluidic medium. A particularly useful affinity agent composition, in some embodiments, comprises an affinity agent coupled to a fluorescently-labeled nanoparticle. In some configurations, a fluorescently-labeled nanoparticle comprises: i) one or more affinity agents (e.g., antibodies, aptamers, peptamers, etc.), ii) a nanoparticle that is coupled to the one or more affinity agents, and iii) one or more fluorescent or luminescent moieties that are coupled to the fluorescently-labeled nanoparticle (e.g., coupled to the one or more affinity agents, coupled to the nanoparticle, etc.). Useful fluorescently-labeled nanoparticle systems are described in PCT/US2021/058851, which is hereby incorporated by reference in its entirety. A useful affinity reagent composition, in some embodiments, comprises a plurality of fluorescent and/or luminescent moieties. In some configurations, a useful affinity reagent composition comprises a plurality of fluorescent and/or luminescent moieties that produce a signal that is resolvable above a background signal, for example by at least a factor of 1.5, 2, 3, 4, 5, 10, 20, 50, 100, or more than 100, etc. In some configurations, a useful affinity reagent composition comprises a plurality of fluorescent and/or luminescent moieties that produce a signal that is resolvable above a background signal for at least a minimum number of illumination/emission cycles, such as at least about 10, 20, 50, 100, 200, 300, 500, 1000, or more than 1000 illumination/emission cycles. A useful affinity agent composition, in some embodiments, comprises a plurality of affinity agents coupled to a nanoparticle. In some configurations, an affinity agent composition is configured to form a multi-valent binding interaction, in which two or more affinity agents of the affinity agent composition are configured to couple to a single-analyte. A useful affinity agent composition, in some embodiments, comprises a nanoparticle that is configured to couple one or more affinity agents and/or one or more fluorescent and/or luminescent moieties. In some configurations, an affinity agent composition comprises a nanoparticle that is configured to provide tunable orientations of coupled moieties (e.g., affinity agents, fluorescent moieties, luminescent moieties, etc.). In particular configurations, a nanoparticle comprises a nucleic acid nanostructure (e.g., a nucleic acid origami, a nucleic acid nanoball, a nucleic acid nanotube, etc.). In other particular configurations, a nanoparticle comprises a non-nucleic acid nanoparticle (e.g., a fluorescently-labeled polymer, a quantum dot, a carbon nanoparticle, etc.).

A detectable single-analyte composition, in some embodiments, comprises one or more detectable label (e.g., a fluorophore, a luminophore, etc.) that is coupled to a single-analyte. A detectable label, in some embodiments, is directly coupled to a single-analyte (e.g., by a covalent or non-covalent linkage to the single-analyte). A detectable label, in some embodiments, is coupled to a single-analyte by a linking moiety (e.g., a bifunctional linker), in which the linking moiety couples the single-analyte to the single-analyte array. In some configurations, one or more detectable label is coupled to an affinity agent. A detectable label, in some embodiments, is directly coupled to an affinity agent. A detectable label, in some embodiments, is coupled to an affinity agent by a linking moiety.

A fluorophore, in some embodiments, has a characteristic peak absorption wavelength, such as about 100 nanometers (nm), 150 nm, 200 nm, 250 nm, 300 nm, 350 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 750 nm, 800 nm, 900 nm, 1 micron (μm), 2 μm, 3 μm, 4 μm, 5 μm, 10 μm, or more than 10 μm. A fluorophore, in some embodiments, has a characteristic peak absorption wavelength of at least about 100 nanometers (nm), 150 nm, 200 nm, 250 nm, 300 nm, 350 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 750 nm, 800 nm, 900 nm, 1 micron (μm), 2 μm, 3 μm, 4 μm, 5 μm, 10 μm, or more than 10 μm. Alternatively or additionally, a fluorophore, in some embodiments, has a characteristic peak absorption wavelength of no more than about 10 μm, 5 μm, 4 μm, 3 μm, 2 μm, 1 μm, 900 nm, 800 nm, 700 nm, 690 nm, 680 nm, 670 nm, 660 nm, 650 nm, 640 nm, 630 nm, 620 nm, 610 nm, 600 nm, 590 nm, 580 nm, 570 nm, 580 nm, 570 nm, 560 nm, 550 nm, 540 nm, 530 nm, 520 nm, 510 nm, 500 nm, 490 nm, 480 nm, 470 nm, 460 nm, 450 nm, 440 nm, 430 nm, 420 nm, 410 nm, 400 nm, 350 nm, 300 nm, 250 nm, 200 nm, 150 nm, 100 nm, or less than 100 nm. A fluorophore, in some embodiments, has a characteristic peak emission wavelength, such as about 100 nanometers (nm), 150 nm, 200 nm, 250 nm, 300 nm, 350 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 750 nm, 800 nm, 900 nm, 1 micron (μm), 2 μm, 3 μm, 4 μm, 5 μm, 10 μm, or more than 10 μm. A fluorophore, in some embodiments, has a characteristic peak emission wavelength of at least about 100 nanometers (nm), 150 nm, 200 nm, 250 nm, 300 nm, 350 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 750 nm, 800 nm, 900 nm, 1 micron (μm), 2 μm, 3 μm, 4 μm, 5 μm, 10 μm, or more than 10 μm. Alternatively or additionally, a fluorophore, in some embodiments, has a characteristic peak emission wavelength of no more than about 10 μm, 5 μm, 4 μm, 3 μm, 2 μm, 1 μm, 900 nm, 800 nm, 700 nm, 690 nm, 680 nm, 670 nm, 660 nm, 650 nm, 640 nm, 630 nm, 620 nm, 610 nm, 600 nm, 590 nm, 580 nm, 570 nm, 580 nm, 570 nm, 560 nm, 550 nm, 540 nm, 530 nm, 520 nm, 510 nm, 500 nm, 490 nm, 480 nm, 470 nm, 460 nm, 450 nm, 440 nm, 430 nm, 420 nm, 410 nm, 400 nm, 350 nm, 300 nm, 250 nm, 200 nm, 150 nm, 100 nm, or less than 100 nm.

A system, in some embodiments, comprises detectable single-analyte compositions comprising a first fluorophore and a second fluorophore. A first fluorophore and a second fluorophore, in some embodiments, have differing peak absorption wavelengths. A first fluorophore and a second fluorophore, in some embodiments, have differing peak emission wavelengths. A first fluorophore and a second fluorophore, in some embodiments, do not have differing peak absorption wavelengths. A first fluorophore and a second fluorophore, in some embodiments, do not have differing peak emission wavelengths. A first fluorophore and a second fluorophore, in some embodiments, have peak absorption or emission wavelengths that differ by at least about 1 nm, 5 nm, 10 nm, 15 nm, 20 nm, 25 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 225 nm, 250 nm, 300 nm, 400 nm, 500 nm, or more than 500 nm. Alternatively or additionally, a first fluorophore and a second fluorophore, in some embodiments, have peak absorption or emission wavelengths that differ by no more than about 500 nm, 400 nm, 300 nm, 250 nm, 225 nm, 200 nm, 190 nm, 180 nm, 170 nm, 160 nm, 150 nm, 140 nm, 130 nm, 120 nm, 110 nm, 100 nm, 90 nm, 80 nm, 70 nm, 60 nm, 50 nm, 40 nm, 30 nm, 25 nm, 20 nm, 15 nm, 10 nm, 5 nm, 1 nm, or less than 1 nm.

An optical detection system, in some embodiments, is configured to reduce and/or minimize photodegradation phenomena. Photodegradation phenomena, in some embodiments, include processes such as photobleaching, photolysis, photocatalysis, and radical reactions. Photodegradation, in some embodiments, inhibits detection in an optical detection system in one or more ways, including degrading or removing the ability of a detectable label to produce a detectable signal, and damaging a single analyte in a manner that prevents detection of the single analyte. For example, a single analyte, in some embodiments, becomes truncated by a photo-induced cleavage reaction. An optical detection system, in some embodiments, is configured to inhibit photodegradation phenomena by balancing of one or more design parameters, including: i) choice of detectable label (e.g., based on quantum yield, based on absorption wavelength, based on emission wavelength, based on achievable fluorophore density, based on quenching characteristics); ii) choice of excitation source (e.g., laser power density, laser emission spectrum, laser peak wavelength), iii) choice of optical sensor (e.g., based on quantum efficiency as a function of emission wavelength, based on scan rate); and iv) choice of medium in contact with a single analyte.

An optical sensor for an optical detection system, in some embodiments, comprises a back-thinned sensor (e.g., a back-thinned CMOS sensor, a back-thinned CCD device). A back-thinned sensor, in some embodiments, comprises a semiconductive backing material (e.g., silicon) with a reduced thickness at regions of the sensor that contact the light-sensing devices. A semiconductive backing material of a back-thinned sensor, in some embodiments, comprises a thickness of no more than about 200 μm, 100 μm, 50 μm, 25 μm, 10 μm, or less than 10 μm at a region of the sensor that contacts a light-sensing device. Alternatively or additionally, a semiconductive backing material of a back-thinned sensor, in some embodiments, comprises a thickness of at least about 10 μm, 25 μm, 50 μm, 100 μm, 200 μm, or more than 200 μm at a region of the sensor that contacts a light-sensing device. In some configurations, a back-thinned sensor, in some embodiments, is configured in an optical detection system for front-side illumination. In other configurations, a back-thinned sensor is configured in an optical detection system for back-side illumination.

An optical sensor, as set forth herein, in some embodiments, is characterized by a quantum efficiency for detection of light of a particular wavelength, or an average quantum efficiency for a range of wavelengths (e.g., about 400 nm to about 700 nm). A sensor, in some embodiments, is chosen based upon a quantum efficiency at a wavelength of a detectable label that, in some embodiments, is utilized in an optical detection system. An optical sensor, in some embodiments, has a quantum efficiency of at least about 0.5, 0.6, 0.7, 0.8, 0.85, 0.9, 0.95, 0.99, 0.999, or more than 0.999 for a particular wavelength of light. Alternatively or additionally, an optical sensor, in some embodiments, has a quantum efficiency of no more than about 0.999, 0.99, 0.95, 0.9, 0.85, 0.8, 0.7, 0.6, 0.5, or less than 0.5.

An optical detection system, as set forth herein, in some embodiments, comprises a fluidic medium that is contacted with a single analyte. For example, a fluidic cartridge comprising a single analyte or an array comprising a plurality of single analytes, in some embodiments, is charged with a fluidic medium that contacts the single analyte or the array thereof. The fluidic medium, in some embodiments, is configured to improve detection of a single analyte. For example, a fluidic medium, in some embodiments, is configured to alter a shape, morphology, or orientation of a single analyte (e.g., a denaturing fluid, a chaotrope, an ionic fluid, a magnetic fluid). In another example, a fluidic medium, in some embodiments, is configured to facilitate emission of light from a detectable label (e.g., altered pH for improved light emission). A fluidic medium, in some embodiments, comprises an aqueous or non-aqueous solvent. A fluidic medium, in some embodiments, comprises an ionic species (e.g., a monatomic ionic species, a polyatomic ionic species). A fluidic medium, in some embodiments, comprises a surfactant, a denaturant, and/or a chaotrope. In some configurations, a fluidic medium, in some embodiments, comprises a species that is configured to prevent photodegradation, such as dithiobutylamine, tris(2-carboxyethyl)phosphine, dithiothreitol, and/or 2-mercaptoethanol. A fluidic medium, in some embodiments, comprises a triplet quenching substance, such cyclooctatetraene. A fluidic medium, in some embodiments, comprises a deoxygenated liquid medium. Additional useful configurations of fluidic mediums are described in Patent Cooperation Treaty Application No. PCT/US2022/019831, which is herein incorporated by reference in its entirety.

### Methods

In an aspect, provided herein is a method of detecting one or more probe binding interactions on a single-analyte array, the method comprising the steps of: a) obtaining the single-analyte array comprising a first address that contains a first probe complex and a second address that comprises a second probe complex, in which the first probe complex comprises a first affinity reagent bound to a first single-analyte, in which the first affinity reagent is configured to produce a first detectable signal, in which the second probe complex comprises a second affinity reagent bound to a second single-analyte, in which the second affinity reagent is configured to produce a second detectable signal, in which the first detectable signal differs from the second detectable signal, and in which the first address is spatially separated from the second address at a distance that is resolvable at single-analyte resolution; b) contacting the first address with a first excitation source, thereby producing the first detectable signal, in which the first excitation source is not detectable at the second address; c) contacting the second address with a second excitation source, thereby producing the second detectable signal, in which the second excitation source is not detectable at the first address; and d) detecting the first signal and the second signal on a sensor, in which the sensor comprises a first channel that is configured to detect the first signal and a second channel that is configured to detect the second signal. A method, as set forth herein, in some embodiments, is adapted to any suitable optical detection system, including a confocal scanning microscope (e.g., a linescan confocal scanning microscope).

In another aspect, provided herein is a method of detecting one or more probe binding interactions on a single-analyte array, the method comprising the steps of: a) obtaining the single-analyte array comprising a first address and a second address, wherein the first address comprises a first signal source that emits a first signal and the second address comprises a second signal source that emits a second signal, in which the first address is physically separated from the second address by a separation distance; and b) detecting the first signal and the second signal on an optical system comprising a sensor, in which the sensor comprises a first channel that detects the first signal and a second channel that detects the second signal, in which the first channel is spatially separated and coplanar with the second channel.

In some cases, a single-analyte array comprises an ordered array. In particular cases, an ordered array comprises a patterned array, as set forth herein. A patterned array, in some embodiments, is formed by a lithographic method. In some cases, a patterned array comprises a plurality of addresses, in which the plurality of addresses comprises a first address and a second address. In some configurations of a patterned array, a plurality of addresses is arranged in a circular pattern, a rectangular pattern, a triangular pattern, a hexagonal pattern, or a radial pattern. In some configurations of a single-analyte array, an address of the plurality of addresses is coupled to a single-analyte. A patterned single-analyte array, in some embodiments, comprises a plurality of addresses with an average address spacing between any two adjacent addresses of the plurality of addresses. An average address spacing for the plurality of addresses, in some embodiments, has a coefficient of variation of no more than about 100%, 90%, 80%, 70%, 60%, 50%, 40%, 30%, 20%, 15%, 10%, 5%, 1%, or less than 1%.

In other configurations, a single-analyte array comprises an unpatterned array. An unpatterned array, in some embodiments, comprises a plurality of addresses, in which the plurality of addresses comprises the first address and the second address. In some configurations, an unpatterned array comprises no patterned, ordered, or otherwise defined single-analyte binding sites. In some configurations, a plurality of single-analytes is bound to an unpatterned array such that an average spacing between any two adjacent single-analytes of the plurality of single-analytes, in some embodiments, has a coefficient of variability of at least about 1%, 5%, 10%, 15%, 20%, 30%, 40%, 50%, 60%, 70%, 80%, 90%, 100%, or more than 100%. In other configurations, a plurality of single-analytes is bound to an unpatterned array such that an average spacing between any two adjacent single-analytes of the plurality of single-analytes, in some embodiments, has a coefficient of variability of no more than about 100%, 90%, 80%, 70%, 60%, 50%, 40%, 30%, 20%, 15%, 10%, 5%, 1%, or less than 1%.

A method, as set forth herein, in some embodiments, comprises identifying a first signal source at a first address and a second signal source at a second address, in which the first address and the second address are separated by a separation distance. In some cases, a separation distance between a first address comprising a first signal source and a second address comprising a second signal source is at least a distance for distinguishing the first address from the second address at single-analyte resolution. In some cases, a distance for distinguishing a first address comprising a first signal source from a second address comprising a second signal source at single-analyte resolution comprises a distance between a peak signal intensity of the first address and a peak signal intensity of the second address. For example, a peak signal intensity, in some embodiments, is identified by determining a pixel on a sensor containing a largest accumulated voltage, in which the pixel is amongst a plurality of pixels that are configured to detect a signal source at an address on an array. In particular cases, a peak signal intensity of a first address and a peak signal intensity of a second address each have a magnitude that is at least twice a magnitude of a minimum signal intensity between the first address and the second address. In other cases, a distance for distinguishing a first address comprising a first signal source from a second address comprising a second signal source at single-analyte resolution comprises a distance between a peak averaged signal intensity of the first address and a peak averaged signal intensity of the second address. For example, a method, in some embodiments, involves averaging accumulated pixel voltage from 3×3 clusters of pixels on an array to identify two or more clusters of pixels with local maximized average accumulated pixel voltage, in which a separation distance between two adjacent clusters is determined from a distance between two central pixels of the two adjacent clusters.

A method, as set forth herein, in some embodiments, comprises identifying a first signal source at a first address and a second signal source at a second address, in which the first address and the second address are separated by a separation distance. A separation distance between a first address comprising a first signal source and a second address comprising a second signal source, in some embodiments, is at least 10 nanometers (nm), 25 nm, 50 nm, 100 nm, 150 nm, 200 nm, 250 nm, 300 nm, 400 nm, 500 nm, 600 nm, 700 nm, 800 nm, 900 nm, 1 micron (μm), 2 μm, 3 μm, 4 μm, 5 μm, 10 μm, 50 μm, 100 μm, or more than 100 μm. Alternatively or additionally, a separation distance between a first address comprising a first signal source and a second address comprising a second signal source, in some embodiments, is no more than about 100 μm, 50 μm, 10 μm, 5 μm, 4 μm, 3 μm, 2 μm, 1 μm, 900 nm, 800 nm, 700 nm, 600 nm, 500 nm, 400 nm, 300 nm, 250 nm, 200 nm, 150 nm, 100 nm, 50 nm, 25 nm, 10 nm, or less than 10 nm.

In some cases, a separation distance comprises a distance between a first address and a second address, in which the first address and the second address are non-adjacent addresses of the single-analyte array. For example, an array of polypeptides, in some embodiments, is contacted with a plurality of detectable affinity agents that bind to a subset of the polypeptides, in which a first address containing a first detectable affinity agent and a second address containing a second detectable affinity agent are separated by a separation distance that is larger than a distance between adjacent polypeptides on the array. In other cases, a separation distance comprises a distance between a first address and a second address, in which the first address and the second address are adjacent addresses of the single-analyte array.

A method, as set forth herein, in some embodiments, comprises detecting a signal source on a sensor comprising a pixel array. In some cases, a pixel array comprises a rectangular pixel array, in which a total pixel quantity of the pixel array is determined as a product of a lengthwise number of pixels and a widthwise number of pixel. A rectangular pixel array, in some embodiments, has an aspect ratio that is calculated as the ratio of the lengthwise number of pixels to the widthwise number of pixels. A rectangular pixel array, in some embodiments, has an aspect ratio of at least about 1:1, 1.5:1, 2:1, 3:1, 4:1, 5:1, 10:1, 15:1, 20:1, 25:1, 50:1, 100:1, or more than 100:1. Alternatively or additionally, a rectangular pixel array, in some embodiments, has an aspect ratio of no more than about 100:1, 50:1, 25:1, 20:1, 15:1, 10:1, 5:1, 4:1, 3:1, 2:1, 1.5:1, 1:1, or less than 1:1.

A method, as set forth herein, in some embodiments, comprises detecting a first signal source on a first channel and a second signal source on a second channel of a sensor comprising the first channel and the second channel. In some cases, a first channel comprises a first rectangular pixel array and a second channel comprises a second rectangular pixel array, in which the first rectangular pixel array is oriented on the solid support parallel to the second rectangular pixel array. In particular cases, a first rectangular pixel array is spatially separated from a second rectangular pixel array by a channel separation distance. A channel separation distance, in some embodiments, is defined as a distance between a centerline or centerpoint of a first rectangular pixel array and a centerline or centerpoint of a second rectangular pixel array. Alternatively, a channel separation distance, in some embodiments, is defined as a distance between an exterior pixel or column of pixels of a first rectangular pixel array and a nearest pixel or column of pixels of a second rectangular pixel array. In some cases, a channel separation distance is related to a separation distance of a first address and a second address of an array by a magnification of an objective lens of an optical detection system. In some cases, a multi-channel sensor comprises a first solid support comprising a first channel and a second solid support comprising the second channel. In particular cases, a multi-channel sensor comprises a first solid support and a second solid support, in which the first solid support and the second solid support are arranged in a coplanar fashion. In other particular cases, a multi-channel sensor comprises a first solid support and a second solid support, in which the first solid support and the second solid support are not arranged in a coplanar fashion.

A method, as set forth herein, in some embodiments, comprises contacting a signal source with an excitant, in which the excitant causes the signal source to produce a signal. In some cases, a method comprises contacting a first signal source with a first excitant. In some cases, a method further comprises contacting a second signal source with a second excitant. In other cases, a signal source spontaneously produces a signal without an excitant. An excitant, in some embodiments, comprises an electromagnetic excitant, a chemical excitant, a thermal excitant, or a combination thereof. In some cases, an electromagnetic excitant comprises a photon (e.g., a photon with a wavelength that is sufficient to stimulate release of a photon from a fluorescent or luminescent signal source). A chemical excitant, in some embodiments, includes any suitable chemical substrate that produces a detectable signal in the presence of a signal source (e.g., a substrate for horseradish peroxidase). A thermal excitant, in some embodiments, includes the addition or removal of heat from an illumination target to stimulate a release of a signal from a signal source (e.g., a luminescent moiety).

A method, as set forth herein, in some embodiments, comprises the steps of: i) forming a spatially-separated electromagnetic beam, wherein the spatially separated electromagnetic beam comprising a first light beam and a second light beam; and contacting the spatially-separated electromagnetic beam with the single-analyte array, wherein the first light beam contacts a first signal source, and wherein the second light beam contacts a second signal source. In some cases, a first light beam and/or a second light beam, in some embodiments, comprise an excitant (e.g., a light beam comprising a wavelength of light that is sufficient to stimulate release of a photon from a fluorescent or luminescent signal source). In some cases, forming a spatially-separated electromagnetic beam comprises passing an electromagnetic beam through a beam-splitting optical device (e.g., a prismatic beam-splitter, a dichroic beam-splitter, etc.). In some cases, a first light beam comprises a first wavelength of light, wherein the second light beam comprises a second wavelength of light, and wherein the first wavelength of light differs from the second wavelength of light. In some cases, a first light beam comprises a first wavelength of light and a second light beam comprises a second wavelength of light, in which the first wavelength differs from the second wavelength by at least about 5 nanometers (nm), 10 nm, 15 nm, 20 nm, 25 nm, 30 nm, 40 nm, 50 nm, 100 nm, 125 nm, 150 nm, 200 nm, 250 nm, 300 nm, 400 nm, 500 nm, or more than 500 nm. Alternatively or additionally, a first light beam, in some embodiments, comprises a first wavelength of light and a second light beam, in some embodiments, comprises a second wavelength of light, in which the first wavelength differs from the second wavelength by no more than about 500 nm, 400 nm, 300 nm, 250 nm, 200 nm, 150 nm, 125 nm, 100 nm, 50 nm, 40 nm, 30 nm, 25 nm, 20 nm, 15 nm, 10 nm, 5 nm, or less than 5 nm. In some cases, a first light beam and a second light beam comprises the same wavelength of light. In other cases, a first light beam and a second light beam do not comprise the same wavelength of light. In some cases, a first light beam comprises a first range of wavelengths and a second light beam comprises a second range of wavelengths, in which the first range of wavelengths overlaps with the second range of wavelengths. In other cases, a first light beam, in some embodiments, comprises a first range of wavelengths and a second light beam, in some embodiments, comprises a second range of wavelengths, in which the first range of wavelengths does not overlap with the second range of wavelengths.

A method, as set forth herein, in some embodiments, comprises contacting an illumination target with a light beam, in which the light beam is formed or focused as a point of light on the illumination target. A method, as set forth herein, in some embodiments, further comprises rastering a point of light over an illumination target. A method, as set forth herein, in some embodiments, comprises contacting an illumination target with a light beam, in which the light beam is formed or focused as a line of light on the illumination target. A method, as set forth herein, in some embodiments, further comprises translating a line of light over an illumination target. For example, the line, in some embodiments, is translated in a direction that is orthogonal to the longest dimension of the line. A method, as set forth herein, in some embodiments, comprises contacting an illumination target with a first light beam and a second light beam, in which the first light beam and/or the second light beam is formed or focused as a line of light on the illumination target. The line formed by the first light beam and the line formed by the second light beam can be spatially-separated from each other by a separation distance. In some cases, a first light beam, in some embodiments, forms a first line of light and a second light beam, in some embodiments, forms a second line of light, in which the first line of light is parallel to the second line of light, and wherein an average distance between the first line of light and the second line of light is a separation distance. The separation distance can be measured in the dimension that is orthogonal to the longest dimensions of the two lines on the illumination target. In a particular case, a first channel of a multi-channel sensor is oriented parallel to a first line of light, and a second channel of the multi-channel sensor is oriented parallel to a second line of light.

A method, as set forth herein, in some embodiments, comprises detecting a first signal from a first signal source at a first address and a second signal from a second signal source at a second address. In some cases, detecting a first signal and a second signal on an optical detection system comprising a multi-channel sensor comprises one or more steps of: i) passing the first signal and the second signal through an objective of the optical detection system; ii) passing the first signal and the second signal through a focusing device (e.g., an autofocus device) of the optical detection system; and iii) contacting the first signal with the first channel of the sensor, and contacting the second signal with the second channel of the sensor. In some cases, passing a first signal and a second signal through a focusing device further comprises simultaneously focusing the first signal and the second signal on a multi-channel sensor.

In another aspect, provided herein is a method of detecting one or more probe binding interactions on a single-analyte array, the method comprising: a) translating the single-analyte array through an optical system in time-delay integration mode, wherein the single-analyte array comprises a first address and a second address, wherein the first address comprises a first optical signal source that emits a first optical signal and the second address comprises a second optical signal source that emits a second optical signal, wherein the first address is physically separated from the second address by a distance that is resolvable at single-analyte resolution; and b) detecting the first signal and the second signal on a sensor, wherein the first optical signal is detected on a first channel of the sensor and the second optical signal is detected on a second channel of the sensor, and wherein the first optical signal and the second optical signal do not require separate focus adjustment over the duration of a time-delay integration scan of the single-analyte array.

A method, as set forth herein, in some embodiments, occurs in an optical detection system that experiences a temporal or spatial variation in temperature during the method. In some cases, the optical detection system is non-isothermal. In particular cases, a non-isothermal optical detection system comprises one or more internal heat sources. For example, an optical detection system, in some embodiments, comprises a plurality of heat sources (e.g., microprocessors, pumps, motors, etc.) that produce heat within the optical detection system during implementation of a detection method. In other configurations, a non-isothermal optical detection system comprises an external heat source. For example, an optical detection system, in some embodiments, is situated in an environment (e.g., a laboratory) with a diurnal ambient temperature variation. In some cases, an optical detection system is spatially non-isothermal. In other cases, an optical detection system is temporally non-isothermal. In some cases, an optical detection system is both spatially and temporally non-isothermal. In other cases, an optical detection system is not spatially and/or temporally non-isothermal.

A method, as set forth herein, in some embodiments, detects a signal from a signal source in time-delay integration mode. A time-delay integration mode, in some embodiments, utilizes a method of detection in which an illumination target is translated relative to a sensor during detection. Data collected during a time-delay integration mode method, in some embodiments, includes temporal and spatial data for a signal from a signal source. In some cases, an optical detection system operates in a synchronous time delay integration mode (e.g., a pixel shift rate of a TDI sensor corresponds to a translation rate of a single-analyte array). In other cases, an optical detection system operates in an asynchronous time delay integration mode (e.g., a pixel shift rate of a TDI sensor does not correspond to a translation rate of a single-analyte array).

A method, as set forth herein, in some embodiments, comprises passing a signal from a signal source through a focusing device. A method, in some embodiments, further comprises a step of adjusting focus of an optical detection system utilizing a focusing device. Focus of an optical detection system, in some embodiments, is adjusted by altering a working distance between an objective lens and an illumination target (e.g., by translating the objective lens along an axis perpendicular to a surface of an illumination target). A method, as set forth herein, in some embodiments, comprises passing a first signal from a first signal source and a second signal from a second signal source through a focusing device. In some cases, a method further comprises one or more steps of: i) passing a first signal and a second signal through a focusing device; ii) detecting the first signal and the second signal on a sensor; iii) based upon the detected first signal and second signal, identifying a focus correction for the focusing device; and iv) after applying the focus correction to the focusing device, detecting the first signal and the second signal on the sensor. In some cases, a method further comprises a step of, after applying the focus correction to the focusing device, contacting a signal source with a light beam. For example, after correcting a focal point of an optical detection based upon one or more detected signals from a signal source, a signal source, in some embodiments, is again contacted with a light beam to produce another signal from the signal source.

In some cases, an optical detection system, as set forth herein, is used to detect presence or absence of a single-analyte, or to detect presence or absence of an interaction of a single-analyte with another reagent (e.g., binding of an affinity reagent to the single-analyte, gain or loss of a product formed by reaction of the single-analyte with a chemically reactive reagent, etc.). In other cases, an optical detection system, as set forth herein, is used to detect a property of a single-analyte (e.g., size, shape, conformation, sequence, identity, electrical charge, etc.).

In a particular method, an optical detection system, in some embodiments, is utilized to detect binding of a plurality of affinity agents to an array comprising a plurality of single-analytes (e.g., polypeptides, nucleic acids, etc.). In some cases, each address of an array of single-analytes comprises an unknown single-analyte. In some cases, each address of an array of single-analytes comprises no more than one single-analyte. In some cases, an optical detection system is utilized to detect binding of a plurality of affinity agents to an array comprising a plurality of single-analytes, in which an affinity agent of the plurality of affinity agents comprises a binding affinity for more than one single-analyte of the plurality of single-analytes. For example, a given affinity reagent, in some embodiments, is promiscuous with regard to binding to a variety of different analytes. In other cases, an optical detection system is utilized to detect binding of a plurality of affinity agents to an array comprising a plurality of single-analytes, in which an affinity agent of the plurality of affinity agents does not comprise a binding affinity for more than one single-analyte of the plurality of single-analytes. For example, a given affinity reagent, in some embodiments, is specific with regard to selectively binding to a particular analyte without appreciably binding to other analytes. In some cases, binding of an affinity agent to a single-analyte forms a detectable complex that produces a detectable signal (e.g., a fluorescent signal).

In a particular case, an optical detection system is utilized to determine an identity of a single-analyte at an address of a single-analyte array. A method of determining an identity of a single-analyte at an address of a single-analyte array, in some embodiments, comprises one or more steps of: a) contacting the single-analyte array comprising a plurality of addresses with a first plurality of affinity agents, in which each address of the plurality of addresses comprises a single-analyte, and in which each affinity agent of the first plurality of affinity agents comprises a detectable label; b) detecting a presence or absence of a signal from an affinity agent at each address of the single-analyte array with an optical detection system; c) repeating steps a) and b) with one or more additional plurality of affinity agents; and d) based upon the presence or absence of signal from each of the two or more affinity agents, determining an identity of the single-analyte at the address of the plurality of addresses. In some cases, a method of determining an identity of a single-analyte at an address of a single-analyte array includes one or more multiplexed steps. For example, a method, in some embodiments, comprises the steps of: a) contacting the single-analyte array comprising a plurality of addresses with a first plurality of affinity agents, in which each address of the plurality of addresses comprises a single-analyte, in which each affinity agent of the first plurality of affinity agents comprises a detectable label, in which the plurality of affinity agents comprises a first affinity agent comprising a first binding specificity for a first subset of single-analytes on the single-analyte array and a second affinity agent comprising a second binding specificity for a second subset of single-analytes on the single-analyte array, and in which the first affinity agent comprises a first detectable label that produces a first signal and the second affinity agent comprises a second detectable label that produces a second signal; b) detecting a presence or absence of the first signal and the second signal at each address on the single-analyte array with an optical detection system; and c) optionally repeating steps a) and b).

In another particular case, an optical detection system is utilized to determine a sequence of a sequenceable single-analyte (e.g., nucleic acids, polypeptides, etc.). In some cases, determining a sequence of a sequenceable single-analyte comprises a constructive sequencing assay (e.g., incorporating fluorescent nucleotides into a complementary strand of a nucleic acid by a polymerase extension reaction). In other cases, determining a sequence of a sequenceable single-analyte comprises a degradative sequencing assay (e.g., sequencing a polypeptide by an Edman-like degradation method). A degradative sequencing assay, in some embodiments, comprises one or more steps of: a) modifying a moiety (e.g., a nucleotide, an amino acid) of a single-analyte of a plurality of single-analytes on a single-analyte array, in which the single-analyte of the plurality of single-analytes is bound to an address of a plurality of addresses on the single-analyte array; b) contacting the single-analyte array with a plurality of affinity agents, in which an affinity agent of the plurality of affinity agents comprises a binding specificity for a modified moiety of the single-analyte, and in which each affinity agent comprises a detectable label that is configured to produce a signal; c) detecting a presence or absence of the signal from the affinity agent at each address of the plurality of addresses with an optical detection system; d) removing the modified moiety from the single-analyte; and e) optionally repeating any one of steps a)-d). Alternatively, a degradative sequencing assay, in some embodiments, comprises one or more steps of: a) modifying one or more moieties (e.g., nucleotides, amino acids) of a single-analyte of a plurality of single-analytes on a single-analyte array, in which the single-analyte of the plurality of single-analytes is bound to an address of a plurality of addresses on the single-analyte array; b) detecting a presence of one or more modified moieties of the single-analyte at the address with an optical detection system; c) removing a modified moiety of the one or more modified moieties from the single-analyte; d) detecting an absence of the modified moiety of the one or more modified moieties of the single-analyte at the address with an optical detection system; and e) optionally repeating any one of steps a)-d). A degradative sequencing assay, as set forth herein, in some embodiments, includes one or more multiplexed steps. For example, a multiplexed degradative sequencing assay, in some embodiments, utilizes a plurality of affinity agents, in which the plurality of affinity agents comprises a first affinity agent comprising a binding specificity for a first modified moiety, and a second affinity agent comprising a binding specificity for a second modified moiety, in which the first modified moiety and the second modified moiety differ, and in which the first affinity agent comprises a first detectable label that produces a first signal, and the second affinity agent comprises a second detectable label that produces a second signal, in which the first signal differs from the second signal. In another example, a degradative sequencing assay, in some embodiments, includes a step of co-labeling a subset of amino acids of a polypeptide (e.g., lysine, cysteine, tryptophan, tyrosine, etc.), in which each type of amino acid is provided a distinguishable detectable label (e.g., a first fluorophore unique to all lysines, a second fluorophore unique to all cysteines, etc.), then determining the quantity of each labeled amino acid in a polypeptide by measuring fluorescence intensity for each detectable label at an address containing the polypeptide with an optical detection system.

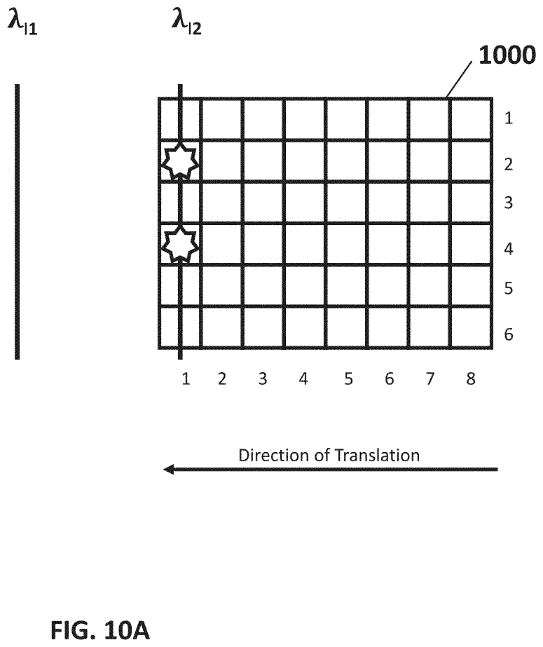

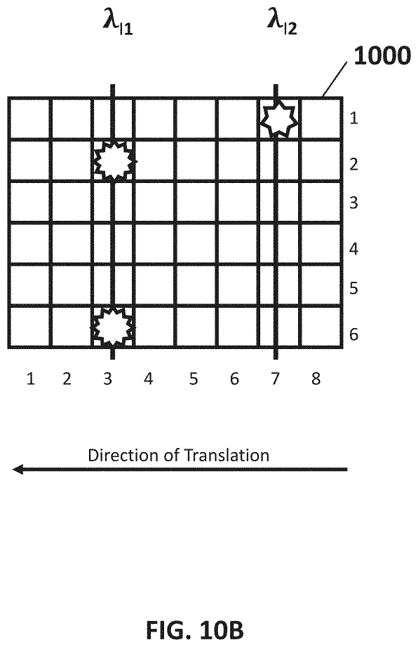

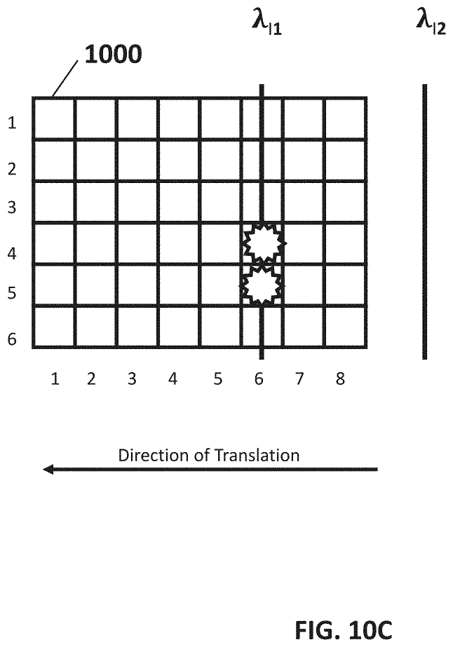

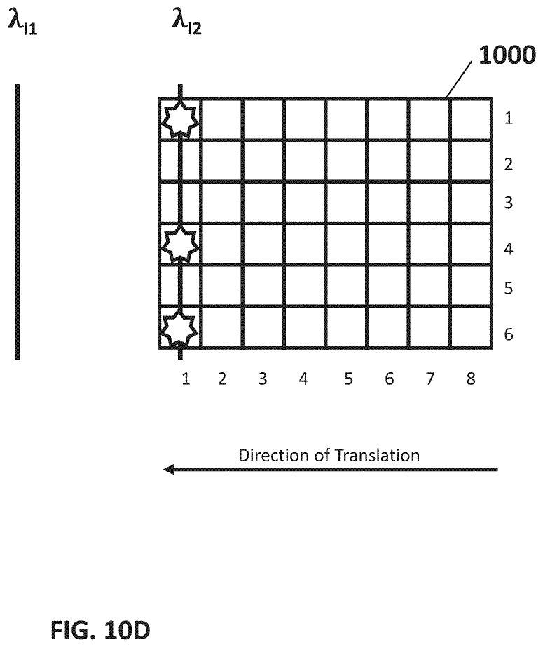

FIGS. 10A-10D illustrate multiplexed time-delay integration (TDI) optical detection for an array-based method (e.g., affinity binding measurements, fluorosequencing measurements, etc.) that, in some embodiments, is implemented for a single-analyte array. FIG. 10A depicts a single-analyte array 1000, comprising a single-analyte at each single-analyte binding site, at an initial time t₁, at which a line of light with wavelength λ_{I2} is contacting column 1 of the single-analyte array 1000. A channel of a multi-channel sensor that is configured to detect light of wavelength λ_{E2} (the emission wavelength for a signal of a detectable label stimulated by light of wavelength λ_{I2}) detects distinguishable signals at addresses (1, 2) and (1, 4) as defined by a (column, row) format. FIG. 10B depicts the single-analyte array at time t₂, at which the line of light with wavelength λ_{I2} is contacting addresses of the single-analyte array 1000 in column 7 and a line of light of wavelength is contacting addresses of the single-analyte array 1000 in column 3. The channel of the multi-channel sensor that is configured to detect light of wavelength λ_{E2} detects distinguishable signals at address (7, 1), and a channel of the multi-channel sensor that is configured to detect light of wavelength λ_{E1} (the emission wavelength for a signal of a detectable label stimulated by light of wavelength λ_{I1}) detects distinguishable signals at addresses (3, 2) and (3, 6). FIG. 10C depicts the single-analyte array at time t₃, at which the line of light with wavelength λ_{I2} is no longer contacting the single-analyte array 1000 and the line of light with wavelength λ_{I1} is contacting addresses of the single-analyte array 1000 in column 6. The channel of the multi-channel sensor that is configured to detect light of wavelength λ_{E1} detects a distinguishable signal at addresses (6, 4) and (6, 5). FIG. 10D depicts a start of a second cycle of multiplexed detection at a time t₄, at which the line of light with wavelength λ_{I2} is contacting column 1 of the single-analyte array 1000. The single-analyte array 1000 has been altered between the cycle depicted in FIGS. 10A-10C and the cycle depicted in FIG. 10D (e.g., binding of a second plurality of affinity agents with differing binding specificities, Edman-type degradation of single-analytes, etc.). The channel of the multi-channel sensor that is configured to detect light of wavelength λ_{E2} detects distinguishable signals at addresses (1, 1), (1, 4), and (1, 6). The pattern of optical signals detected by an optical detection system comprising the multi-channel sensor at each address of the single-analyte array 1000, in some embodiments, is utilized to determine a property or interaction for at least a subset of single-analytes at a subset of addresses of the single-analyte array 1000.

An optical detection system with a single, multi-channel sensor, in some embodiments, is advantageous for detection of low-frequency and/or low-probability phenomena, for example affinity agent binding to single-analytes in array-based formats. Such a system, in some embodiments, is configured to measure binding of an affinity agent that has a binding specificity for a small percentage of single-analytes on the array (e.g., no more than 0.1%, 0.5%, 1%, 5%, 10%, 20%, 25%, etc.). Additionally, each affinity agent, in some embodiments, has a probability of binding a single-analyte for which it has a binding specificity (e.g., no more than a 50%, 40%, 30%, 25%, 20%, 15%, 10%, 5%, 1%, 0.5%, 0.1% probability of binding a polypeptide to which it has a binding specificity). In the case of measuring binding of an affinity agent that is expected to bind 10% of single-analytes on an array of single-analytes, and a 25% probability of binding a single-analyte to which it has a specificity, the affinity agent is only expected to be observed at about 2.5% of total occupied binding sites on the array. For cases of low binding site occupancy, higher levels of stochastic error can exist in any measurements utilized to determine optimal focal depth for an image or set of images. For a multi-sensor optical detection system, a necessary computational load for optimizing focal position for each sensor, in some embodiments, is increased based upon the total number of sensors. In contrast, an optical detection system with a single, multi-channel sensor, in some embodiments, decreases a necessary computational load for optimizing focal position due to the simultaneous focusing of all detected light beams through a single emission or transmission pathway. Moreover, the simultaneous detection of multiple different signal sources on a single sensor, in some embodiments, increases the overall likelihood of receiving a signal from any array binding site, thereby decreasing the level of stochastic error of optical measurements utilized.

A method, as set forth herein, in some embodiments, comprises detecting two detectable labels that are co-localized at a same address on a single-analyte array, as set forth herein. In some cases, a first detectable label of a pair of co-localized detectable labels is coupled to a single analyte (e.g., directly-coupled to the single analyte, coupled to a linking moiety that is coupled to the analyte). In some cases, a second detectable label of a pair of co-localized detectable labels is coupled to a detectable probe that is coupled to a single analyte. In some particular cases, a detectable probe comprising a second detectable label is non-covalently coupled to a single analyte. In other particular cases, a detectable probe comprising a second detectable label is covalently coupled to a single analyte.

A method of detecting two detectable labels that are co-localized at a same address, in some embodiments, comprises the steps of: a) detecting presence or absence of a first detectable signal from a first detectable label at a plurality of addresses of a single-analyte array; b) detecting presence or absence of a second detectable signal from a second detectable label at the plurality of addresses of the single-analyte array; and c) identifying a presence of the first detectable signal and the second detectable signal at an address of the plurality of addresses. In some cases, the first detectable signal differs from the second detectable signal (e.g., a 488 nm first signal and a 647 nm second signal). Optionally, a method of detecting two detectable labels that are co-localized at a same address, in some embodiments, further comprises the steps of: d) removing the second detectable label from the single-analyte array, and e) after removing the second detectable label, detecting an absence of a second signal from the second detectable label at the address of the plurality of addresses. For example, during an affinity reagent-based polypeptide assay, a detectable affinity reagent, in some embodiments, is bound to single analytes, then subsequently removed, in which presence followed by absence of a detectable signal from the affinity reagent confirms a successful removal of the affinity reagent from the single analyte. Optionally, a method of detecting two detectable labels that are co-localized at a same address, in some embodiments, further comprises the step of identifying an address of a plurality of addresses comprising an absence of a first detectable signal from a first detectable label and a presence of a second detectable signal from a second detectable label. In some cases, an absence of a first detectable signal from a first detectable label and a presence of a second detectable signal from a second detectable label, in some embodiments, comprise a non-specific binding interaction (e.g., an unwanted or unexpected binding interaction) of the second detectable label with an address of a single-analyte array.

In some methods set forth herein, a detectable label or a detectable probe, in some embodiments, is non-covalently bound to an analyte. A method, in some embodiments, comprises a step of removing a non-covalently bound detectable label or probe from an analyte. Removing a non-covalently bound detectable label or probe from an analyte, in some embodiments, comprises dissociating the probe from the analyte. Methods of dissociation are known in the art and can include: i) providing a dissociation medium (e.g., a fluidic medium comprising a chaotrope, denaturant, or surfactant), ii) altering a fluidic medium (e.g., altering a pH, ionic strength, or chemical composition of a fluidic medium), iii) heating the probe and analyte, and iv) combinations thereof.

In some methods set forth herein, a detectable label or a detectable probe, in some embodiments, is covalently bound to an analyte. For example, peptide fluorosequencing, in some embodiments, utilize removal of fluorophore-labeled amino acids (and resultant loss or decrease in optical signal) to identify peptides. A method, in some embodiments, comprises a step of removing a covalently bound detectable label or probe from an analyte. Removing a covalently bound detectable label or probe from an analyte, in some embodiments, comprises chemical separation (e.g., an Edman-type degradation), enzymatic separation (e.g., via exonucleases, exopeptidases, etc.), or photo-induced cleavage (e.g., photon-mediated disruption of a photocleavable linker coupling a probe to an analyte).

### Polypeptide Assays

The present disclosure provides systems, compositions, and methods for forming particles that are useful for coupling single-analytes. The present disclosure further provides systems, compositions, and methods for forming single-analyte arrays that are useful when performing various single-analyte assays, including assays of biological analytes (e.g., genomics, transcriptomics, proteomics, metabolomics, etc.) and non-biological analytes (e.g., carbon nanoparticles, inorganic nanoparticles, etc.). In some configurations, the provided single-analyte arrays is especially useful for single-polypeptide proteomic assays such as, for example affinity reagent-based characterization assays (e.g., fluorescence-based or barcode-based affinity binding characterizations) or peptide sequencing assays (e.g., Edman-type degradation fluorosequencing or affinity reagent-based assays).

The present disclosure further provides methods for detecting one or more polypeptide (e.g. sample polypeptide, standard polypeptide etc.) or polypeptide product (e.g. sample polypeptide composite, standard polypeptide composite, etc.). A polypeptide can be detected using one or more probes having known binding affinity for the polypeptide. The probe and/or the polypeptide can be bound to form a complex and then formation of the complex can be detected. The complex can be detected directly, for example, due to a label that is present on the probe or polypeptide. In some configurations the complex need not be directly detected, for example, in formats where the complex is formed and then the probe, polypeptide, or a tag or label component that was present in the complex is then detected.

In some detection assays, a protein can be cyclically modified and the modified products from individual cycles can be detected. In some configurations, a protein can be sequenced by a sequential process in which each cycle includes steps of labeling and removing the amino terminal amino acid of a protein and detecting the label. Accordingly, a method of detecting a protein can include steps of (i) exposing a terminal amino acid on the protein; (ii) detecting a change in signal from the protein; and (iii) identifying the type of amino acid that was removed based on the change detected in step (ii). The terminal amino acid can be exposed, for example, by removal of one or more amino acids from the amino terminus or carboxyl terminus of the protein. Steps (i) through (iii) can be repeated to produce a series of signal changes that is indicative of the sequence for the protein.

In a first configuration of the above method, one or more types of amino acids in the protein can be attached to a label that uniquely identifies the type of amino acid. In this configuration, the change in signal that identifies the amino acid can be loss of signal from the respective label. Exemplary compositions and techniques that can be used to remove amino acids from a protein and detect signal changes are those set forth in Swaminathan et al., Nature Biotech. 36:1076-1082 (2018); or U.S. Pat. No. 9,625,469 or 10,545,153, each of which is incorporated herein by reference. Methods and apparatus under development by Erisyon, Inc. (Austin, Tex.), in some embodiments, are also useful for detecting proteins.

In a second configuration of the above method, the terminal amino acid of the protein can be recognized by an affinity agent that is specific for the terminal amino acid or specific for a label moiety that is present on the terminal amino acid. The affinity agent can be detected on the array, for example, due to a label on the affinity agent. Optionally, the label is a nucleic acid barcode sequence that is added to a primer nucleic acid upon formation of a complex. The formation of the complex and identity of the terminal amino acid can be determined by decoding the barcode sequence. Exemplary affinity agents and detection methods are set forth in US Pat. App. Pub. No. 2019/0145982 A1; 2020/0348308 A1; or 2020/0348307 A1, each of which is incorporated herein by reference. Methods and apparatus under development by Encodia, Inc. (San Diego, Calif.), in some embodiments, are also useful for detecting proteins.

Cyclical removal of terminal amino acids from a protein can be carried out using an Edman-type sequencing reaction in which a phenyl isothiocyanate reacts with a N-terminal amino group under mildly alkaline conditions (e.g. about pH 8) to form a cyclical phenylthiocarbamoyl Edman complex derivative. The phenyl isothiocyanate, in some embodiments, is substituted or unsubstituted with one or more functional groups, linker groups, or linker groups containing functional groups. An Edman-type sequencing reaction can include variations to reagents and conditions that yield a detectable removal of amino acids from a protein terminus, thereby facilitating determination of the amino acid sequence for a protein or portion thereof. For example, the phenyl group can be replaced with at least one aromatic, heteroaromatic or aliphatic group which, in some embodiments, participates in an Edman-type sequencing reaction, non-limiting examples including: pyridine, pyrimidine, pyrazine, pyridazoline, fused aromatic groups such as naphthalene and quinoline), methyl or other alkyl groups or alkyl group derivatives (e.g., alkenyl, alkynyl, cyclo-alkyl). Under certain conditions, for example, acidic conditions of about pH 2, derivatized terminal amino acids, in some embodiments, are cleaved, for example, as a thiazolinone derivative. The thiazolinone amino acid derivative under acidic conditions, in some embodiments, forms a more stable phenylthiohydantoin (PTH) or similar amino acid derivative which can be detected. This procedure can be repeated iteratively for residual protein to identify the subsequent N-terminal amino acid. Many variations of Edman-type degradation have been described and, in some embodiments, are used including, for example, a one-step removal of an N-terminal amino acid using alkaline conditions (Chang, J. Y., FEBS LETTS., 1978, 91(1), 63-68). In some cases, Edman-type reactions, in some embodiments, are thwarted by N-terminal modifications which, in some embodiments, are selectively removed, for example, N-terminal acetylation or formylation (e.g., see Gheorghe M. T., Bergman T. (1995) in Methods in Protein Structure Analysis, Chapter 8: Deacetylation and internal cleavage of Proteins for N-terminal Sequence Analysis. Springer, Boston, Mass. https://doi.org/ 10.1007/978-1-4899-1031-8_8).

Non-limiting examples of functional groups for substituted phenyl isothiocyanate, in some embodiments, include ligands (e.g. biotin and biotin analogs) for known receptors, labels such as luminophores, or reactive groups such as click functionalities (e.g. compositions having an azide or acetylene moiety). The functional group, in some embodiments, is a DNA, RNA, peptide or small molecule barcode or other tag which is further processed and/or detected.

The removal of an amino terminal amino acid using Edman-type processes utilizes at least two main steps, the first step includes reacting an isothiocyanate or equivalent with protein N-terminal residues to form a relatively stable Edman complex, for example, a phenylthiocarbamoyl complex. The second step includes removing the derivatized N-terminal amino acid, for example, via heating. The protein, now having been shortened by one amino acid, is detected in some embodiments, for example, by contacting the protein with a labeled affinity agent that is complementary to the amino terminus and examining the protein for binding to the agent, or by detecting loss of a label that was attached to the removed amino acid.

Edman-type processes can be carried out in a multiplex format to detect, characterize or identify a plurality of proteins. A method of detecting a protein can include steps of (i) exposing a terminal amino acid on a protein at an address of an array; (ii) binding an affinity agent to the terminal amino acid, where the affinity agent comprises a nucleic acid tag, and where a primer nucleic acid is present at the address; (iii) extending the primer nucleic acid, thereby producing an extended primer having a copy of the tag; and (iv) detecting the tag of the extended primer. The terminal amino acid can be exposed, for example, by removal of one or more amino acids from the amino terminus or carboxyl terminus of the protein. Steps (i) through (iv) can be repeated to produce a series of tags that is indicative of the sequence for the protein. The method can be applied to a plurality of proteins on the array and in parallel. Whatever the plexity, the extending of the primer can be carried out, for example, by polymerase-based extension of the primer, using the nucleic acid tag as a template. Alternatively, the extending of the primer can be carried out, for example, by ligase- or chemical-based ligation of the primer to a nucleic acid that is hybridized to the nucleic acid tag. The nucleic acid tag can be detected via hybridization to nucleic acid probes (e.g. in an array), amplification-based detections (e.g. PCR-based detection, or rolling circle amplification-based detection) or nuclei acid sequencing (e.g. cyclical reversible terminator methods, nanopore methods, or single molecule, real time detection methods). Exemplary methods that can be used for detecting proteins using nucleic acid tags are set forth in US Pat. App. Pub. No. 2019/0145982 A1; 2020/0348308 A1; or 2020/0348307 A1, each of which is incorporated herein by reference.

Polypeptides can also be detected based on their enzymatic or other biological activity. For example, a polypeptide can be contacted with a reactant that is converted to a detectable product by an enzymatic activity of the polypeptide. In other assay formats, a first polypeptide having a known enzymatic function can be contacted with a second polypeptide to determine if the second polypeptide changes the enzymatic function of the first polypeptide. As such, the first polypeptide serves as a reporter system for detection of the second polypeptide. Exemplary changes that can be observed include, but are not limited to, activation of the enzymatic function, inhibition of the enzymatic function, degradation of the first polypeptide or competition for a reactant or cofactor used by the first polypeptide.

The presence or absence of post-translational modifications (PTM) can be detected using a composition, apparatus or method set forth herein. A PTM can be detected using an affinity agent that recognizes the PTM or based on a chemical property of the PTM. Exemplary PTMs that can be detected, identified or characterized include, but are not limited to, myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglyclyation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, proteolytic cleavage, isoaspartate formation, racemization, and protein splicing.

PTMs, in some embodiments, occur at particular amino acid residues of a protein. For example, the phosphate moiety of a particular proteoform can be present on a serine, threonine, tyrosine, histidine, cysteine, lysine, aspartate or glutamate residue of the protein. In other examples, an acetyl moiety can be present on the N-terminus or on a lysine; a serine or threonine residue can have an O-linked glycosyl moiety; an asparagine residue can have an N-linked glycosyl moiety; a proline, lysine, asparagine, aspartate or histidine amino acid can be hydroxylated; an arginine or lysine residue can be methylated; or the N-terminal methionine or at a lysine amino acid can be ubiquitinated.

Polypeptides can also be detected based on their binding interactions with other molecules such as polypeptides (e.g. with or without post translational modifications), nucleic acids, nucleotides, metabolites, small molecules that participate in biological signal transduction pathways, biological receptors or the like. For example, a polypeptide that participates in a signal transduction pathway can be identified by detecting binding of the polypeptide with a second polypeptide that is known to be its binding partner in the pathway. Generally, a target polypeptide can be conjugated to a SNAP or SNAP complex and then contacted with a probe polypeptide, or other probe molecule, that is known to have affinity for the polypeptide. The target polypeptide can be identified based on observed binding by the probe molecule or lack of binding by the probe molecule. The probe molecule can optionally be labeled using labels set forth herein or known in the art.

In some configurations of the polypeptide detection methods set forth herein, the polypeptides can be detected on a solid support. For example, polypeptides can be attached to a support, the support can be contacted with probes in solution, the probes can interact with the polypeptides, thereby producing a detectable signal, and then the signal can be detected to determine the presence of the polypeptides. In multiplexed versions of this approach, different polypeptides can be attached to different addresses in an array, and the probing and detection steps can occur in parallel. In another example, probes can be attached to a solid support, the support can be contacted with polypeptides in solution, the polypeptides can interact with the probes, thereby producing a detectable signal, and then the signal can be detected to determine the presence of the polypeptides. This approach can also be multiplexed by attaching different probes to different addresses of an array. Polypeptides can be attached to a support via conjugation to SNAPs or SNAP complexes. For example, a plurality of polypeptides can be conjugated to a plurality of SNAPs or SNAP complexes, such that each polypeptide-conjugated SNAP or SNAP complex forms an address in the array. In yet another approach, polypeptides can be detected using mass spectrometry methods. Several exemplary detection methods are set forth below and elsewhere herein. It will be understood that other detection methods can also be used.

Typical polypeptide detection methods, such as enzyme linked immunosorbent assay (ELISA), achieve high-confidence characterization of one or more polypeptide in a sample by exploiting high specificity binding of antibodies, aptamers or other binding reagents to the polypeptide(s) and detecting the binding event while ignoring all other polypeptides in the sample. ELISA is generally carried out at low plex scale (e.g. from one to several hundred different polypeptides detected in parallel or in succession) but can be used at higher plexity. One or more polypeptides can be conjugated to one or more SNAPs or SNAP complexes and the conjugated polypeptide(s) can be detected using ELISA.

ELISA methods can be carried out by detecting immobilized binding reagents and/or polypeptides in multiwell plates, detecting immobilized binding reagents and/or polypeptides on arrays, or detecting immobilized binding reagents and/or polypeptides on particles in microfluidic devices. Exemplary plate-based methods include, for example, the MULTI-ARRAY technology commercialized by MesoScale Diagnostics (Rockville, Md.) or Simple Plex technology commercialized by Protein Simple (San Jose, Calif.). Exemplary, array-based methods include, but are not limited to those utilizing Simoa® Planar Array Technology or Simoa® Bead Technology, commercialized by Quanterix (Billerica, Mass.). Further exemplary array-based methods are set forth in U.S. Pat. Nos. 9,678,068; 9,395,359; 8,415,171; 8,236,574; or 8,222,047, each of which is incorporated herein by reference. Exemplary microfluidic detection methods include those commercialized by Luminex (Austin, Tex.) under the trade name xMAP® technology or used on platforms identified as MAGPIX®, LUMINEX® 100/200 or FEXMAP 3D®. Plate-based methods of microfluidic detection methods can be modified to use SNAPs or SNAP complexes as set forth herein.

Other detection methods that can also be used, and that are particularly useful at low plex scale include procedures that employ SOMAmer reagents and SOMAscan assays commercialized by Soma Logic (Boulder, Colo.). In one configuration, a sample is contacted with aptamers that are capable of binding polypeptides with high specificity for the amino acid sequence of the polypeptides. The resulting aptamer-polypeptide complexes can be separated from other sample components, for example, by attaching the complexes to beads, SNAPs or SNAP complexes that are removed from the sample. The aptamers can then be isolated and, because the aptamers are nucleic acids, the aptamers can be detected using any of a variety of methods known in the art for detecting nucleic acids, including for example, hybridization to nucleic acid arrays, PCR-based detection, or nucleic acid sequencing. Exemplary methods and compositions for use in an aptamer-based or other detection method set forth herein are set forth in U.S. Pat. Nos. 8,404,830; 8,975,388; 9,163,056; 9,938,314; 10,239,908; 10,316,321 or 10,221,207. Further examples are set forth in U.S. Pat. Nos. 7,855,054; 7,964,356; 8,975,026; 8,945,830; 9,404,919; 9,926,566; 10,221,421; 10,316,321 or 10,392,621. The above patents are incorporated herein by reference. The aptamers or polypeptides set forth above or in the above references can be attached to SNAPs or SNAP complexes as set forth herein.

Polypeptides can also be detected based on proximity of two or more probes. For example, two probes can each include a receptor component and a nucleic acid component. When the probes bind in proximity to each other, for example, due to ligands for the respective receptors being on a single polypeptide, or due to the ligands being present on two polypeptides that associate with each other, the nucleic acids can interact to cause a modification that is indicative of the proximity. For example, one of the nucleic acids can be extended using the other nucleic acid as a template, one of the nucleic acids can form a template that positions the other nucleic acid for ligation to another nucleic acid, or the like. Exemplary methods are commercialized by Olink Proteomics AB (Uppsala Sweden) or set forth in U.S. Pat. Nos. 7,306,904; 7,351,528; 8,013,134; 8,268,554 or 9,777,315, each of which is incorporated herein by reference. The polypeptides, probes, ligands or receptors set forth above or in the above references can be attached to SNAPs or SNAP complexes as set forth herein.

A method of detecting a polypeptide, can include a step of detecting a sample polypeptide (e.g. a sample polypeptide conjugate) and/or detecting a standard polypeptide (e.g. a standard polypeptide conjugate). In one configuration, detection can include steps of (i) contacting a first set of binding reagents with a sample polypeptide, and/or a standard polypeptide, and (ii) detecting binding of the sample polypeptide and/or standard polypeptide to a binding reagent in the second set of binding reagents. The method can optionally include one or more of the further steps of (iii) removing the first set of binding reagents, (iv) binding a second set of binding reagents to the sample polypeptide, and/or the standard polypeptide, where binding reagents in the second set are different from binding reagents in the first set, and (v) detecting binding of the sample polypeptide and/or standard polypeptide to a binding reagent in the second set of binding reagents. The method can optionally be carried out for one or more sample polypeptides in an array or standard polypeptides. Methods and apparatus that employ standard polypeptides are set forth in U.S. Pat. App. Ser. No. 63/139,818, which is incorporated herein by reference. The sample polypeptides or standard polypeptides set forth above or in the above reference can be attached to SNAPs or SNAP complexes as set forth herein.

High specificity binding reagents can be useful in a number of polypeptide detection methods. Alternatively, detection can be based on multiple low specificity detection cycles that are performed on a sample such that the individual cycles, in some embodiments, detect multiple polypeptides while not necessarily distinguishing one of the detected polypeptides from another in any one of the cycles. However, using compositions and methods set forth herein, results from multiple cycles can be combined to achieve high-confidence quantification, identification or characterizations of a plurality of individual polypeptides in the sample. In many embodiments, one or more of the individual cycles yield ambiguous results with regard to distinguishing the identity of a subset of polypeptides that produce detectable signal; however, characterizing the signals across the multiple cycles allows individual polypeptides to be individually and unambiguously identified. The resulting set of identified polypeptides can be larger than the number of polypeptides that produce signal from any of the individual cycles.

Some configurations of detection methods that are based on multiple low specificity detection cycles, in some embodiments, are understood, to some extent, via analogies to the children's game “20 Questions.” An objective of this game is to identify a target answer in as few questions as possible. An effective tactic is to ask questions on characteristics ranging from broad characteristics (e.g., “Is it a person, place, or thing?”, “Is the person in this room?”) to narrow characteristics (e.g., “Is the person named ‘Mary’?”). In general, it is possible to identify a character in the game by asking substantially fewer questions (N) than the possible number of answers (M), i.e. N<<M. By analogy, affinity reagents used in some configurations of the detection methods set forth herein, in some embodiments, have a broad range of interactions with respect to a population of polypeptides. For example, an affinity reagent, in some embodiments, is considered to be a ‘promiscuous’ affinity reagent due to its affinity for a single epitope that is present in a plurality of different polypeptides in a sample, or due to its affinity for a plurality of different epitopes that are present in one or more polypeptides in the sample. By testing for the interaction of an affinity reagent with a polypeptide, information is acquired regardless of whether an interaction is observed. For example, a failure of an affinity reagent to bind a polypeptide is indicative of the polypeptide lacking the epitope for the affinity reagent.

In the above-described analogy of 20 Questions, the outcome is based upon clear articulation of queries and answers, and is also based upon accurate and reliable answers (e.g., type, size, attributes, etc.). By analogy, polypeptide characterization by the measurement of affinity reagent interactions, in some embodiments, is more difficult when the measurements are prone to a degree of systematic or random error or uncertainty. For example, measurement accuracy of affinity reagent (e.g., antibody) interactions with binding targets (e.g. epitopes), in some embodiments, is affected by numerous factors such as system detection limits or sensitivity, non-specific interactions between epitopes and affinity reagents (false positives), or stochastic, time-dependent reversal of an interaction (false negatives).

It is not uncommon for polypeptide characterization measurements to contain a degree of uncertainty. High-confidence characterization, in some embodiments, is achieved by utilizing multiple low specificity detection cycles in combination with a probabilistic decoding approach. The overlaying or combining of binary polypeptide interaction data (e.g., affinity reagent A1, which interacts with epitope X, was not observed to interact with unknown polypeptide P, therefore, polypeptide P does not contain epitope X), in some embodiments, leads to improper polypeptide characterization due to the inclusion or exclusion of possible candidate states due to measurement error. By contrast, overlaying or combining probabilistic polypeptide interaction data, in some embodiments, permits an algorithm to converge to a high-confidence prediction of polypeptide identity without needing to exclude any candidate states. For example, if affinity reagents A1 to A6 are known to interact with a known polypeptide P1 with interaction probabilities, and measurable interactions of affinity reagents A2, A5 and A6 are observed against an unknown polypeptide P, in some embodiments it is concluded that polypeptide P is likely not polypeptide P1 (2 of 3 likely interactions were not observed; 2 of 3 unlikely interactions were observed). Moreover, a probability-based characterization, in some embodiments, is assigned a degree of confidence such that a prediction for each observed polypeptide is made when the degree of confidence rises above a threshold degree of confidence. For example, in the above observation of polypeptide P, the six described observations may not provide a high enough degree of confidence to eliminate polypeptide P1 as a possible identity, but similar trends over 20 or more affinity reagents may provide sufficient degree of confidence to eliminate P1 as a possible identity. Accordingly, polypeptide P1 can be subjected to binding reactions with a series of promiscuous affinity reagents, and although the observation from each binding reaction taken individually may be ambiguous with regard to identifying the polypeptide, decoding the observations from the series of binding reactions may identify polypeptide P1 with an acceptable level of confidence.

A polypeptide detection assay that is based on multiple low specificity detection cycles, in some embodiments, is configured to permit polypeptide characterization at an individual or single-molecule level. Polypeptides to be characterized, in some embodiments, are provided on a solid support containing unique, detectably resolvable characterization sites. For example, the polypeptides can be attached to the sites via conjugation to SNAPs or SNAP complexes. Such characterization sites, in some embodiments, are spaced, arrayed, or otherwise ordered to allow individual sites to be distinguished one from another when detecting their interactions with affinity reagents. A solid support, in some embodiments, comprises a sufficient number of unique, optically resolvable characterization sites to accommodate a plurality, majority, or all polypeptides from a sample, such as at least about 1×10⁴, 1×10⁵, 1×10⁶, 1×10⁷, 1×10⁸, 1×10⁹, 1×10¹⁰, 1×10¹¹, 1×10¹², or more than 1×10¹² sites. Each site, in some embodiments, contains a known number of polypeptides that are to be characterized. In some cases, a characterization site contains a single polypeptide molecule to be detected, identified or characterized. In other cases, a site contains multiple polypeptide molecules, with at least one molecule to be detected. For example, the polypeptide molecule to be detected can be one subunit in a larger protein having multiple different subunits.

In some cases, polypeptide detection assays that are based on multiple low specificity detection cycles utilizes affinity reagents such as antibodies (or functional fragments thereof), aptamers, mini protein binders, or any other suitable binding reagent. Affinity reagents, in some embodiments, are promiscuous affinity reagents that possess a likelihood to interact with (e.g., bind to) more than one polypeptide in a sample. In some cases, the affinity reagents possess a likelihood to interact with two or more unique, structurally dissimilar proteins in a sample. For example, an affinity reagent, in some embodiments, binds with near-equal probability to a particular membrane protein and a particular cytoplasmic protein based upon a region of structural similarity. In some cases, a binding affinity reagent possesses a likelihood of binding to a particular amino acid epitope or family of epitopes regardless of the sequence context (e.g., amino acid sequence upchain and/or downchain from the epitope). An affinity reagent can bind to a polypeptide that is conjugated to a SNAP or SNAP complex.

An affinity reagent that is used for multiple low specificity detection cycles, in some embodiments, is characterized such that it has an identified, determined, or assessed probability-based binding profile. An affinity reagent, in some embodiments, has the property of binding to a first polypeptide with an identified, determined, or assessed binding probability of greater than about 50% (e.g., at least about 50%, 60%, 70%, 80%, 90%, 95%, 99%, 99.9%, 99.99%, 99.999% or greater than about 99.999%) and binding to a second structurally non-identical polypeptide with an identified, determined, or assessed binding probability of less than about 50% (e.g., no more than about 50%, 40%, 30%, 20%, 10%, 5%, 1%, 0.1%, 0.01%, 0.001% or less than about 0.001%). In a particular case, the difference in observed binding probabilities of the affinity reagent to the first and second polypeptides is due to the presence, absence, or inaccessibility of a particular epitope or family of epitopes in either the first or second polypeptide. Probabilistic affinity reagent binding profiles, in some embodiments, are determined or identified by in vitro measurements or in silico predictions.

Polypeptide detection methods that are based on multiple low specificity detection cycles, in some embodiments, further incorporate computational decoding approaches that are optimized for the above-described affinity reagents. The decoding approaches, in some embodiments, overlay or combine data from multiple rounds of detecting affinity reagent interaction with individual polypeptides, and can assign a degree of confidence for detection of signal from each polypeptide. For example, affinity reagent interactions can be detected for each site in an array of sites, and a degree of confidence can be assigned to detection of each signal at each site. Similarly, a degree of confidence can be assigned to a series of detection events at each site. A polypeptide, in some embodiments, is considered identified or characterized if the degree of confidence for a prediction based upon overlayed or combined affinity reagent interaction data exceeds a threshold degree of confidence. The threshold degree of confidence for a polypeptide characterization prediction, in some embodiments, depends upon the nature of the characterization. The threshold degree of confidence, in some embodiments, falls in a range from about 50% to about 99.999%, such as about 50%, 60%, 70%, 80%, 90%, 95%, 99%, 99.99%, or about 99.999%. In some cases, the threshold degree of confidence is outside this range. In some cases, the computational decoding approaches incorporate machine learning or training algorithms to update or refine the determined or identified probabilistic interaction profile for the affinity reagents or polypeptides with increased information or in ever widening contexts.

Particularly useful methods and algorithms that can be used for detection methods employing multiple low specificity detection cycles are set forth, for example, in U.S. Pat. No. 10,473,654; or PCT Publication No. WO 2019/236749 A2; or US Pat. App. Pub. Nos. 2020/0082914 A1 or 2020/0090785 A1, each of which is incorporated herein by reference. The methods set forth above and in the preceding references can be modified to use SNAPs or SNAP complexes of the present disclosure, for example, to attach polypeptides to a solid support.

A method of detecting a polypeptide, can include a process of detecting a sample polypeptide, the process including steps of (i) binding a first binding reagent to a sample polypeptide at an address of an array, where the binding reagent comprises a nucleic acid tag, and where a primer nucleic acid is present at the address; (ii) extending the primer nucleic acid, thereby producing an extended primer having a copy of the tag; and (iii) detecting the tag of the extended primer. The polypeptide can be attached at the address of the array via conjugation to a SNAP or SNAP complex. The extending of the primer can be carried out, for example, by polymerase-based extension of the primer, using the nucleic acid tag as a template. Alternatively, the extending of the primer can be carried out, for example, by ligase or chemical based ligation of the primer to a nucleic acid that is hybridized to the nucleic acid tag. The nucleic acid tag can be detected via hybridization to nucleic acid probes (e.g. in a microarray), amplification-based detections (e.g. PCR-based detection, or rolling circle amplification-based detection) or nucleic acid sequencing (e.g. cyclical reversible terminator methods, nanopore methods, or single molecule, real time detection methods). Exemplary methods that can be used for detecting polypeptides using nucleic acid tags are set forth in US Pat. App. Pub. No. 2019/0145982 A1; 2020/0348308 A1; or 2020/0348307 A1, each of which is incorporated herein by reference.

A method of detecting a polypeptide, can include a process of detecting a sample polypeptide, the process including steps of (i) exposing a terminal amino acid on the polypeptide; (ii) detecting a change in signal from the polypeptide; and (iii) identifying the type of amino acid that was removed based on the change detected in step (ii). The terminal amino acid can be exposed, for example, by removal of one or more amino acids from the amino terminus or carboxyl terminus of the polypeptide. Steps (i) through (iii) can be repeated to produce a series of signal changes that is indicative of the sequence for the polypeptide. Optionally, one or more different polypeptides can be attached at respective addresses of a polypeptide array, for example, via conjugation to a SNAP or SNAP complex at the addresses. The signal change can optionally be detected at one or more address on an array.

In a first configuration of the above method, one or more types of amino acids in the polypeptide can be attached to a label that uniquely identifies the type of amino acid. In this configuration, the change in signal that identifies the amino acid can be loss of signal from the respective label. Exemplary compositions and techniques that can be used to remove amino acids from a polypeptide and detect signal changes are set forth in Swaminathan et al., Nature Biotech. 36:1076-1082 (2018); or U.S. Pat. No. 9,625,469 or 10,545,153, each of which is incorporated herein by reference. The polypeptide can be attached to a solid support via conjugation to a SNAP or SNAP complex.

In a second configuration of the above method, the terminal amino acid of the polypeptide can be recognized by a binding reagent that is specific for the terminal amino acid or specific for a label moiety that is present on the terminal amino acid. The binding reagent can be detected on the array, for example, due to a label on the binding reagent. Exemplary binding reagents and detection methods are set forth in US Pat. App. Pub. No. 2019/0145982 A1; 2020/0348308 A1; or 2020/0348307 A1, each of which is incorporated herein by reference. The polypeptide can be attached to a solid support via conjugation to a SNAP or SNAP complex.

A method of detecting a polypeptide can include a process of detecting a sample polypeptide of an array of polypeptides, the process including steps of (i) exposing a terminal amino acid on a polypeptide at an address of an array; (ii) binding a binding reagent to the terminal amino acid, where the binding reagent comprises a nucleic acid tag, and where a primer nucleic acid is present at the address; (iii) extending the primer nucleic acid, thereby producing an extended primer having a copy of the tag; and (iv) detecting the tag of the extended primer. The terminal amino acid can be exposed, for example, by removal of one or more amino acids from the amino terminus or carboxyl terminus of the polypeptide. Steps (i) through (iv) can be repeated to produce a series of tags that is indicative of the sequence for the polypeptide. The extending of the primer can be carried out, for example, by polymerase-based extension of the primer, using the nucleic acid tag as a template. Alternatively, the extending of the primer can be carried out, for example, by ligase- or chemical-based ligation of the primer to a nucleic acid that is hybridized to the nucleic acid tag. The nucleic acid tag can be detected via hybridization to nucleic acid probes (e.g. in a microarray), amplification-based detections (e.g. PCR-based detection, or rolling circle amplification-based detection) or nuclei acid sequencing (e.g. cyclical reversible terminator methods, nanopore methods, or single molecule, real time detection methods). Exemplary methods that can be used for detecting polypeptides using nucleic acid tags are set forth in US Pat. App. Pub. No. 2019/0145982 A1; 2020/0348308 A1; or 2020/0348307 A1, each of which is incorporated herein by reference. A polypeptide, primer nucleic acid or template nucleic acid copied by extension of the primer can be attached to a SNAP or SNAP complex.

A method of detecting can include determining a detected property such as a polypeptide sequence, presence of a known epitope, polypeptide size, polypeptide isoelectric point, polypeptide hydrophobicity, polypeptide hydrodynamic radius, polypeptide pKa, the presence of a post-translational modification, the absence of a post-translational modification, polypeptide charge, the presence of a non-natural amino acid or other non-natural amino acid chemical unit, the presence of secondary, tertiary, or quaternary structure, the absence of secondary, tertiary, or quaternary structure, presence of a bound molecule, or absence of a bound molecule. A bound non-polypeptide molecule, in some embodiments, comprises a chelated ion, a bound metal cluster, a bound cofactor (e.g., a porphyrin), a bound ligand, a bound substrate, or a bound biomolecule (e.g., polysaccharide, nucleic acid, protein, etc.).

A method or apparatus of the present disclosure can optionally be configured for optical detection (e.g. luminescence detection). Analytes or other entities can be detected, and optionally distinguished from each other, based on measurable characteristics such as the wavelength of radiation that excites a luminophore, the wavelength of radiation emitted by a luminophore, the intensity of radiation emitted by a luminophore (e.g. at particular detection wavelength(s)), luminescence lifetime (e.g. the time that a luminophore remains in an excited state) or luminescence polarity. Other optical characteristics that can be detected, and optionally used to distinguish analytes, include, for example, absorbance of radiation, resonance Raman, radiation scattering, or the like. A luminophore can be an intrinsic moiety of a protein or other analyte to be detected, or the luminophore can be an exogenous moiety that has been synthetically added to a protein or other analyte.

A method or apparatus of the present disclosure can use a light sensing device that is appropriate for detecting a characteristic set forth herein or known in the art. Particularly useful components of a light sensing device can include, but are not limited to, optical sub-systems or components used in nucleic acid sequencing systems. Examples of useful sub systems and components thereof are set forth in US Pat. App. Pub. No. 2010/0111768 A1 or U.S. Pat. Nos. 7,329,860; 8,951,781 or 9,193,996, each of which is incorporated herein by reference. Other useful light sensing devices and components thereof are described in U.S. Pat. Nos. 5,888,737; 6,175,002; 5,695,934; 6,140,489; or 5,863,722; or US Pat. Pub. Nos. 2007/007991 A1, 2009/0247414 A1, or 2010/0111768; or WO2007/123744, each of which is incorporated herein by reference. Light sensing devices and components that can be used to detect luminophores based on luminescence lifetime are described, for example, in U.S. Pat. Nos. 9,678,012; 9,921,157; 10,605,730; 10,712,274; 10,775,305; or 10,895,534 each of which is incorporated herein by reference.

Luminescence lifetime can be detected using an integrated circuit having a photodetection region configured to receive incident photons and produce a plurality of charge carriers in response to the incident photons. The integrated circuit can include at least one charge carrier storage region and a charge carrier segregation structure configured to selectively direct charge carriers of the plurality of charge carriers directly into the charge carrier storage region based upon times at which the charge carriers are produced. See, for example, U.S. Pat. Nos. 9,606,058, 10,775,305, and 10,845,308, each of which is incorporated herein by reference. Optical sources that produce short optical pulses can be used for luminescence lifetime measurements. For example, a light source, such as a semiconductor laser or LED, can be driven with a bipolar waveform to generate optical pulses with FWHM durations as short as approximately 85 ps having suppressed tail emission. See, for example, in U.S. Pat. No. 10,605,730, which is incorporated herein by reference.

For configurations that use optical detection (e.g. luminescent detection), one or more analytes (e.g. proteins), in some embodiments, are immobilized on a surface, and this surface is scanned with a microscope to detect any signal from the immobilized analytes. The microscope itself, in some embodiments, comprises a digital camera or other luminescence detector configured to record, store, and analyze the data collected during the scan. A luminescence detector of the present disclosure can be configured for epiluminescent detection, total internal reflection (TIR) detection, waveguide assisted excitation, or the like.

A light sensing device, in some embodiments, is based upon any suitable technology, and, in some embodiments, is, for example, a charged coupled device (CCD) sensor that generates pixilated image data based upon photons impacting locations in the device. It will be understood that any of a variety of other light sensing devices, in some embodiments, are also used including, but not limited to, a detector array configured for time delay integration (TDI) operation, a complementary metal oxide semiconductor (CMOS) detector, an avalanche photodiode (APD) detector, a Geiger-mode photon counter, a photomultiplier tube (PMT), charge injection device (CID) sensors, JOT image sensor (Quanta), or any other suitable detector. Light sensing devices can optionally be coupled with one or more excitation sources, for example, lasers, light emitting diodes (LEDs), arc lamps or other energy sources known in the art.

An optical detection system can be configured for single molecule detection. For example, waveguides or optical confinements can be used to deliver excitation radiation to locations of a solid support where analytes are located. Zero-mode waveguides can be particularly useful, examples of which are set forth in U.S. Pat. Nos. 7,181,122, 7,302,146, or 7,313,308, each of which is incorporated herein by reference. Analytes can be confined to surface features, for example, to facilitate single molecule resolution. For example, analytes can be distributed into wells having nanometer dimensions such as those set forth in U.S. Pat. No. 7,122,482 or 8,765,359, or US Pat. App. Pub. No 2013/0116153 A1, each of which is incorporated herein by reference. The wells can be configured for selective excitation, for example, as set forth in U.S. Pat. No. 8,798,414 or 9,347,829, each of which is incorporated herein by reference. Analytes can be distributed to nanometer-scale posts, such as high aspect ratio posts which can optionally be dielectric pillars that extend through a metallic layer to improve detection of an analyte attached to the pillar. See, for example, U.S. Pat. Nos. 8,148,264, 9,410,887 or 9,987,609, each of which is incorporated herein by reference. Further examples of nanostructures that can be used to detect analytes are those that change state in response to the concentration of analytes such that the analytes can be quantitated as set forth in WO 2020/176793 A1, which is incorporated herein by reference.

An apparatus or method set forth herein need not be configured for optical detection. For example, an electronic detector can be used for detection of protons or charged labels (see, for example, US Pat. App. Pub. Nos. 2009/0026082 A1; 2009/0127589 A1; 2010/0137143 A1; or 2010/0282617 A1, each of which is incorporated herein by reference in its entirety). A field effect transistor (FET) can be used to detect analytes or other entities, for example, based on proximity of a field disrupting moiety to the FET. The field disrupting moiety can be due to an extrinsic label attached to an analyte or affinity agent, or the moiety can be intrinsic to the analyte or affinity agent being used. Surface plasmon resonance can be used to detect binding of analytes or affinity agents at or near a surface. Exemplary sensors and methods for attaching molecules to sensors are set forth in US Pat. App. Pub. Nos. 2017/0240962 A1; 2018/0051316 A1; 2018/0112265 A1; 2018/0155773 A1 or 2018/0305727 A1; or U.S. Pat. Nos. 9,164,053; 9,829,456; 10,036,064, each of which is incorporated herein by reference.

A composition, apparatus or method of the present disclosure can be used to characterize or identify at least about 0.0000001%, 0.000001%, 0.00001%, 0.0001%, 0.001%, 0.01%, 0.1%, 1%, 10%, 25%, 50%, 90%, 99%, 99.9%, 99.99%, 99.999%, 99.9999%, 99.99999%, 99.999999%, or more of all protein species in a proteome. Alternatively or additionally, a proteomic characterization method, in some embodiments, characterizes or no more than about 99.999999%, 99.99999%, 99.9999%, 99.999%, 99.99%, 99.9%, 99%, 90%, 50%, 25%, 10%, 1%, 0.1%, 0.01%, 0.001%, 0.0001%, 0.00001%, 0.000001%, 0.0000001%, or less of all protein species in a proteome.

In some configurations of the compositions, apparatus and methods set forth herein, one or more proteins can be present on a solid support, where the proteins can optionally be detected. For example, a protein can be attached to a solid support, the solid support can be contacted with a detection agent (e.g. affinity agent) in solution, the affinity agent can interact with the protein, thereby producing a detectable signal, and then the signal can be detected to determine the presence, absence, quantity, a characteristic or identity of the protein. In multiplexed versions of this approach, different proteins can be attached to different addresses in an array, and the detection steps can occur in parallel, such that proteins at each address are detected, quantified, characterized or identified. In another example, detection agents can be attached to a solid support, the support can be contacted with proteins in solution, the proteins can interact with the detection agents, thereby producing a detectable signal, and then the signal can be detected to determine the presence of the proteins. This approach can also be multiplexed by attaching different probes to different addresses of an array.

In multiplexed configurations, different proteins can be attached to different unique identifiers (e.g. addresses in an array), and the proteins can be manipulated and detected in parallel. For example, a fluid containing one or more different affinity agents can be delivered to an array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 1×10⁵ or more different native-length protein primary sequences. Alternatively or additionally, a proteome, proteome subfraction or other protein sample that is analyzed in a method set forth herein can have a complexity that is at most 1×10⁵, 1×10⁴, 1×10³, 100, 10, 5 or fewer different native-length protein primary sequences. The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, or loss of some proteins prior to analysis.

A particularly useful multiplex format uses an array of proteins and/or affinity agents. As used herein, the term “array” refers to a population of analytes (e.g. proteins) that are attached to unique identifiers such that the analytes can be distinguished from each other. As used herein, the term “unique identifier” refers to a solid support (e.g. particle or bead), spatial address in an array, tag, label (e.g. luminophore), or barcode (e.g. nucleic acid barcode) that is attached to an analyte and that is distinct from other identifiers, throughout one or more steps of a process. The process can be an analytical process such as a method for detecting, identifying, characterizing or quantifying an analyte. Attachment to a unique identifier can be covalent or non-covalent (e.g. ionic bond, hydrogen bond, van der Waals forces etc.). A unique identifier can be exogenous to the analyte, for example, being synthetically attached to the analyte. Alternatively, a unique identifier can be endogenous to the analyte, for example, being attached or associated with the analyte in the native milieu of the analyte. An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses. As used herein, the term “single-analyte array” refers to an array comprising one or more single-analytes. A single-analyte array, in some embodiments, comprises a plurality of addresses, in which each address contains a coupled single-analyte. A single-analyte array, in some embodiments, comprises a plurality of addresses, in which each address contains one and only one coupled single-analyte. A single-analyte array, in some embodiments, comprises a plurality of addresses, in which an address contains more than one coupled single-analyte.

As used herein, the term “address,” when used in reference to an array, means a location in an array where a particular analyte (e.g. protein) is present. An address can contain a single-analyte, or it can contain a population of several analytes of the same species (i.e. an ensemble of the analytes). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁸, 1×10¹⁰, 1×10¹², or more addresses. As used herein, the term “binding site,” when used in reference to an array, refers to a region of an address that is coupled to or configured to be coupled to an analyte. An address of an array, in some embodiments, comprises a binding site and a non-binding region. For example, an address of an array, in some embodiments, comprises a first region containing oligonucleotides that are complementary to an analyte surrounded by a second region containing a passivating layer, such as a monolayer of polyethylene glycol (PEG) molecules.

A protein can be attached to a unique identifier using any of a variety of means. The attachment can be covalent or non-covalent. Exemplary covalent attachments include chemical linkers such as those achieved using click chemistry or other linkages known in the art or described in U.S. Pat. No. 11,203,612, which is incorporated herein by reference. Non-covalent attachment can be mediated by receptor-ligand interactions (e.g. (strept)avidin-biotin, antibody-antigen, or complementary nucleic acid strands), for example, wherein the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in U.S. Pat. No. 11,203,612 and 11,505,796, each of which is incorporated herein by reference.

A solid support or a surface thereof, in some embodiments, is configured to display an analyte or a plurality of analytes. A solid support, in some embodiments, contains one or more patterned, formed, or prepared surfaces that contain at least one address for displaying an analyte. In some cases, a solid support contains one or more patterned, formed, or prepared surfaces that contain a plurality of addresses, with each address configured to display one or more analytes. Accordingly, an array as set forth herein, in some embodiments, comprises a plurality of analytes coupled to a solid support or a surface thereof. In some configurations, a solid support or a surface thereof is patterned or formed to produce an ordered or patterned array of addresses. The deposition of analytes on the ordered or patterned array of addresses, in some embodiments, is controlled by interactions between the solid support and the analytes such as, for example, electrostatic interactions, magnetic interactions, hydrophobic interactions, hydrophilic interactions, covalent interactions, or non-covalent interactions. Accordingly, the coupling of an analyte at each address of an array, in some embodiments, produces an ordered or patterned array of analytes whose average spacing between analytes is determined based upon the tolerance of the ordering or patterning of the solid support and the size of an analyte-binding region for each address. An ordered or patterned array of analytes, in some embodiments, is characterized as having a regular geometry, such as a rectangular, triangular, polygonal, or annular grid. In other configurations, a solid support or a surface thereof, in some embodiments, is non-patterned or non-ordered. The deposition of analytes on the non-ordered or non-patterned array of addresses, in some embodiments, is controlled by interactions between the solid support and the analytes, or inter-analyte interactions such as, for example, steric repulsion, electrostatic repulsion, electrostatic attraction, magnetic repulsion, magnetic attraction, covalent interactions, or non-covalent interactions.

A solid support or a surface thereof, in some embodiments, contains one or more structures or features. A structure or feature, in some embodiments, comprises an elevation, profile, shape, geometry, or configuration that deviates from an average elevation, profile, shape, geometry, or configuration of a solid support or surface thereof. A structure or feature, in some embodiments, is a raised structure or feature, such as a ridge, post, pillar, or pad, if the structure or feature extends above the average elevation of a surface of a solid support. A structure or feature, in some embodiments, is a depressed structure, such as a channel, well, pore, or hole, if the structure or feature extends below the average elevation of a surface of a solid support. A structure or feature, in some embodiments, is an intrinsic structure or feature of a substrate (i.e., arising due to the physical or chemical properties of the substrate, or a physical or chemical mechanism of formation), such as surface roughness structures, crystal structures, or porosity. A structure or feature, in some embodiments, is formed by a method of processing a solid support. In some configurations, a solid support or a surface is processed by a lithographic method to form one or more structures or features. A solid support or a surface thereof, in some embodiments, is formed by a suitable lithographic method, including, but not limited to photolithography, Dip-Pen nanolithography, nanoimprint lithography, nanosphere lithography, nanoball lithography, nanopillar arrays, nanowire lithography, immersion lithography, neutral particle lithography, plasmonic lithography, scanning probe lithography, thermochemical lithography, thermal scanning probe lithography, local oxidation nanolithography, molecular self-assembly, stencil lithography, laser interference lithography, soft lithography, magnetolithography, stereolithography, deep ultraviolet lithography, x-ray lithography, ion projection lithography, proton-beam lithography, or electron-beam lithography.

A solid support or surface, in some embodiments, comprises a plurality of structures or features. A plurality of structures or features, in some embodiments, comprises an ordered or patterned array of structures or features. A plurality of structures or features, in some embodiments, comprises a non-ordered, non-patterned, or random array of structures or features. A structure or feature, in some embodiments, has an average characteristic dimension (e.g., length, width, height, diameter, circumference, etc.) of at least about 1 nanometer (nm), 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1000 nm, or more than 1000 nm. Alternatively or additionally, a structure or feature, in some embodiments, has an average characteristic dimension of no more than about 1000 nm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm. An array of structures or features, in some embodiments, has an average pitch, in which the pitch is measured as the average separation between respective centerpoints of neighboring structures or features. An array, in some embodiments, has an average pitch of at least about 1 nm, 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1 micron (μm), 2 μm, 5 μm, 10 μm, 50 μm, 100 μm, or more than 100 μm. Alternatively or additionally, an array, in some embodiments, has an average pitch of no more than about 100 μm, 50 μm, 10 μm, 5 μm, 1 μm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm.

A solid support or a surface thereof, in some embodiments, includes a base substrate material and, optionally, one or more additional materials that are contacted or adhered with the substrate material. A solid support, in some embodiments, comprises one or more additional materials that are deposited, coated, or inlayed onto the substrate material. Additional materials, in some embodiments, are added to the substrate material to alter the properties of the substrate material. For example, materials, in some embodiments, are added to alter the surface chemistry (e.g., hydrophobicity, hydrophilicity, non-specific binding, electrostatic properties), alter the optical properties (e.g., reflective properties, refractive properties), alter the electrical or magnetic properties (e.g., dielectric materials, conducting materials, electrically-insulating materials), or alter the heat transfer characteristics of the substrate material. Additional materials contacted or adhered with a substrate material, in some embodiments, are ordered or patterned onto the substrate material to, for example, locate the additional material at addresses or locate the additional material at interstitial regions between addresses. Exemplary additional materials, in some embodiments, include metals (e.g., gold, silver, copper, etc.), metal oxides (e.g., titanium oxide, silicon dioxide, alumina, iron oxides, etc.), metal nitrides (e.g., silicon nitride, aluminum nitride, boron nitride, gallium nitride, etc.), metal carbides (e.g., tungsten carbide, titanium carbide, iron carbide, etc.), metal sulfides (e.g., iron sulfide, silver sulfide, etc.), and organic moieties (e.g., polyethylene glycol (PEG), dextrans, chemically-reactive functional groups, etc.).

A method of the present disclosure can include the step of coupling one or more analytes to a solid support or a surface thereof prior to performing a detection step set forth herein. The coupling of one or more analytes to a solid support surface, in some embodiments, includes covalent or non-covalent coupling of the one or more analytes to the solid support. Covalent coupling of an analyte to a solid support can include direct covalent coupling of an analyte to a solid support (e.g., formation of coordination bonds) or indirect covalent coupling between a reactive functional group of the analyte and a reactive functional group that is coupled to the solid support (e.g., a CLICK-type reaction). Non-covalent coupling can include the formation of any non-covalent interaction between an analyte and a solid support, including electrostatic or magnetic interactions, or non-covalent bonding interactions (e.g., ionic bonds, van der Waals interactions, hydrogen bonding, etc.). The skilled person will readily recognize that the particular analyte and the choice of solid support can affect the selection of a coupling chemistry for the compositions and methods set forth herein.

Accordingly, a coupling chemistry, in some embodiments, is selected based upon the criterium that it provides a sufficiently stable coupling of an analyte to a solid support for a time scale that meets or exceeds the time scale of a method as set forth herein. For example, a polypeptide identification method can require a coupling of the analyte to the solid support for a sufficient amount of time to permit a series of empirical measurements of the analyte to occur. An analyte, in some embodiments, is continuously coupled to a solid support for an observable length of time such as, for example, at least about 1 minute, 1 hour (hr), 3 hrs, 6 hrs, 12 hrs, 1 day, 1.5 days, 2 days, 3 days, 1 week (wk), 2 wks, 3 wks, 1 month, or more. The coupling of an analyte to a solid support can occur with a solution-phase chemistry that promotes the deposition of the analyte on the solid support. Coupling of an analyte to a solid support, in some embodiments, occurs under solution conditions that are optimized for any conceivable solution property, including solution composition, species concentrations, pH, ionic strength, solution temperature, etc. Solution composition can be varied by chemical species, such as buffer type, salts, acids, bases, and surfactants. In some configurations, species such as salts and surfactants, in some embodiments, are selected to facilitate the formation of interactions between an analyte and a solid support. Covalent coupling methods for coupling an analyte to a solid support, in some embodiments, include species such as catalyst, initiators, and promoters to facilitate particular reactive chemistries.

Coupling of an analyte to a solid support, in some embodiments, is facilitated by a mediating group. A mediating group, in some embodiments, modifies the properties of the analyte to facilitate the coupling. Useful mediating groups have been set forth herein (e.g., structured nucleic acid particles). In some configurations, a mediating group can be coupled to an analyte prior to coupling the analyte to a solid support. Accordingly, the mediating group, in some embodiments, is chosen to increase the strength, control, or specificity of the coupling of the analyte to the solid support. In other configurations, a mediating group can be coupled to a solid support prior to coupling an analyte to the solid support. Accordingly, the mediating group, in some embodiments, is chosen to provide a more favorable coupling chemistry than can be provided by the solid support alone.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

Notwithstanding the appended claims, the disclosure set forth herein is also defined by the following clauses:
1. A method of detecting one or more probe binding interactions on a single-analyte array, the method comprising:
   a. obtaining the single-analyte array comprising a first address that comprises a first probe complex and a second address that comprises a second probe complex, wherein the first probe complex comprises a first affinity reagent bound to a first single-analyte, wherein the first affinity reagent is configured to produce a first detectable signal, wherein the second probe complex comprises a second affinity reagent bound to a second single-analyte, wherein the second affinity reagent is configured to produce a second detectable signal, wherein the first detectable signal is optically distinguishable from the second detectable signal, and wherein the first address is spatially resolvable from the second address at single-analyte resolution;
   b. contacting the first address with a first excitation source, thereby producing the first detectable signal, wherein the first excitation source does not produce a detectable signal at the second address while contacting the first address;
   c. contacting the second address with a second excitation source, thereby producing the second detectable signal, wherein the second excitation source does not produce a detectable signal at the first address while contacting the first address; and
   d. detecting the first signal and the second signal on a sensor, wherein the sensor comprises a first channel that is configured to detect the first signal and a second channel that is configured to detect the second signal.
2. A method of detecting one or more probe binding interactions on a single-analyte array, the method comprising:
   a. obtaining the single-analyte array comprising a first address and a second address, wherein the first address comprises a first signal source that emits a first signal and the second address comprises a second signal source that emits a second signal, wherein the first address is spatially resolvable from the second address; and
   b. detecting the first signal and the second signal on an optical system comprising a sensor, wherein the sensor comprises a first channel that detects the first signal and a second channel that detects the second signal, wherein the first channel is spatially separated and coplanar with the second channel.
3. The method of clause 2, wherein the single-analyte array comprises an ordered array.
4. The method of clause 2, wherein the ordered array comprises a patterned array.
5. The method of clause 4, wherein the patterned array is formed by a lithographic method.
6. The method of clause 4 or 5, wherein the patterned array comprises a plurality of addresses arranged in a repeating pattern, wherein the plurality of addresses comprises the first address and the second address.
7. The method of clause 6, wherein the plurality of addresses is arranged in a rectangular pattern, a triangular pattern, a hexagonal pattern, or a radial pattern.
8. The method of clause 6 or 7, wherein the single-analyte array is coupled to a plurality of analytes of interest, and wherein an address of the plurality of addresses is coupled to a single-analyte of interest in the plurality of analytes of interest.
9. The method of any one of clauses 6-8, wherein the average pitch for the plurality of addresses has a coefficient of variation of no more than 10%.
10. The method of clause 9, wherein the average pitch for the plurality of addresses has a coefficient of variation of no more than 1%.
11. The method of clause 2, wherein the single-analyte array comprises an unpatterned array having a non-repeating pattern of addresses.
12. The method of clause 11, wherein the unpatterned array comprises the first address and the second address.
13. The method of clause 12, wherein the average pitch for the plurality of addresses has a coefficient of variation of at least 1%.
14. The method of clause 13, wherein the average pitch for the plurality of addresses has a coefficient of variation of at least 10%.
15. The method of clause 14, wherein the average pitch for the plurality of addresses has a coefficient of variation of at least 50%.
16. The method of any one of clauses 2-15, wherein a single-analyte of the single-analyte array comprises a cell, a polypeptide, a nucleic acid, a metabolite, or a combination thereof.
17. The method of any one of clauses 2-16, wherein the single-analyte array comprises a solid support.
18. The method of clause 17, wherein the single-analyte array is disposed on a surface of the solid support.
19. The method of clause 18, wherein the surface of the solid support is substantially planar.
20. The method of clause 19, wherein the surface of the solid support comprises a plurality of structures, wherein each structure comprises a substantially planar face, and wherein each face of the plurality of structures is substantially coplanar with each other face of the plurality of structures.
21. The method of any one of clauses 17-20, wherein the solid support comprises a metal, metal oxide, semiconductor, glass, polymer, composite material, or a combination thereof.
22. The method of clause 21, wherein the solid support further comprises an organic layer or coating.
23. The method of clause 22, wherein the organic layer or coating comprises a passivating moiety.
24. The method of clause 23, wherein the passivating moiety inhibits non-specific binding of a molecule to the solid support.
25. The method of clause 23 or 24, wherein the passivating moiety comprises a hydrophobic moiety, a hydrophilic moiety, a polar moiety, or a non-polar moiety.
26. The method of any one of clauses 23-25, wherein the passivating moiety comprises a linear polyethylene glycol (PEG) moiety, a branched PEG moiety, a linear alkane moiety, a branched alkane moiety, a linear fluorinated hydrocarbon, a branched fluorinated hydrocarbon, or a combination thereof.
27. The method of clause 22, wherein the organic layer or coating comprises a coupling moiety.
28. The method of clause 27, wherein the coupling moiety couples a single-analyte to the solid support.
29. The method of clause 28, wherein the coupling moiety couples the single-analyte to the solid support by a covalent interaction.
30. The method of clause 29, wherein the coupling moiety comprises a Click-type reactive moiety.
31. The method of clause 29, wherein the coupling moiety couples the single-analyte to the solid support by a non-covalent interaction.
32. The method of clause 31, wherein the coupling moiety comprises an oligonucleotide, an electrically-charged moiety, a magnetic moiety, or a member of a receptor-ligand binding pair.
33. The method of any one of clauses 2-32, wherein the single-analyte array is disposed within a fluidic cartridge.
34. The method of clause 33, wherein the fluidic cartridge comprises an internal volume, wherein the single-analyte array is disposed within the internal volume.
35. The method of clause 33 or 34, wherein the fluidic cartridge comprises a material with an index of refraction of at least 1.0.
36. The method of clause 35, wherein the fluidic cartridge comprises a material with an index of refraction of at least 3.0.
37. The method of any one of clauses 2-36, wherein the first signal source or the second signal source comprises a fluorophore or a luminophore.
38. The method of clause 37, wherein the first signal source comprises a first fluorophore and the second signal source comprises a second fluorophore.
39. The method of clause 38, wherein the first fluorophore comprises a same chemical structure as the second fluorophore.
40. The method of clause 38 or 39, wherein the first fluorophore comprises a same peak absorption wavelength as the second fluorophore.
41. The method of any one of clauses 38-40, wherein the first fluorophore comprises a same peak emission wavelength as the second fluorophore.
42. The method of clause 38, wherein the first fluorophore comprises a different chemical structure than the second fluorophore.
43. The method of clause 41 or 42, wherein the first fluorophore comprises a different peak absorption wavelength than the second fluorophore.
44. The method of any one of clauses 41-43, wherein the first fluorophore comprises a different peak emission wavelength than the second fluorophore.
45. The method of any one of clauses 37-44, wherein the fluorophore is coupled to a single-analyte.
46. The method of clause 45, wherein the fluorophore is coupled to a linking moiety, wherein the linking moiety couples the single-analyte to the single-analyte array.
47. The method of any one of clauses 37-44, wherein the fluorophore is coupled to an affinity agent.
48. The method of clause 47, wherein the affinity agent is bound to a single-analyte.
49. The method of clause 37, wherein the first signal source or the second signal source comprises a probe complex, wherein the probe complex comprises an affinity agent bound to a single-analyte.
50. The method of clause 49, wherein the probe complex further comprises a linking moiety, wherein the linking moiety couples the single-analyte to the single-analyte array.
51. The method of any one of clauses 2-50, wherein the first address is physically separated from the second address by a separation distance, wherein the separation distance is at least a distance for distinguishing the first address from the second address at single-analyte resolution.
52. The method of clause 51, wherein the distance for distinguishing the first address from the second address at single-analyte resolution comprises a distance between a peak signal intensity of the first address and a peak signal intensity of the second address.
53. The method of clause 52, wherein the peak signal intensity of the first address and the peak signal intensity of the second address each have a magnitude that is at least twice the magnitude of a minimum signal intensity between the first address and the second address.
54. The method of clause 51, wherein the separation distance is at least 100 nanometers (nm).
55. The method of clause 54, wherein the separation distance is at least 1 μm.
56. The method of clause 55, wherein the separation distance is at least 10 μm.
57. The method of any one of clauses 51-56, wherein the first address and the second address are non-adjacent addresses of the single-analyte array.
58. The method of any one of clauses 51-56, wherein the first address and the second address are adjacent addresses of the single-analyte array.
59. The method of any one of clauses 2-58, wherein the sensor comprises a solid support comprising the first channel and the second channel.
60. The method of clause 59, wherein the first channel or the second channel comprises a pixel array.
61. The method of clause 60, wherein the pixel array comprises a charge-coupled device (CCD) pixel array or a complementary metal-oxide semiconductor (CMOS) pixel array.
62. The method of clause 60 or 61, wherein the pixel array comprises a rectangular pixel array,
wherein a total pixel quantity of the pixel array is determined as a product of a lengthwise number of pixels and a widthwise number of pixel.
63. The method of clause 62, wherein the rectangular pixel array has an aspect ratio of at least 5:1, wherein the aspect ratio is calculated as the ratio of the lengthwise number of pixels to the widthwise number of pixels.
64. The method of clause 63, wherein the pixel array has an aspect ratio of at least 10:1.
65. The method of any one of clauses 60-64, wherein the first channel comprise a first rectangular pixel array and the second channel comprise a second rectangular pixel array, wherein the first rectangular pixel array is oriented on the solid support parallel to the second rectangular pixel array.
66. The method of clause 65, wherein the first rectangular pixel array is spatially separated from the second rectangular pixel array by a channel separation distance.
67. The method of clause 65, wherein the channel separation distance is related to the separation distance of the first address and the second address by a magnification of the optical system.
68. The method of clause 67, wherein the magnification of the optical system is at least 10×.
69. The method of clause 68, wherein the magnification of the optical system is at least 40×.
70. The method of clause 69, wherein the magnification of the optical system is at least 100×.
71. The method of any one of clauses 59-70, wherein the sensor further comprises a filter adjacent to the first channel.
72. The method of clause 71, wherein the filter excludes light transmitted by the second signal source.
73. The method of clause 71 or 72, wherein the filter is disposed on an exposed surface of the first channel.
74. The method of clause 71 or 72, wherein the filter does not contact an exposed surface of the first channel.
75. The method of any one of clauses 2-74, wherein the sensor comprises a first solid support comprising the first channel and a second solid support comprising the second channel.
76. The method of any one of clauses 2-75, further comprising contacting the first signal source with a first excitant.
77. The method of any one of clauses 2-76, further comprising contacting the second signal source with a second excitant.
78. The method of clause 77, wherein the first excitant or the second excitant comprises an electromagnetic excitant, a chemical excitant, a thermal excitant, or a combination thereof.
79. The method of clause 78, wherein the electromagnetic excitant comprises a photon.
80. The method of any one of clauses 2-79, further comprising:
   i) forming a spatially-separated electromagnetic beam, wherein the spatially separated electromagnetic beam comprising a first excitant beam and a second excitant beam;
   ii) contacting the spatially-separated electromagnetic beam with the single-analyte array, wherein the first excitant beam contacts the first signal source, and wherein the second excitant beam contacts the second signal source.
81. The method of clause 80, wherein forming the spatially-separated electromagnetic beam comprises passing an electromagnetic beam through a beam-splitting optical device.
82. The method of clause 81, wherein the first excitant beam comprises a first wavelength of light, wherein the second beam comprises a second wavelength of light, and wherein the first wavelength of light differs from the second wavelength of light.
83. The method of clause 82, wherein the first wavelength of light differs from the second wavelength of light by at least about 50 nm.
84. The method of clause 81, wherein the first excitant beam and the second excitant beam comprise the same wavelength of light.
85. The method of any one of clauses 80-84, wherein the first excitant beam or the second excitant beam forms a line of light when contacted with the single-analyte array.
86. The method of clause 85, wherein the first excitant beam forms a first line of light and the second excitant beam forms a second line of light, wherein the first line of light is parallel to the second line of light on the single-analyte array.
87. The method of clause 86, wherein the first channel of the sensor is oriented parallel to the first line of light, and the second channel of the sensor is oriented parallel to the second line of light.
88. The method of any one of clauses 80-84, wherein the first excitant beam or the second excitant beam forms a point of light on the single-analyte array.
89. The method of any one of clauses 2-88, wherein detecting the first signal and the second signal on the optical system comprising the sensor comprises one or more steps of:
   i) passing the first signal and the second signal through an objective of the optical system;
   ii) passing the first signal and the second signal through a focusing device of the optical system; and
   iii) contacting the first signal with the first channel of the sensor, and contacting the second signal with the second channel of the sensor.
90. The method of clause 89, wherein passing the first signal and the second signal through the focusing device further comprises simultaneously adjusting focus of the first signal and the second signal on the sensor.
91. The method of clause 89 or 90, wherein the objective lens comprises a numerical aperture of at least 0.6.
92. The method of clause 91, wherein the objective lens comprises a numerical aperture of at least 0.8.
93. The method of any one of clauses 89-92, wherein the objective lens comprises an air-immersion objective lens.
94. The method of any one of clauses 89-92, wherein the objective lens comprises a water-immersion objective lens or an oil-immersion objective lens.
95. The method of any one of clauses 2-94, wherein the optical system comprises a confocal microscope system.
96. The method of clause 95, wherein the confocal microscope system comprises a linescan confocal microscope system.
97. A method of detecting one or more probe binding interactions on a single-analyte array, the method comprising:
a. translating the relative positions of the single-analyte array and an optical system in time-delay integration mode, wherein the single-analyte array comprises a first address and a second address, wherein the first address comprises a first optical signal source that emits a first optical signal and the second address comprises a second optical signal source that emits a second optical signal, wherein the first address is physically separated from the second address by a distance that is resolvable at single-analyte resolution; and
b. detecting the first signal and the second signal on a sensor, wherein the first optical signal is detected on a first channel of the sensor and the second optical signal is detected on a second channel of the sensor, and wherein focus of the first optical signal and the second optical signal on the sensor are not separately adjusted over the duration of a time-delay integration scan of the single-analyte array.
98. The method of clause 97, wherein the optical system is non-isothermal.
99. The method of clause 98, wherein the non-isothermal optical system comprises one or more internal heat sources.
100. The method of clause 98, wherein the non-isothermal optical system comprises an external heat source.
101. The method of any one of clauses 98-100, wherein the optical system is spatially non-isothermal.
102. The method of any one of clauses 98-101, wherein the optical system is temporally non-isothermal.
103. The method of any one of clauses 98-102, wherein the optical system operates in a synchronous time delay integration mode.
104. The method of any one of clauses 98-103, wherein the optical system operates in an asynchronous time delay integration mode.
105. The method of any one of clauses 98-104, further comprising one or more steps of:
   i) passing the first signal and the second signal through a focusing device;
   ii) detecting the first signal and the second signal on the sensor;
   iii) based upon the detected first signal and second signal, identifying a focus correction for the focusing device;
   iv) after applying the focus correction to the focusing device, detecting the first signal and the second signal on the sensor.
106. A multiplex optical system, comprising:
   a. two or more light sources, wherein a first light source is configured to transmit light of a first wavelength, and wherein a second light source is configured to transmit light of a second wavelength, wherein the first wavelength differs from the second wavelength;
   b. a stage that is configured to accommodate a fluidic cartridge;
   c. an objective lens; and
   d. an optical sensor, wherein the optical sensor comprises a first channel and a second channel, wherein the first channel and the second channel are spatially separated on the sensor, and wherein the first channel is configured to detect the light of the first wavelength, and wherein the second channel is configured to detect the light of the second wavelength.
107. The multiplex optical system of clause 106, wherein the first light source or the second light source comprises a light source selected from the group consisting of a laser, a lamp, a bulb, a filament, a light-emitting diode (LED), and a combination thereof.
108. The multiplex optical system of clause 107, wherein the light source is configured to transmit light from the ultraviolet, visible, or infrared region of the electromagnetic spectrum.
109. The multiplex optical system of clause 107 or 108, wherein the light source is configured to produce light over a range of wavelengths, wherein the range of wavelengths includes the first wavelength or the second wavelength.
110. The multiplex optical system of clause 107 or 108, wherein the light source is configured to produce light of substantially a single wavelength, wherein the single wavelength is the first wavelength or the second wavelength.
111. The multiplex optical system of clause 110, wherein the light source is configured to produce at least 90% light of the single wavelength.
112. The multiplex optical system of any one of clauses 107-111, further comprising a light pipe, wherein the light pipe is configured to transmit light from the first light source and light from the second light source to a beam-shaping optical device.
113. The multiplex optical system of clause 112, wherein the light pipe comprises a fiber optic light pipe.
114. The multiplex optical system of clause 113, wherein the fiber optic light pipe comprises two or more optical cores, wherein a first optical core is configured to transmit light from the first light source, and wherein a second optical core is configured to transmit light from the second light source.
115. The multiplex optical system of any one of clauses 112-114, wherein the beam-shaping optical device comprises a beam splitter.
116. The multiplex optical system of clause 115, wherein the beam splitter is configured to spatially separate light from the first light source from light from the second light source.
117. The multiplex optical system of clause 116, wherein light from the first light source is separated from light from the second light source by a distance that is at least the distance that separates the first channel from the second channel on the sensor.
118. The multiplex optical system of any one of clauses 106-117, further comprising a translation system, wherein the translation system is configured to adjust the relative position of the fluidic cartridge and the objective lens.
119. The multiplex optical system of clause 118, wherein the translation system alters the position of the stage.
120. The multiplex optical system of clause 118, wherein the translation system alters the position of the sensor or the objective lens.
121. The multiplex optical system of any one of clauses 118-120, wherein the translation system comprises an x-y motion controller, an x-y-z motion controller, a rotational motion controller, or a combination thereof.
122. The multiplex optical system of any one of clauses 118-121, wherein the translation system is configured to produce a motion of the fluidic cartridge that is substantially orthogonal to the objective lens.
123. The multiplex optical system of any one of clauses 118-122, wherein the multiplex optical system comprises a magnification of at least 10×.
124. The multiplex optical system of clause 123, wherein the multiplex optical system comprises a magnification of at least 40×.
125. The multiplex optical system of clause 124, wherein the multiplex optical system comprises a magnification of at least 100×.
126. The multiplex optical system of any one of clauses 123-125, wherein the multiplex optical system comprises a numerical aperture of at least 0.5.
127. The multiplex optical system of clause 126, wherein the multiplex optical system comprises a numerical aperture of at least 0.6.
128. The multiplex optical system of clause 127, wherein the multiplex optical system comprises a numerical aperture of at least 0.8.
129. The multiplex optical system of any one of clauses 106-128, further comprising a focusing device.
130. The multiplex optical system of clause 129, wherein the focusing device is configured to focus one or more light sources on the sensor.
131. The multiplex optical system of clause 129 or 130, wherein the system further comprises a processor that is configured to receive optical data from the sensor and send a control instruction to the focusing device based upon the received optical data.
132. The multiplex optical system of any one of clauses 106-131, further comprising an optomechanical structure.
133. The multiplex optical system of clause 132, wherein the optomechanical structure is configured to couple the sensor to one or more additional optical components.
134. The multiplex optical system of clause 133, wherein the one or more additional optical components comprise a beam-splitting device, a beam-shaping device, the objective lens, the focusing device, or a combination thereof.

What is claimed is:

1. A method, comprising:
a) providing a solid support comprising an address, wherein the address is resolvable at single-analyte resolution, wherein the address comprises an analyte, wherein the analyte is coupled to the address by a linking moiety, wherein the linking moiety comprises a first optically detectable label, wherein a probe is bound to the analyte, wherein the probe comprises a second optically detectable label, wherein the first optically detectable label produces a first optical signal of a first wavelength, wherein the second optically detectable label produces a second optical signal of a second wavelength, and wherein the first wavelength differs from the second wavelength;
b) detecting on a first channel of a sensor a presence of the first detectable signal at the address, and detecting on a second channel of the sensor a presence of the second detectable signal;
c) removing the probe from the analyte; and
d) after removing the probe from the analyte, detecting on the first channel of the sensor the presence of the first detectable signal, and detecting on the second channel an absence of the second detectable signal.

2. The method of claim 1, wherein detecting on the sensor occurs in time delay and integration (TDI) mode.

3. The method of claim 1, wherein the first channel comprises a first array of light-sensing elements, and the second channel comprises second array of light-sensing elements.

4. The method of claim 3, wherein the first channel and the second channel are coplanar and spatially separated on a solid support.

5. The method of claim 1, wherein the linking moiety comprises a nucleic acid.

6. The method of claim 5, wherein the nucleic acid comprises a nucleic acid nanoparticle.

7. The method of claim 1, wherein the probe further comprises an affinity agent.

8. The method of claim 7, wherein the probe further comprises a plurality of affinity agents.

9. The method of claim 8, wherein the plurality of affinity agents is coupled to the second detectable label by a retaining moiety.

10. The method of claim 9, wherein the retaining moiety comprises a nucleic acid nanoparticle.

11. The method of claim 1, wherein the probe is non-covalently bound to the analyte.

12. The method of claim 11, wherein removing the probe comprises dissociating the probe from the analyte.

13. The method of claim 1, wherein the probe is covalently bound to the analyte.

14. The method of claim 13, wherein removing the probe comprises chemically or enzymatically separating the probe from the analyte.

15. The method of claim 14, wherein chemically separating the probe from the analyte comprises performing an Edman-type degradation reaction.

16. The method of claim 1, further comprising: e) contacting a second probe to the array containing the analyte, wherein the second probe comprises the second optically detectable label.

17. The method of claim 16, further comprising: f) detecting on the second channel of the sensor a presence or an absence of the second detectable signal at the address.

18. The method of claim 17, wherein a temperature change of at least 1 degree Celsius occurs between step b) and step f).

19. The method of claim 18, further comprising, before step b), adjusting a focus of an optical detection system.

20. The method of claim 19, wherein the focus of the optical detection system is not adjusted between step b) and step f).
